SEQUENCE LISTING (2 sequences)

SEQ ID NO: 1 - protein, 452 aa, synthetic construct
  DOMAIN 1..452: note = Amino Acid sequence of the Heavy Chain of Enoblituzumab
EVQLVESGGGLVQPGGSLRLSCAASGFTFSSFGMHWVRQAPGKGLEWVAYISSDSSAIYYADTVKGRFTISRDNAKNSLYLQMNSLRDEDTAVYYCGRGRENIYYGSRLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELVGGPSVFLLPPKPKDTLMISRTPEVTCVVVDVSHEDPEVKFNWYVDGVEVHNAKTKPPEEQYNSTLRVVSVLTVLHQDWLNGKEYKCKVSNKALPAPIEKTISKAKGQPREPQVYTLPPSREEMTKNQVSLTCLVKGFYPSDIAVEWESNGQPENNYKTTPLVLDSDGSFFLYSKLTVDKSRWQQGNVFSCSVMHEALHNHYTQKSLSLSPGK

SEQ ID NO: 2 - protein, 214 aa, synthetic construct
  DOMAIN 1..214: note = Amino Acid Sequence of the Light Chain of Enoblituzumab
DIQLTQSPSFLSASVGDRVTITCKASQNVDTNVAWYQQKPGKAPKALIYSASYRYSGVPSRFSGSGSGTDFTLTISSLQPEDFATYYCQQYNNYPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2024/0336687 A1
(43) Pub. Date: Oct. 10, 2024

(54) PHARMACEUTICAL COMPOSITIONS OF A B7-H3 ANTIBODY AND USE OF THE SAME

(71) Applicant: MACROGENICS, INC., Rockville, MD (US)

(72) Inventors: Yan ZHOU, Rockville, MD (US); Krishnan SAMPATHKUMAR, Rockville, MD (US); Stephen James BURKE, Rockville, MD (US); Xiaoyan WANG, Rockville, MD (US)

(21) Appl. No.: 18/577,945

(22) PCT Filed: Jul. 6, 2022
(86) PCT No.: PCT/US2022/073462

§ 371 Date: Jan. 9, 2024

Related U.S. Application Data
(60) Provisional application No. 63/222,750, filed on Jul. 16, 2021.

(51) Int. Cl. C07K 16/28; A61K 47/12; A61K 47/26

(52) U.S. Cl. CPC C07K 16/2827; A61K 47/12; A61K 47/26; C07K 2317/24

(57) ABSTRACT

The present invention provides pharmaceutical compositions, for storage and administration, comprising an anti-human B7-H3 (“hB7-H3”) antibody (“enoblituzumab”) and buffering agents. The invention provides containers and kits comprising such pharmaceutical compositions. The invention further provides the use of such pharmaceutical compositions, containers, and kits containing enoblituzumab for the treatment of a cancer, and in certain aspects, treatment of a cancer expressing B7-H3.

### CROSS-REFERENCE TO RELATED APPLICATIONS

This application claims priority to U.S. Patent Application Ser. No. 63/222,750 (filed on Jul. 16, 2021; pending), which application is herein incorporated by reference in its entirety.

### REFERENCE TO SEQUENCE LISTING

This application includes one or more Sequence Listings pursuant to 37 C.F.R. 1.821 et seq., which are disclosed in computer-readable media (file name: 1301_0169PCT_TW.xml, created on Jun. 2, 2022, and having a size of 4,307 bytes), which file is herein incorporated by reference in its entirety.

### FIELD

The present invention provides pharmaceutical compositions, for storage and administration, comprising an anti-human B7-H3 (“hB7-H3”) antibody (“enoblituzumab”) and buffering agents. The invention further provides containers and kits comprising such pharmaceutical compositions. The invention further provides the use of such pharmaceutical compositions, containers, and kits containing enoblituzumab for the treatment of a cancer, and in certain aspects, treatment of a cancer expressing B7-H3.

### BACKGROUND

B7-H3 (also known as “CD276”) is expressed on antigen-presenting cells and binds to an unidentified receptor(s) to mediate co-inhibition of T cells. In addition, B7-H3, through interactions with unknown receptor(s) is an inhibitor for NK-cells and osteoblastic cells (Hofmeyer, K. et al. (2008) “The Contrasting Role Of B7-H3,” Proc. Natl. Acad. Sci. (U.S.A.) 105(30):10277-10278). B7-H3 is expressed on a variety of cancer cells (e.g., neuroblastoma, gastric, ovarian, non-small cell lung cancers, etc., see, e.g., Modak, S., et al. (2001) “Monoclonal antibody 8H9 targets a novel cell surface antigen expressed by a wide spectrum of human solid tumors,” Cancer Res 61:4048-54). The role of B7-H3 in inhibiting the immune system and the increased expression of B7-H3 on human tumors suggests that this molecule might serve as a therapeutic target for the treatment of cancer. The use of anti-hB7-H3 antibodies and other molecules that modulate B7-H3 expression to treat tumors and/or up-modulate an immune response has been reported (see, e.g., U.S. Pat. No. 8,802,091).

### SUMMARY

In one aspect, the present invention provides pharmaceutical compositions for patients whose tumors express B7-H3, including those whose tumors express low levels of B7-H3 or who have failed on other therapies. The present invention further provides pharmaceutical compositions comprising enoblituzumab. The invention further provides the use of such pharmaceutical compositions and pharmaceutical kits that that contain such pharmaceutical compositions for the treatment of cancer, and in certain aspects, treatment of a cancer expressing B7-H3, for example with a therapeutically effective amount or prophylactically effective amount of enoblituzumab.

In one embodiment, the invention provides pharmaceutical compositions that maintain the stability of enoblituzumab across a broad concentration range, about 5 mg/mL to about 200 mg/mL, including lower concentrations (e.g., about 5 mg/mL to about 60 mg/mL) and higher concentrations (e.g., about 60 mg/mL to about 200 mg/mL). The invention further provides pharmaceutical compositions comprising enoblituzumab, acetate, sucrose, polysorbate 80 (“PS80”), and water.

The invention provides an embodiment of such pharmaceutical compositions, wherein the acetate has a concentration of about 5 mM to about 30 mM. The invention further provides an embodiment of such pharmaceutical compositions, wherein the acetate comprises sodium acetate. The invention provides an embodiment of such pharmaceutical compositions, wherein the acetate comprises glacial acetic acid and sodium acetate.

The invention provides an embodiment of such pharmaceutical compositions, wherein the composition comprises:
a) about 5 mM to about 30 mM acetate, about 50 mg/mL to about 130 mg/mL of sucrose, about 0.05 mg/mL to about 0.6 mg/mL of PS80, and water, wherein the composition has a pH of about 4.0 to about 6.0; or
b) about 8 mM to about 24 mM acetate, about 72 mg/mL to about 108 mg/mL of sucrose, about 0.05 mg/mL to about 0.6 mg/mL of PS80, and water, wherein the composition has a pH of about 4.4 to about 5.6; or
c) about 16 mM to about 24 mM acetate, about 72 mg/mL to about 108 mg/mL of sucrose, about 0.05 mg/mL to about 0.2 mg/mL of PS80, and water, wherein the composition has a pH of about 4.3 to about 5.3; or
d) about 8 mM to about 12 mM acetate, about 72 mg/mL to about 108 mg/mL of sucrose, about 0.05 mg/mL to about 0.2 mg/mL of PS80, and water, wherein the composition has a pH of about 4.5 to about 5.5; or
e) about 10 mM acetate, about 90 mg/mL of sucrose, about 0.1 mg/mL of PS80, and water, wherein the composition has a pH of about 4.6 to about 5.5; or
f) about 20 mM acetate, about 90 mg/mL of sucrose, about 0.1 mg/mL of PS80, and water, wherein the composition has a pH of about 4.4 to about 5.2.

The invention provides an embodiment of such pharmaceutical compositions, wherein the acetate comprises glacial acetic acid at a concentration of about 0.1 mg/mL to about 0.65 mg/mL and sodium acetate trihydrate at a concentration of about 0.6 mg/mL to about 1.8 mg/mL.

The invention provides an embodiment of such pharmaceutical compositions, wherein the enoblituzumab has a concentration of about 5 mg/mL to about 60 mg/mL. The invention further provides an embodiment of such pharmaceutical compositions, wherein the enoblituzumab has a concentration of about 20 mg/mL to about 30 mg/mL. The invention further provides an embodiment of such pharmaceutical compositions, wherein the enoblituzumab has a concentration of about 25 mg/mL.

The invention provides an embodiment of such pharmaceutical compositions, wherein the acetate comprises glacial acetic acid at a concentration of about 0.1 mg/mL to about 0.35 mg/mL and sodium acetate trihydrate at a concentration of about 0.60 mg/mL to about 1.2 mg/mL. The invention further provides an embodiment of such pharmaceutical compositions, wherein the acetate comprises glacial acetic acid at a concentration of about 0.18 mg/mL and sodium acetate trihydrate at a concentration of about 0.95 mg/mL. The invention further provides an embodiment of such pharmaceutical compositions, wherein the acetate comprises glacial acetic acid at a concentration of about 0.27 mg/mL and sodium acetate trihydrate at a concentration of about 0.74 mg/mL.

The invention provides an embodiment of such pharmaceutical compositions, wherein the enoblituzumab has a concentration of about 90 mg/mL to about 200 mg/mL. The invention further provides an embodiment of such pharmaceutical compositions, wherein the enoblituzumab has a concentration of about 120 mg/mL.

The invention provides an embodiment of such pharmaceutical compositions, wherein the acetate comprises glacial acetic acid at a concentration of about 0.4 mg/mL to about 0.65 mg/mL and sodium acetate trihydrate at a concentration of about 1.2 mg/mL to about 1.8 mg/mL. The invention further provides an embodiment of such pharmaceutical compositions, wherein the acetate comprises glacial acetic acid at a concentration of about 0.52 mg/mL and sodium acetate trihydrate at a concentration of about 1.5 mg/mL.

The invention provides an embodiment of such pharmaceutical compositions, wherein the concentration of sucrose is present at a concentration of about 50 mg/mL to about 130 mg/mL. The invention further provides an embodiment of such pharmaceutical compositions, wherein the concentration of sucrose is present at a concentration of about 72 mg/mL to about 108 mg/mL. The invention further provides an embodiment of such pharmaceutical compositions, wherein the concentration of sucrose is about 90 mg/mL.

The invention provides an embodiment of such pharmaceutical compositions, wherein the PS80 is present at a concentration of about 0.05 mg/mL to about 0.6 mg/mL. The invention further provides an embodiment of such pharmaceutical compositions, wherein the PS80 is present at a concentration of about 0.08 mg/mL to about 0.15 mg/mL. The invention further provides an embodiment of such pharmaceutical compositions, wherein the concentration of PS80 is about 0.1 mg/mL.

The invention provides an embodiment of such pharmaceutical compositions, wherein the composition has a pH of about 4.6 to about 5.5. The invention further provides an embodiment of such pharmaceutical compositions, wherein the composition has a pH of about 4.4 to about 5.2.

The invention provides an embodiment of such pharmaceutical compositions, wherein the composition comprises about 25 mg/mL of enoblituzumab, about 0.18 mg/mL of glacial acetic acid, about 0.95 mg/mL of sodium acetate trihydrate, about 90 mg/mL of sucrose, about 0.1 mg/mL of PS80, and water, wherein the composition has a pH of about 4.7 to about 5.5.

The invention provides an embodiment of such pharmaceutical compositions, wherein the composition comprises about 25 mg/mL of enoblituzumab, about 0.27 mg/mL of glacial acetic acid, about 0.74 mg/mL of sodium acetate trihydrate, about 90 mg/mL of sucrose, about 0.1 mg/mL of PS80, and water, wherein the composition has a pH of about 4.6 to about 5.4.

The invention provides an embodiment of such pharmaceutical compositions, wherein the composition comprises about 120 mg/mL of enoblituzumab, about 0.52 mg/mL of glacial acetic acid, about 1.5 mg/mL of sodium acetate trihydrate, about 90 mg/mL of sucrose, about 0.1 mg/mL of PS80, and water, wherein the composition has a pH of about 4.4 to about 5.2.

The invention provides an embodiment of such pharmaceutical compositions, wherein the composition does not comprise an antioxidant.

The invention provides an embodiment of such pharmaceutical compositions, wherein the composition has a shelf-life of at least about 18 months at about 2° C. to about 8° C.

The invention further provides an embodiment of such pharmaceutical compositions, wherein the composition has a shelf-life of at least about 24 months at about 2° C. to about 8° C. The invention further provides an embodiment of such pharmaceutical compositions, wherein the composition has a shelf-life of at least about 36 months at about 2° C. to about 8° C. The invention further provides an embodiment of such pharmaceutical compositions, wherein the composition has a shelf-life of at least about 48 months at about 2° C. to about 8° C.

The invention provides an embodiment of such pharmaceutical compositions, wherein the composition has an osmolality of about 200 to about 400 mOsm/kg H₂O. The invention further provides an embodiment of such pharmaceutical compositions, wherein the composition has an osmolality of about 260 to about 360 mOsm/kg H₂O.

The invention provides an embodiment of such pharmaceutical compositions, wherein the composition maintains monomeric purity of the enoblituzumab for at least about 3 months at about 25° C. The invention further provides an embodiment of such pharmaceutical compositions, wherein the composition maintains monomeric purity of the enoblituzumab for at least about 18 months at about 2° C. to about 8° C.

The invention provides an embodiment of such pharmaceutical compositions, wherein the composition maintains the heterogeneity profile of the enoblituzumab for about for at least about 3 months at 25° C. The invention further provides an embodiment of such pharmaceutical compositions, wherein the composition maintains the heterogeneity profile of the enoblituzumab for about for at least about 18 months at about 2° C. to about 8° C.

The invention provides an embodiment of such pharmaceutical compositions, wherein the water is sterile, nonpyrogenic, distilled water.

The invention provides an embodiment of such pharmaceutical compositions, wherein the composition is sterile.

The invention provides a container comprising any of the pharmaceutical compositions disclosed herein. The invention further provides an embodiment of such container, wherein such container comprises about 5 mL volume, about 10 mL volume, about 15 mL volume, about 17 mL volume, or about 20 mL volume of such pharmaceutical compositions.

The invention provides an embodiment of such containers, wherein the container comprises about 10 mL volume of the pharmaceutical composition, wherein volume comprises: (a) about 250 mg enoblituzumab; (b) about 10 mM sodium acetate; (c) about 900 mg sucrose; (d) about 1 mg PS80; and (f) water; and wherein the composition has a pH of about 4.6 to about 5.5.

The invention provides an embodiment of such containers, wherein the container comprises about 10 mL volume of the pharmaceutical composition, wherein the volume comprises: (a) about 250 mg enoblituzumab; (b) about 1.8 mg glacial acetic acid; (c) about 9.5 mg sodium acetate trihydrate; (d) about 900 mg sucrose; (e) about 1 mg PS80; and (f) water; and wherein the composition has a pH of about 4.7 to about 5.5.

The invention provides an embodiment of such containers, wherein the container comprises about 10 mL volume of the pharmaceutical composition, wherein the volume comprises: (a) about 250 mg enoblituzumab; (b) about 2.7 mg glacial acetic acid; (c) about 7.4 mg sodium acetate trihydrate; (d) about 900 mg sucrose; (e) about 1 mg PS80; and (f) water; and wherein the composition has a pH of about 4.6 to about 5.4.

The invention provides an embodiment of such containers, wherein the container comprises about 10 mL volume of the pharmaceutical composition, wherein the volume comprises: (a) about 1,200 mg enoblituzumab; (b) about 20 mM sodium acetate; (c) about 900 mg sucrose; (d) about 1 mg PS80; and (e) water; and wherein the composition has a pH of about 4.4 to about 5.2.

The invention provides an embodiment of such containers, wherein the container comprises about 10 mL volume of the pharmaceutical composition, wherein the volume comprises: (a) about 1,200 mg enoblituzumab; (b) about 5.2 mg glacial acetic acid (c) about 15 mg sodium acetate trihydrate; (d) about 900 mg sucrose; (e) about 1 mg PS80; and (f) water; and wherein the composition has a pH of about 4.4 to about 5.2.

The invention provides an embodiment of such containers, wherein the container comprises about 17 mL volume of the pharmaceutical composition, wherein the volume comprises: (a) about 425 mg enoblituzumab; (b) about 10 mM; (c) about 1530 mg sucrose; (d) about 1.7 mg PS80; and (e) water; and wherein the composition has a pH of about 4.6 to about 5.4.

The invention provides an embodiment of such containers, wherein the container comprises about 17 mL volume of the pharmaceutical composition, wherein the volume comprises: (a) about 425 mg enoblituzumab; (b) about 3.06 mg glacial acetic acid (c) about 16.15 mg sodium acetate trihydrate; (d) about 1530 mg sucrose; (e) about 1.7 mg PS80; and (f) water; and wherein the composition has a pH of about 4.7 to about 5.5.

The invention provides an embodiment of such containers, wherein the container comprises about 17 mL volume of the pharmaceutical composition, wherein the volume comprises: (a) about 425 mg enoblituzumab; (b) about 4.59 mg glacial acetic acid; (c) about 12.58 mg sodium acetate trihydrate; (d) about 1530 mg sucrose; (e) about 1.7 mg PS80; and (f) water; and wherein the composition has a pH of about 4.6 to about 5.4.

The invention additionally provides a sealed package comprising any of the pharmaceutical compositions disclosed herein, or any of the containers disclosed herein.

The invention additional provides a kit comprising any of the pharmaceutical compositions disclosed herein, any of the containers disclosed herein, or any of the sealed packages disclosed herein, and optionally further comprising instructions for administration of the pharmaceutical composition to a subject in need thereof.

The invention additionally provides a sealed package comprising any of the pharmaceutical compositions disclosed herein, or any of the containers disclosed herein, or any of the kits disclosed herein, and optionally further comprising instructions for administration of the pharmaceutical composition to a subject in need thereof.

The invention additionally provides a method of treating cancer, comprising administering enoblituzumab to a subject in need thereof using any of the pharmaceutical compositions disclosed herein, or any of the containers disclosed herein, or any of the sealed packages disclosed herein, or any of the kits disclosed herein.

The disclosure additionally provides a method of treating cancer, comprising administering retifanlimab to a subject in need thereof using any of the pharmaceutical compositions disclosed herein, any of the containers disclosed herein, any of the sealed packages disclosed herein, or any of the kits disclosed herein, wherein the method comprises:
a) diluting the pharmaceutical composition in a container comprising 0.9% sodium chloride or D5W, to obtain a dosing solution;
b) inverting the container to mix the diluted solution; and
c) attaching the container containing the dosing solution to a device for administration to the subject.

The invention provides an embodiment of such methods, wherein the container is an IV bag or a syringe containing 0.9% sodium chloride.

The invention provides an embodiment of such methods, wherein the container is an IV bag or a syringe containing D5W.

The invention additionally provides the use of any of the pharmaceutical compositions disclosed herein, for the production of a medicament for the treatment of cancer in a subject in need thereof.

The invention additionally provides the use of any of the pharmaceutical compositions disclosed herein, or any of the containers disclosed herein, or any of the sealed packages disclosed herein, or any of the kits disclosed herein, for the treatment of cancer in a subject in need thereof.

The invention additionally provides the use of any of the pharmaceutical compositions disclosed herein, any of the containers disclosed herein, any of the sealed packages disclosed herein, or any of the kits disclosed herein, for the treatment of cancer in a subject in need thereof, wherein the use comprises:
a) diluting the pharmaceutical composition in a container comprising 0.9% sodium chloride or D5W, to obtain a dosing solution;
b) inverting the container to mix the diluted solution; and
c) attaching the container containing the dosing solution to a device for administration to the subject.

The invention provides an embodiment of such uses, wherein the container is an IV bag or a syringe containing 0.9% sodium chloride.

The invention provides an embodiment of such uses, wherein the container is an IV bag or a syringe containing D5W.

The invention provides an embodiment of the methods, or uses of the present disclosure, wherein the dosing solution maintains monomeric purity of the enoblituzumab for about 6 hours at about 25° C. or for about 24 hours at about 2° C. to about 8° C.

The invention provides an embodiment of the methods or uses of the present disclosure, wherein the administration is by IV infusion for at least about 30 minutes. The invention further provides an embodiment of the methods, or uses of the present disclosure, wherein the administration is by continuous infusion for at least about 60 minutes. The invention further provides an embodiment of the methods, or uses of the present disclosure, wherein the administration is by IV infusion for at least about 120 minutes.

The invention provides an embodiment of the methods or uses of the present disclosure, wherein the pharmaceutical composition is diluted to obtain a weight-based treatment dose of about 6 mg/kg to about 15 mg/kg.

The invention provides an embodiment of the methods or uses of the present disclosure, wherein the pharmaceutical composition is diluted to obtain a weight-based treatment dose of about 15 mg/kg

The invention provides an embodiment of the methods or uses of the present disclosure, wherein administration of the dosing solution is once every 3 weeks.

The invention provides an embodiment of the methods or uses of the present disclosure, wherein the cancer expresses B7-H3.

The invention provides an embodiment of the methods or uses of the present disclosure, wherein the cancer is selected from the group consisting of: adrenal gland cancer, AIDS-associated cancer, alveolar soft part sarcoma, anal cancer, squamous cell carcinoma of the anal canal (SCAC), bladder cancer, bone cancer, brain and spinal cord cancer, breast cancer, HER2⁺ breast cancer, Triple-Negative Breast Cancer (TNBC), carotid body tumor, cervical cancer, HPV-related cervical cancer, chondrosarcoma, chordoma, chromophobe renal cell carcinoma, clear cell carcinoma, colon cancer, colorectal cancer, desmoplastic small round cell tumor, ependymoma, endometrial cancer, unselected endometrial cancer, MSI-high endometrial cancer, dMMR endometrial cancer, POLE exonuclease domain mutation positive endometrial cancer, Ewing's sarcoma, extraskeletal myxoid chondrosarcoma, gallbladder cancer, bile duct cancer, cholangiocarcinoma bile duct cancer, gastric cancer, gastroesophageal junction (GEJ) cancer, gestational trophoblastic disease, germ cell tumor, glioblastoma, head and neck cancer, squamous cell carcinoma of head and neck (SCCHN), a hematological malignancy, a hepatocellular carcinoma, islet cell tumor, Kaposi's Sarcoma, kidney cancer, leukemia, acute myeloid leukemia, liposarcoma/malignant lipomatous tumor, liver cancer, hepatocellular carcinoma liver cancer (HCC), lymphoma, diffuse large B-cell lymphoma (DLBCL), non-Hodgkin's lymphoma (NHL), lung cancer, small cell lung cancer (SCLC), non-small cell lung cancer (NSCLC), medulloblastoma, melanoma, uveal melanoma, meningioma, Merkel cell carcinoma, mesothelioma, mesothelial pharyngeal cancer, multiple endocrine neoplasia, multiple myeloma, myelodysplastic syndrome, neuroblastoma, neuroendocrine tumors, ovarian cancer, pancreatic cancer, papillary thyroid carcinoma, parathyroid tumor, pediatric cancer, peripheral nerve sheath tumor, pharyngeal cancer, pheochromocytoma, pituitary tumor, prostate cancer, metastatic castration resistant prostate cancer (mCRPC), posterious uveal melanoma, renal metastatic cancer, rhabdoid tumor, rhabdomyosarcoma, sarcoma, skin cancer, a small round blue cell tumor of childhood, neuroblastoma, rhabdomyosarcoma, soft-tissue sarcoma, squamous cell cancer, stomach cancer, synovial sarcoma, testicular cancer, thymic carcinoma, thymoma, thyroid cancer, urothelial cancer, and uterine cancer.

The invention provides an embodiment of the methods or uses of the present disclosure, wherein the cancer is anal cancer, bladder cancer, breast cancer, bile duct cancer, cervical cancer, colorectal cancer, endometrial cancer, gastric cancer, GEJ cancer, head and neck cancer, liver cancer, lung cancer, lymphoma, ovarian cancer, prostate cancer, skin cancer, and urothelial cancer.

The invention provides an embodiment of the methods or uses of the present disclosure, wherein the anal cancer is SCAC, the lung cancer is NSCLC, the breast cancer is TNBC, the skin cancer is melanoma or Merkel cell carcinoma, the head and neck cancer is SCCHN, the prostate cancer is mCRPC. The invention provides an embodiment of the methods, or uses of the present disclosure, wherein the subject is a human subject.

### DETAILED DESCRIPTION OF THE INVENTION

The present invention provides pharmaceutical compositions, for storage and administration, comprising an anti-human B7-H3 (“hB7-H3”) antibody (“enoblituzumab”) and buffering agents. The invention further provides containers and kits comprising such pharmaceutical compositions. The invention further provides the use of such pharmaceutical compositions, containers, and kits containing enoblituzumab for the treatment of a cancer, and in certain aspects, treatment of a cancer expressing B7-H3, for example with a therapeutically effective amount or prophylactically effective amount of enoblituzumab.

### L. Enoblituzumab

Enoblituzumab (also known as enoblituzumab; CAS Reg No. 1353485-38-7, see e.g., U.S. Pat. No. 8,802,093) is an Fc-optimized monoclonal antibody that binds to B7-H3 and mediates enhanced ADCC activity. Enoblituzumab contains a human IgG1 Fc region containing L235V, F243L, R292P, Y300L, and P396L substitutions to enhance ADCC activity, wherein the numbering of the residues in an IgG heavy chain is that of the EU index as in Kabat et al., SEQUENCES OF PROTEINS OF IMMUNOLOGICAL INTEREST, 5^{th} Ed. Public Health Service, NH1, MD (1991), and refers to the numbering of the human IgG1 EU antibody. The amino acid sequences of the heavy and light chains of enoblituzumab are presented below (WHO Drug Information 2016, Recommended INN: List 76, 30(3)):496). The CDRs as defined by Kabat are underlined.

The amino acid sequence of the Heavy Chain of Enoblituzumab is (SEQ ID NO:1) (CDRH residues are shown bolded and underlined; the constant region is shown with double underline and Fc region substitutions are shown bolded and double underlined):

EVQLVESGGG LVQPGGSLRL SCAASGFTFS SFGMHWVRQA
PGKGLEWVAY ISSDSSAIYY ADTVKGRFTI SRDNAKNSLY
LQMNSLRDED TAVYYCGRGR ENIYYGSRLD YWGQGTTVTV
SSASTKGPSV FPLAPSSKST SGGTAALGCL VKDYFPEPVT
VSWNSGALTS GVHTFPAVLQ SSGLYSLSSV VTVPSSSLGT
QTYICNVNHK PSNTKVDKRV EPKSCDKTHT CPPCPAPEL [symbol]
GGPSVFL [symbol] PP KPKDTLMISR TPEVTCVVVD VSHEDPEVKF
NWYVDGVEVH NAKTKP [symbol] EEQ YNST [symbol] RVVSV LTVLHQDWLN
GKEYKCKVSN KALPAPIEKT ISKAKGQPRE PQVYTLPPSR
EEMTKNQVSL TCLVKGFYPS DIAVEWESNG QPENNYKTTP [symbol]
VLDSDGSFF LYSKLTVDKS RWQQGNVFSC SVMHEALHNH
YTQKSLSLSP GK

The amino acid sequence of the Light Chain of Enoblituzumab is (SEQ ID NO:2) (CDRL residues are shown bolded and underlined; the constant region is shown with double underline):

DIQLTQSPSF LSASVGDRVT ITCKASQNVD TNVAWYQQKP
GKAPKALIYS ASYRYSGVPS RFSGSGSGTD FTLTISSLQP
EDFATYYCQQ YNNYPFTFGQ GTKLEIKRTV AAPSVFIFPP
SDEQLKSGTA SVVCLLNNFY PREAKVQWKV DNALQSGNSQ
ESVTEQDSKD STYSLSSTLT LSKADYEKHK VYACEVTHQG
LSSPVTKSFN RGEC

### II. Pharmaceutical Compositions

The pharmaceutical compositions of the invention comprise enoblituzumab, buffering agents and stabilizers, and are also referred herein as “enoblituzumab compositions” or “enoblituzumab drug product (DP) compositions”.

As used herein, “about” will be understood by persons of ordinary skill in the art and will vary to some extent depending upon the context in which it is used. If there are uses of the term which are not clear to persons of ordinary skill in the art and which are not otherwise defined herein, given the context in which it is used, “about” will mean up to plus or minus 10% of the particular term.

As used herein, the singular forms “a,” “an” and “the” include plural referents unless the context clearly dictates otherwise. Thus, for example, reference to “a” component includes aspects having two or more such components, unless the context clearly indicates otherwise. Also, the word “or” when used without a preceding “either” (or other similar language indicating that “or” is unequivocally meant to be exclusive—e.g., only one of x or y, etc.) shall be interpreted to be inclusive (e.g., “x or y” means one or both x or y).

The term “and/or” shall also be interpreted to be inclusive (e.g., “x and/or y” means one or both x or y). In situations where “and/or” or “or” are used as a conjunction for a group of three or more items, the group should be interpreted to include one item alone, all the items together, or any combination or number of the items. Moreover, terms used in the specification and claims such as have, having, include, and including should be construed to be synonymous with the terms comprise and comprising. Other elements may optionally be present other than the elements specifically identified by the “and/or” clause, whether related or unrelated to those elements specifically identified. As a non-limiting example, a reference to “X and/or Y” may refer, in one embodiment, to X only (optionally including elements other than Y); in some embodiments, to Y only (optionally including elements other than X); in yet some embodiments, to both X and Y (optionally including other elements).

As used herein, “acetate” refers to the acetate component of a pharmaceutical composition. For example, the acetate component can be made up of acetic acid, acetate salts, and/or an acetate buffer.

As used herein, the term “aqueous” refers to a water-containing solution.

As used herein, the term “stable” refers to enoblituzumab substantially retaining its physical stability, chemical stability, pharmaceutical activity and/or its biological activity upon storage.

The term “shelf-life” refers to the period of time during which the pharmaceutical compositions can be stored, in which physical stability, chemical stability, pharmaceutical activity and/or biological activity are/is substantially retained.

As will be understood by one skilled in the art, for any and all purposes, particularly in terms of providing a written description, all ranges disclosed herein also encompass any and all possible subranges and combinations of subranges thereof, inclusive of the endpoints. As such, all disclosed ranges are to be understood to encompass and provide support for claims that recite any and all subranges or any and all individual values subsumed by each range. For example, a stated range of 1 to 10 should be considered to include and provide support for claims that recite any and all subranges or individual values that are between and/or inclusive of the minimum value of 1 and the maximum value of 10; that is, all subranges beginning with a minimum value of 1 or more and ending with a maximum value of 10 or less (e.g., 5.5 to 10, 2.34 to 3.56, and so forth) or any values from 1 to 10 (e.g., 3, 5.8, 9.9994, and so forth).

Any listed range may be easily recognized as sufficiently describing and enabling the same range being broken down into at least equal halves, thirds, quarters, fifths, tenths, etc. As a non-limiting example, each range discussed herein may be readily broken down into a lower third, middle third and upper third, etc. As will also be understood by one skilled in the art all language such as “up to,” “at least,” “greater than,” “less than,” and the like, include the number recited and refer to ranges which may be subsequently broken down into subranges as discussed herein. Further, as will be understood by one skilled in the art, a range includes each individual member. Thus, for example, a group having 1-3 layers refers to groups having 1, 2, or 3 layers. Similarly, a group having 1-5 layers refers to groups having 1, 2, 3, 4, or 5 layers, and so forth.

The embodiments illustratively disclosed herein may suitably be practiced in the absence of any element or elements, limitation or limitations not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

Definitions that are contained in text incorporated by reference are excluded to the extent that they contradict definitions in this disclosure.

The present disclosure provides pharmaceutical compositions that substantially retain the physical and chemical stability, of enoblituzumab as well as its pharmaceutical activity and/or biological activity upon storage. In one embodiment, about 90% or more, about 80% or more, about 70% or more, or about 60% or more of the physical stability, chemical stability, pharmaceutical activity and/or biological activity of enoblituzumab is retained during storage of a pharmaceutical composition of the disclosure. In one embodiment, during the shelf-life time period, about 90% or more, about 85% or more, about 80% or more, about 75% or more, about 70% or more, about 65% or more, or about 60% or more of the physical stability, chemical stability, pharmaceutical activity and/or biological activity of enoblituzumab is retained. The shelf-life of a pharmaceutical composition is generally selected based on the period of time a molecule is stable in such composition.

In one embodiment, the shelf-life of a pharmaceutical composition of the invention is at least about 1 month at about 25° C., at least about 2 months at about 25° C., at least about 3 months at about 25° C., at least about 4 months at about 25° C., at least about 6 months at about 25° C., at least about 6 months at 2-8° C., at least about 12 months at 2-8° C., at least about 18 months at 2-8° C., at least about 24 months at 2-8° C., at least about 30 months at 2-8° C., at least about 36 months at 2-8° C., at least about 48 months at 2-8° C., or more than about 48 months at 2-8° C. In another embodiment, the shelf-life of a pharmaceutical composition of the invention is at least about 6 months at about 25° C. In another embodiment, the shelf-life of a pharmaceutical composition of the invention is at least about 24 months at 2-8° C. In another embodiment, the shelf-life of a pharmaceutical composition of the invention is at least about 36 months at 2-8° C. In another embodiment, the shelf-life of a pharmaceutical composition of the invention is at least about 48 months at 2-8° C.

One measure of physical and chemical stability is the monomeric purity of enoblituzumab in pharmaceutical compositions of the invention or in dosing solutions of the invention. The monomeric purity of enoblituzumab can be determined by evaluating the amount of protein in such composition or solution having the expected molecule weight (monomeric enoblituzumab), species with a molecular weight greater than the monomer (HMW species), and/or species having a molecular weight lower than the monomer (LMW species) by any suitable method. Thus, the loss of monomeric purity can be measured by determining the loss of enoblituzumab protein having the expected molecule weight (monomeric enoblituzumab), and/or the accumulation of HMW, and/or LMW species after the indicated period of time. In certain embodiments, the percent (%) of each species (monomer, HMW, and LMW) is calculated as the percent (%) of the total protein. In one embodiment, the loss of monomeric purity of enoblituzumab in a pharmaceutical composition of the invention or in a dosing solution of the invention is about 15% or less, or about 10% or less, or about 5% or less, or about 4% or less, or about 3% or less, or about 2% or less, or about 1% or less, over the indicated period of time. In one embodiment, the loss of monomeric purity of enoblituzumab in a pharmaceutical composition of the invention or in a dosing solution of the invention is about 6% or less over the indicated period of time. In one embodiment, the loss of monomeric purity of enoblituzumab in a pharmaceutical composition of the invention or in a dosing solution of the invention is about 5% or less over the indicated period of time. In another embodiment, the loss of monomeric purity of enoblituzumab in a pharmaceutical composition of the invention or in a dosing solution of the invention is less than about 4% over the indicated period of time. In another embodiment, the loss of monomeric purity of enoblituzumab in a pharmaceutical composition of the invention or in a dosing solution of the invention is about 3% or less over the indicated period of time. In another embodiment, the loss of monomeric purity of enoblituzumab in a pharmaceutical composition of the invention or in a dosing solution of the invention is about 2% or less over the indicated period of time. In certain embodiments, the HMW and/or LMW species of the enoblituzumab in a composition of the invention or in a dosing solution of the invention is/are measured via size exclusion high performance liquid chromatography (SE-HPLC). In one such embodiment, the percent (%) of each species is calculated as the area of the SE-HPLC species peak (i.e., monomer, HMW, LMW), divided by the sum of all peaks, the percent (%) of the total protein.

In other embodiments, the monomeric purity of enoblituzumab in a pharmaceutical composition of the invention is maintained for at least about 1 month at about 25° C., at least about 2 months at about 25° C., at least about 3 months at about 25° C., at least about 4 months at about 25° C., at least about 6 months at about 25° C., at least about 6 months at about 2° C. to about 8° C., at least about 12 months at about 2° C. to about 8° C., at least about 18 months at about 2° C. to about 8° C., at least about 24 months at about 2° C. to about 8° C., at least about 30 months about 2° C. to about 8° C., at least about 36 months about 2° C. to about 8° C., at least about 48 months, or more than about 48 months at about 2° C. to about 8° C. In one embodiment, monomeric purity of enoblituzumab in a pharmaceutical composition of the invention is maintained at least about 6 months at about 25° C. In another embodiment, monomeric purity of enoblituzumab in a pharmaceutical composition of the invention is maintained for about 36 months or more at about 2° C. to about 8° C. In another embodiment, the monomeric purity of enoblituzumab in a pharmaceutical composition of the invention is maintained for about 48 months at about 2° C. to about 8° C.

Another measure of stability is the stability of the charge heterogeneity profile of enoblituzumab in pharmaceutical compositions of the invention or in dosing solutions of the invention. Protein compositions may comprise a variety of variants that differ in their isoelectric point (pI). Such variants are referred to as charge variants. Thus, the heterogeneity profile can be determined by measuring the main charge peak (MCP), the acidic variants (AV), and the basic variants (BV) by any suitable method. For example, a enoblituzumab composition of the invention can comprise MCP, AV and BV components, and changes to the heterogeneity profile may be measured by determining the loss of the MCP and/or the accumulation of AV, and/or BV after the indicated time. In one embodiment, the decrease in the MCP of enoblituzumab in a pharmaceutical composition of the invention or in a dosing solution of the invention is about 15% or less, or about 10% or less, or about 5% or less, or about 4% or less, or about 3% or less, or about 2% or less, or about 1% or less, over the indicated period of time. In one embodiment, the increase in the AV of enoblituzumab in a pharmaceutical composition of the invention or in a dosing solution of the invention is about 15% or less, or about 10% or less, or about 5% or less, or about 4% or less, or about 3% or less, or about 2% or less, or about 1% or less, over the indicated period of time. In another embodiment, the increase in the BV of enoblituzumab in a pharmaceutical composition of the invention or in a dosing solution of the invention is about 15% or less, or about 10% or less, or about 5% or less, or about 4% or less, or about 3% or less, or about 2% or less, or about 1% or less, over the indicated period of time. In another embodiment, the decrease in the MCP of enoblituzumab in a pharmaceutical composition of the invention or in a dosing solution of the invention is about 7% or less over the indicated period of time. In another embodiment, the decrease in the MCP of enoblituzumab in a pharmaceutical composition of the invention or in a dosing solution of the invention is about 6% or less over the indicated period of time. In another embodiment, the decrease in the MCP of enoblituzumab in a pharmaceutical composition of the invention or in a dosing solution of the invention is about 5% or less over the indicated period of time. In another embodiment, the increase in the AV of enoblituzumab in a pharmaceutical composition of the invention or in a dosing solution of the invention is about 7% or less over the indicated period of time. In another embodiment, the increase in the AV of enoblituzumab in a pharmaceutical composition of the invention or in a dosing solution of the invention is about 6% or less over the indicated period of time. In another embodiment, the increase in the AV of enoblituzumab in a pharmaceutical composition of the invention or in a dosing solution of the invention is about 5% or less over the indicated period of time. In another embodiment, the increase in the BV of enoblituzumab in a pharmaceutical composition of the invention or in a dosing solution of the invention is about 4% or less over the indicated period of time. In another embodiment, the increase in the BV of enoblituzumab in a pharmaceutical composition of the invention or in a dosing solution of the invention is about 3% or less over the indicated period of time. In another embodiment, the increase in the BV of enoblituzumab in a pharmaceutical composition of the invention or in a dosing solution of the invention is about 2% or less over the indicated period of time. In certain embodiments, the MCP, AV, and BV of the enoblituzumab in the pharmaceutical composition of the invention or the dosing solution of the invention is measured via by ion exchange high performance liquid chromatography (IE-HPLC). In certain embodiments, the MCP, AV, and BV of the enoblituzumab in the pharmaceutical composition of the invention or the dosing solution of the invention is measured via by capillary isoelectric focusing (cIEF).

In other embodiments, the heterogeneity profile of enoblituzumab in a pharmaceutical composition of the invention is maintained for at least about 1 month at about 25° C., at least about 2 months at about 25° C., at least about 3 months at about 25° C., at least about 4 months at about 25° C., at least about 6 months at about 25° C., at least about 6 months at about 2° C. to about 8° C., at least about 12 months at about 2° C. to about 8° C., at least about 18 months at about 2° C. to about 8° C., at least about 24 months at about 2° C. to about 8° C., at least about 30 months about 2° C. to about 8° C., at least about 36 months about 2 to about 8° C., at least about 48 months, or more than about 48 months at about 2° C. to about 8° C. In one embodiment, heterogeneity profile of enoblituzumab in a pharmaceutical composition of the invention is maintained at least about 6 months at about 25° C. In another embodiment, heterogeneity profile of enoblituzumab in a pharmaceutical composition of the invention is maintained for about 36 months or more at about 2° C. to about 8° C. In another embodiment, the heterogeneity profile of enoblituzumab in a pharmaceutical composition of the invention is maintained for about 48 months at about 2° C. to about 8° C.

### A. Enoblituzumab Compositions

The components of the pharmaceutical compositions (i.e., enoblituzumab compositions) of the invention can be supplied mixed together in unit dosage form, for example, as a liquid composition, in a hermetically sealed container such as a vial, ampoule, or sachet indicating the quantity of active agent. In one embodiment, the pharmaceutical composition of the invention is supplied as a liquid solution. Such liquid solutions can be stored at between about 2° C. and about 8° C. in their original containers until ready to be administered, although such liquid solutions may be stored at room temperature (about 25° C.) for short periods prior to administration.

In certain embodiments, where an enoblituzumab composition of the invention is to be administered by infusion, it can be dispensed, for example, with a syringe, container, bag, or infusion bottle containing sterile 0.9% sodium chloride (e.g., normal saline). In certain embodiments, where an enoblituzumab composition of the invention is administered by injection, 0.9% sodium chloride can be provided so that the ingredients may be mixed prior to administration as detailed herein. Such enoblituzumab compositions can comprise a prophylactically or therapeutically effective amount of enoblituzumab.

In certain embodiments where a enoblituzumab composition of the invention is to be administered by infusion, it can be dispensed, for example, with a syringe, a container, bag, or infusion bottle containing sterile 5% dextrose in water (“D5W”). In certain embodiments where a enoblituzumab composition of the disclosure is administered by injection, D5W can be provided so that the ingredients can be mixed prior to administration as detailed herein. Such enoblituzumab compositions can comprise a prophylactically or therapeutically effective amount of enoblituzumab.

In one embodiment, a pharmaceutical composition of the invention comprises enoblituzumab, acetate, sucrose, PS80 and water. In certain embodiments, the pharmaceutical compositions of the invention do not comprise an antioxidant (e.g., methionine).

The acetate component can be made up of acetic acid and an acetate salt. Acceptable acetate salts include, but are not limited to: calcium acetate, magnesium acetate, potassium acetate, sodium acetate, and zinc acetate. In one embodiment, the acetate comprises glacial acetic acid, and sodium acetate.

In one embodiment, the pharmaceutical composition of the invention comprises enoblituzumab at a concentration of about 5 mg/mL to about 200 mg/mL. In another embodiment, the pharmaceutical composition of the invention comprises enoblituzumab at a concentration of about 5 mg/mL to about 60 mg/mL. In another embodiment, the pharmaceutical composition of the invention comprises enoblituzumab at a concentration of about 20 mg/mL to about 130 mg/mL. In another embodiment, the pharmaceutical composition of the invention comprises enoblituzumab at a concentration of about 20 mg/mL to about 40 mg/mL. In another embodiment, the pharmaceutical composition of the invention comprises enoblituzumab at a concentration of about 20 mg/mL to about 30 mg/mL. In another embodiment, the pharmaceutical composition of the invention comprises enoblituzumab at a concentration of about 22.5 mg/mL to about 27.5 mg/mL. In another embodiment, the pharmaceutical composition of the invention comprises enoblituzumab at a concentration of about 60 mg/mL to about 130 mg/mL. In another embodiment, the pharmaceutical composition of the invention comprises enoblituzumab at a concentration of about 85 mg/mL to about 105 mg/mL. In another embodiment, the pharmaceutical composition of the invention comprises enoblituzumab at a concentration of about 115 mg/mL to about 125 mg/mL. In another embodiment, the pharmaceutical composition of the invention comprises enoblituzumab at a concentration of about 135 mg/mL to about 155 mg/mL. In another embodiment, the pharmaceutical composition of the invention comprises enoblituzumab at a concentration of about 165 mg/mL to about 175 mg/mL. In another embodiment, the pharmaceutical composition of the invention comprises enoblituzumab at a concentration of about 185 mg/mL to about 200 mg/mL. In another embodiment, the pharmaceutical composition of the invention comprises enoblituzumab at a concentration of about 25 mg/mL. In another embodiment, the pharmaceutical composition of the invention comprises enoblituzumab at a concentration of about 60 mg/mL. In another embodiment, the pharmaceutical composition of the invention comprises enoblituzumab at a concentration of about 90 mg/mL. In another embodiment, the pharmaceutical composition of the invention comprises enoblituzumab at a concentration of about 120 mg/mL. Also contemplated are enoblituzumab concentrations between any of these values, such as about 10 mg/mL about 22 mg/mL, about 27 mg/mL, about 30 mg/mL, about 40 mg/mL, about 50 mg/mL, about 80 mg/mL, about 100 mg/mL, about 150 mg/mL, etc.

In one embodiment, the pharmaceutical composition of the invention comprises about 5 mM to about 30 mM acetate. In another embodiment, the pharmaceutical composition of the invention comprises about 5 mM to about 25 mM acetate. In another embodiment, the pharmaceutical composition of the invention comprises about 8 mM to about 24 mM acetate. In another embodiment, the pharmaceutical composition of the invention comprises about 7.5 mM to about 15 mM acetate. In another embodiment, the pharmaceutical composition of the invention comprises about 8 mM to about 12 mM acetate In another embodiment, the pharmaceutical composition of the invention comprises about 9 mM to about 11 mM acetate. In one embodiment, the pharmaceutical composition of the invention comprises about 16 mM to about 24 mM acetate. In one embodiment, the pharmaceutical composition of the invention comprises about 18 mM to about 22 mM acetate. In another embodiment, the pharmaceutical composition of the invention comprises about 10 mM acetate. In another embodiment, the pharmaceutical composition of the invention comprises about 20 mM acetate. Also contemplated are acetate concentrations between any of these values, such as about 8 mM, about 14 mM, about 18 mM, etc. In one embodiment, the acetate in the composition of the invention comprises glacial acetic acid and sodium acetate (e.g., sodium acetate anhydrous, sodium acetate monohydrate, and/or sodium acetate trihydrate). It is appreciated that sodium acetate monohydrate and/or sodium acetate anhydrous and/or sodium acetate trihydrate may be used in combination with glacial acetic acid to obtain the desired acetate concentration. As provided herein, alternative forms of acetate can be used in place of sodium acetate in the acetate buffer, including but not limited to, magnesium acetate, potassium acetate, calcium acetate, and zinc acetate.

In one embodiment, the pharmaceutical composition of the invention comprises glacial acetic acid at a concentration of about 0.05 mg/mL to about 0.8 mg/mL. In another embodiment, the pharmaceutical composition of the invention comprises glacial acetic acid at a concentration of about 0.1 mg/mL to about 0.65 mg/mL. In another embodiment, the pharmaceutical composition of the invention comprises glacial acetic acid at a concentration of about 0.1 mg/mL to about 0.35 mg/mL. In another embodiment, the pharmaceutical composition of the invention comprises glacial acetic acid at a concentration of about 0.16 mg/mL to about 0.20 mg/mL. In another embodiment, the pharmaceutical composition of the invention comprises glacial acetic acid at a concentration of about 0.23 mg/mL to about 0.30 mg/mL. In another embodiment, the pharmaceutical composition of the invention comprises glacial acetic acid at a concentration of about 0.4 mg/mL to about 0.65 mg/mL. In another embodiment, the pharmaceutical composition of the invention comprises glacial acetic acid at a concentration of about 0.45 mg/mL to about 0.57 mg/mL. In another embodiment, the pharmaceutical composition of the invention comprises glacial acetic acid at a concentration of about 0.18 mg/mL. In another embodiment, the pharmaceutical composition of the invention comprises glacial acetic acid at a concentration of about 0.27 mg/mL. In another embodiment, the pharmaceutical composition of the invention comprises glacial acetic acid at a concentration of about 0.52 mg/mL. Also contemplated are glacial acetic acid concentrations between any of these values, such as about 0.08 mg/mL, 0.15 mg/mL, 0.25 mg/mL, etc.

In one embodiment, the pharmaceutical composition of the invention comprises sodium acetate trihydrate at a concentration of about 0.50 mg/mL to about 2.0 mg/mL. In another embodiment, the pharmaceutical composition of the invention comprises sodium acetate trihydrate at a concentration of about 0.6 mg/mL to about 1.8 mg/mL. In another embodiment, the pharmaceutical composition of the invention comprises sodium acetate trihydrate at a concentration of about 0.6 mg/mL to about 1.2 mg/mL. In another embodiment, the pharmaceutical composition of the invention comprises sodium acetate trihydrate at a concentration of about 0.86 mg/mL to about 1.1 mg/mL. In another embodiment, the pharmaceutical composition of the invention comprises sodium acetate trihydrate at a concentration of about 0.66 mg/mL to about 0.81 mg/mL. In another embodiment, the pharmaceutical composition of the invention comprises sodium acetate trihydrate at a concentration of about 1.2 mg/mL to about 1.8 mg/mL. In another embodiment, the pharmaceutical composition of the invention comprises sodium acetate trihydrate at a concentration of about 1.35 mg/mL to about 1.65 mg/mL. In another embodiment, the pharmaceutical composition of the invention comprises sodium acetate trihydrate at a concentration of about 0.74 mg/mL. In another embodiment, the pharmaceutical composition of the invention comprises sodium acetate trihydrate at a concentration of about 0.95 mg/mL. In another embodiment, the pharmaceutical composition of the invention comprises sodium acetate trihydrate at a concentration of about 1.5 mg/mL. Also contemplated are sodium acetate trihydrate concentrations between any of these values, such as about 0.9 mg/mL, about 1.2 mg/mL, about 1.7 mg/mL, etc.

In one embodiment, the pharmaceutical composition of the invention comprises glacial acetic acid at a concentration of about 0.05 mg/mL to about 0.8 mg/mL and sodium acetate trihydrate at a concentration of about 0.50 mg/mL to about 2.0 mg/mL. In another embodiment, the pharmaceutical composition of the invention comprises glacial acetic acid at a concentration of about 0.1 mg/mL to about 0.65 mg/mL and sodium acetate trihydrate at a concentration of about 0.6 mg/mL to about 1.8 mg/mL. In another embodiment, the pharmaceutical composition of the invention comprises glacial acetic acid at a concentration of about 0.1 mg/mL to about 0.35 mg/mL and sodium acetate trihydrate at a concentration of about 0.6 mg/mL to about 1.2 mg/mL. In another embodiment, the pharmaceutical composition of the invention comprises glacial acetic acid at a concentration of about 0.16 mg/mL to about 0.20 mg/mL and sodium acetate trihydrate at a concentration of about 0.86 mg/mL to about 1.1 mg/mL. In another embodiment, the pharmaceutical composition of the invention comprises glacial acetic acid at a concentration of about 0.23 mg/mL to about 0.30 mg/mL and sodium acetate trihydrate at a concentration of about 0.66 mg/mL to about 0.81 mg/mL. In another embodiment, the pharmaceutical composition of the invention comprises glacial acetic acid at a concentration of about 0.4 mg/mL to about 0.65 mg/mL and sodium acetate trihydrate at a concentration of about 1.2 mg/mL to about 1.8 mg/mL. In another embodiment, the pharmaceutical composition of the invention comprises glacial acetic acid at a concentration of about 0.45 mg/mL to about 0.57 mg/mL and sodium acetate trihydrate at a concentration of about 1.35 mg/mL to about 1.65 mg/mL. In another embodiment, the pharmaceutical composition of the invention comprises glacial acetic acid at a concentration of about 0.27 mg/mL and sodium acetate trihydrate at a concentration of about 0.74 mg/mL. In another embodiment, the pharmaceutical composition of the invention comprises glacial acetic acid at a concentration of about 0.18 mg/mL and sodium acetate trihydrate at a concentration of about 0.95 mg/mL. In another embodiment, the pharmaceutical composition of the invention comprises glacial acetic acid at a concentration of about 0.52 mg/mL and sodium acetate trihydrate at a concentration of about 1.5 mg/mL.

In one embodiment, the pharmaceutical composition of the invention comprises sucrose at a concentration of about 50 mg/mL to about 130 mg/mL sucrose. In another embodiment, the pharmaceutical composition of the invention comprises sucrose at a concentration of about 72 mg/mL to about 108 mg/mL. In another embodiment, the pharmaceutical composition of the invention comprises sucrose at a concentration of about 76 mg/mL to about 104 mg/mL. In another embodiment, the pharmaceutical composition of the invention comprises sucrose at a concentration of about 80 mg/mL to about 100 mg/mL. In another embodiment, the pharmaceutical composition of the invention comprises sucrose at a concentration of about 90 mg/mL. Also contemplated are sucrose concentrations between any of these values, such as about 85 mg/mL, about 87 mg/mL, about 92 mg/mL, etc.

In one embodiment, the pharmaceutical composition of the invention comprises PS80 at a concentration of about 0.05 mg/mL to about 0.6 mg/mL. In another embodiment, the pharmaceutical composition of the invention comprises PS80 at a concentration of about 0.08 mg/mL to about 0.53 mg/mL. In another embodiment, the pharmaceutical composition of the invention comprises PS80 at a concentration of about 0.08 mg/mL to about 0.2 mg/mL. In another embodiment, the pharmaceutical composition of the invention comprises PS80 at a concentration of about 0.08 mg/mL to about 0.12 mg/mL. In another embodiment, the pharmaceutical composition of the invention comprises PS80 at a concentration about 0.1 mg/mL. Also contemplated are PS80 concentrations between any of these values, such as about 0.09 mg/mL, about 0.11 mg/mL, about 0.13 mg/mL, etc.

In one embodiment, the pharmaceutical composition of the invention has a pH of about 4.0 to about 6.0. In another embodiment, the pharmaceutical composition of the invention has a pH of about 4.4 to about 5.6. In another embodiment, the pharmaceutical composition of the invention has a pH of about 4.3 to about 5.3. In another embodiment, the pharmaceutical composition of the invention has a pH of about 4.5 to about 5.5. In another embodiment, the pharmaceutical composition of the invention has a pH of about 4.7 to about 5.5 (i.e., about 5.1±0.4). In another embodiment, the pharmaceutical composition of the invention has a pH of about 4.6 to about 5.4 (i.e., about 5.0±0.4). In another embodiment, the pharmaceutical composition of the invention has a pH of about 4.4 to about 5.2 (i.e., about 4.8±0.4). In another embodiment, the pharmaceutical composition of the invention has a pH of about 4.8. In another embodiment, the pharmaceutical composition of the invention has a pH of about 5.0. In another embodiment, the pharmaceutical composition of the invention has a pH of about 5.1 Also contemplated are pH amounts between any of these values, such as a pH of about 4.7, a pH of about 4.9, a pH of about 5.3, or a pH of about 5.5, etc.

In one embodiment, the pharmaceutical composition of the invention comprises about 5 mg/mL to about 200 mg/mL of enoblituzumab, about 5 mM to about 30 mM sodium acetate, about 50 mg/mL to about 130 mg/mL of sucrose, about 0.05 mg/mL to about 0.6 mg/mL of PS80, and water, wherein the composition has a pH of about 4.0 to about 6.0. In another embodiment, the pharmaceutical composition of the invention comprises about 5 mg/mL to about 200 mg/mL of enoblituzumab, about 8 mM to about 24 mM sodium acetate, about 72 mg/mL to about 108 mg/mL of sucrose, about 0.05 mg/mL to about 0.6 mg/mL of PS80, and water, wherein the composition has a pH of about 4.4 to about 5.6.

In one embodiment, the pharmaceutical composition of the invention comprises about 20 mg/mL to about 40 mg/mL of enoblituzumab, about 7.5 mM to about 15 mM sodium acetate, about 50 mg/mL to about 130 mg/mL of sucrose, about 0.05 mg/mL to about 0.6 mg/mL of PS80, and water, wherein the composition has a pH of about 4.0 to about 6.0. In another embodiment, the pharmaceutical composition of the invention comprises about 20 mg/mL to about 40 mg/mL of enoblituzumab about 8 mM to about 12 mM sodium acetate, about 72 mg/mL to about 108 mg/mL of sucrose, about 0.08 mg/mL to about 0.2 mg/mL of PS80, and water, wherein the composition has a pH of about 4.5 to about 5.5. In another embodiment, the pharmaceutical composition of the invention comprises about 20 mg/mL to about 30 mg/mL of enoblituzumab, about 9 mM to about 11 mM sodium acetate, about 76 mg/mL to about 104 mg/mL of sucrose, about 0.08 mg/mL to about 0.2 mg/mL of PS80, and water, wherein the composition has a pH of about 4.5 to about 5.5. In another embodiment, the pharmaceutical composition of the invention comprises about 25 mg/mL of enoblituzumab, about 10 mM sodium acetate, about 90 mg/mL of sucrose, about 0.1 mg/mL of PS80, and water, wherein the composition has a pH of about 4.6 to about 5.5. In another embodiment, the pharmaceutical composition of the invention comprises about 25 mg/mL of enoblituzumab, about 10 mM sodium acetate, about 90 mg/mL of sucrose, about 0.1 mg/mL of PS80, and water, wherein the composition has a pH of about 5.1. In another embodiment, the pharmaceutical composition of the invention comprises about 25 mg/mL of enoblituzumab, about 10 mM sodium acetate, about 90 mg/mL of sucrose, about 0.1 mg/mL of PS80, and water, wherein the composition has a pH of about 5.0.

In another embodiment, the pharmaceutical composition of the invention comprises about 60 mg/mL to about 200 mg/mL of enoblituzumab, about 16 mM to about 24 mM sodium acetate, about 72 mg/mL to about 108 mg/mL of sucrose, about 0.05 mg/mL to about 0.2 mg/mL of PS80, and water, wherein the composition has a pH of about 4.3 to about 5.3. In another embodiment, the pharmaceutical composition of the invention comprises about 60 mg/mL to about 200 mg/mL of enoblituzumab, about 18 mM to about 22 mM sodium acetate, about 76 mg/mL to about 104 mg/mL of sucrose, about 0.08 mg/mL to about 0.2 mg/mL of PS80, and water, wherein the composition has a pH of about 4.4 to about 5.2. In another embodiment, the pharmaceutical composition of the invention comprises about 90 mg/mL to about 200 mg/mL of enoblituzumab, about 20 mM sodium acetate, about 90 mg/mL of sucrose, about 0.1 mg/mL of PS80, and water, wherein the composition has a pH of about 4.4 to about 5.2. In another embodiment, the pharmaceutical composition of the invention comprises about 120 mg/mL of enoblituzumab, about 20 mM sodium acetate, about 90 mg/mL of sucrose, about 0.1 mg/mL of PS80, and water, wherein the composition has a pH of about 4.4 to about 5.2. In another embodiment, the pharmaceutical composition of the invention comprises about 120 mg/mL of enoblituzumab, about 20 mM sodium acetate, about 90 mg/mL of sucrose, about 0.1 mg/mL of PS80, and water, wherein the composition has a pH of about 4.8.

In one embodiment, the pharmaceutical composition of the invention comprises about 5 mg/mL to about 200 mg/mL of enoblituzumab, about 0.05 mg/mL to about 0.8 mg/mL of glacial acetic acid, about 0.5 mg/mL to about 2.0 mg/mL of sodium acetate trihydrate, about 50 mg/mL to about 130 mg/mL of sucrose, about 0.05 mg/mL to about 0.6 mg/mL of PS80, and water, wherein the composition has a pH of about 4.0 to about 6.0. In another embodiment, the pharmaceutical composition of the invention comprises about 5 mg/mL to about 200 mg/mL of enoblituzumab, about 0.1 mg/mL to about 0.65 mg/mL of glacial acetic acid, about 0.6 mg/mL to about 1.8 mg/mL of sodium acetate trihydrate, about 72 mg/mL to about 108 mg/mL of sucrose, about 0.05 mg/mL to about 0.6 mg/mL of PS80, and water, wherein the composition has a pH of about 4.4 to about 5.6.

In one embodiment, the pharmaceutical composition of the invention comprises about 20 mg/mL to about 30 mg/mL of enoblituzumab, about 0.1 mg/mL to about 0.35 mg/mL of glacial acetic acid, about 0.6 mg/mL to about 1.2 mg/mL of sodium acetate trihydrate, 72 mg/mL to about 108 mg/mL of sucrose, about 0.05 mg/mL to about 0.2 mg/mL of PS80, and water, wherein the composition has a pH of about 4.5 to about 5.5. In one embodiment, the pharmaceutical composition of the invention comprises about 22.5 mg/mL to about 27.5 mg/mL of enoblituzumab, about 0.1 mg/mL to about 0.35 mg/mL of glacial acetic acid, about 0.6 mg/mL to about 1.2 mg/mL of sodium acetate trihydrate, about 72 mg/mL to about 108 mg/mL of sucrose, about 0.05 mg/mL to about 0.2 mg/mL of PS80, and water, wherein the composition has a pH of about 4.5 to about 5.5. In one embodiment, the pharmaceutical composition of the invention comprises about 25 mg/mL of enoblituzumab, about 0.18 mg/mL glacial acetic acid, about 0.95 mg/mL sodium acetate trihydrate, about 90 mg/mL sucrose, about 0.1 mg/mL PS80, and water, wherein the composition has a pH of about 4.7 to about 5.5. In another embodiment, the pharmaceutical composition of the invention comprises about 25 mg/mL of enoblituzumab, about 0.18 mg/mL glacial acetic acid, about 0.95 mg/mL sodium acetate trihydrate, about 90 mg/mL of sucrose, about 0.1 mg/mL of PS80, and water, wherein the composition has a pH of about 5.1. In one embodiment, the pharmaceutical composition of the invention comprises about 25 mg/mL of enoblituzumab, about 0.27 mg/mL glacial acetic acid, about 0.74 mg/mL sodium acetate trihydrate, about 90 mg/mL of sucrose, about 0.1 mg/mL of PS80, and water, wherein the composition has a pH of about 4.6 to about 5.4. In another embodiment, the pharmaceutical composition of the invention comprises about 25 mg/mL of enoblituzumab, about 0.27 mg/mL glacial acetic acid, about 0.74 mg/mL sodium acetate trihydrate, about 90 mg/mL sucrose, about 0.1 mg/mL PS80, and water, wherein the composition has a pH of about 5.0.

In one embodiment, the pharmaceutical composition of the invention comprises about 60 mg/mL to about 200 mg/mL of enoblituzumab, about 0.4 mg/mL to about 0.65 mg/mL of glacial acetic acid, about 1.2 mg/mL to about 1.8 mg/mL of sodium acetate trihydrate, about 72 mg/mL to about 108 mg/mL of sucrose, about 0.05 mg/mL to about 0.2 mg/mL PS80, and water, wherein the composition has a pH of about 4.3 to about 5.3. In another embodiment, the pharmaceutical composition of the invention comprises about 90 mg/mL to about 200 mg/mL of enoblituzumab, about 0.4 mg/mL to about 0.65 mg/mL of glacial acetic acid, about 1.2 mg/mL to about 1.8 mg/mL of sodium acetate trihydrate, about 72 mg/mL to about 108 mg/mL of sucrose, about 0.05 mg/mL to about 0.2 mg/mL PS80, and water, wherein the composition has a pH of about 4.3 to about 5.3. In another embodiment, the pharmaceutical composition of the invention comprises about 120 mg/mL of enoblituzumab, about 0.52 mg/mL of glacial acetic acid, about 1.5 mg/mL of sodium acetate trihydrate, about 90 mg/mL of sucrose, about 0.1 mg/mL PS80, and water, wherein the composition has a pH of about 4.4 to about 5.2. In another embodiment, the pharmaceutical composition of the invention comprises about 120 mg/mL of enoblituzumab, about 0.52 mg/mL of glacial acetic acid, about 1.5 mg/mL of sodium acetate trihydrate, about 90 mg/mL of sucrose, about 0.1 mg/mL PS80, and water, wherein the composition has a pH of about 4.8.

In one embodiment, about 5 mL to about 20 mL of the pharmaceutical composition of the invention can comprise about 25 mg/mL of enoblituzumab, about 0.18 mg/mL glacial acetic acid, about 0.95 mg/mL sodium acetate trihydrate, about 90 mg/mL sucrose, about 0.1 mg/mL PS80, and water, wherein the composition has a pH of about 4.7 to about 5.5. In another embodiment, about 5 mL to about 20 mL of the pharmaceutical composition of the invention can comprise about 25 mg/mL of enoblituzumab, about 0.18 mg/mL glacial acetic acid, about 0.95 mg/mL sodium acetate trihydrate, about 90 mg/mL sucrose, about 0.1 mg/mL PS80, and water, wherein the composition has a pH of about 5.1.

In another embodiment about 5 mL to about 20 mL of the pharmaceutical composition of the invention can comprise about 25 mg/mL of enoblituzumab, about 0.27 mg/mL glacial acetic acid, about 0.74 mg/mL sodium acetate trihydrate, about 90 mg/mL sucrose, about 0.1 mg/mL PS80, and water, wherein the composition has a pH of about 4.6 to about 5.4. In another embodiment, about 5 mL to about 20 mL of the pharmaceutical composition of the invention can comprise about 25 mg/mL of enoblituzumab, about 0.27 mg/mL glacial acetic acid, about 0.74 mg/mL sodium acetate trihydrate, about 90 mg/mL sucrose, about 0.1 mg/mL PS80, and water, wherein the composition has a pH of about 5.0.

In one embodiment, about 5 mL to about 20 mL of the pharmaceutical composition of the invention can comprise about 120 mg/mL of enoblituzumab, about 20 mM sodium acetate, about 90 mg/mL sucrose, about 0.1 mg/mL PS80, and water, wherein the composition has a pH of about 4.4 to about 5.2. In another embodiment, about 5 mL to about 20 mL of the pharmaceutical composition of the invention can comprise about 120 mg/mL of enoblituzumab, about 20 mM sodium acetate, about 90 mg/mL sucrose, about 0.1 mg/mL PS80, and water, wherein the composition has a pH of about 4.8.

In another embodiment about 5 mL to about 20 mL of the pharmaceutical composition of the invention can comprise about 120 mg/mL of enoblituzumab, about 0.52 mg/mL glacial acetic acid, about 1.5 mg/mL sodium acetate trihydrate, about 90 mg/mL sucrose, about 0.1 mg/mL PS80, and water, wherein the composition has a pH of about 4.4 to about 5.2. In another embodiment, about 5 mL to about 20 mL of the pharmaceutical composition of the invention can comprise about 120 mg/mL of enoblituzumab, about 0.52 mg/mL glacial acetic acid, about 1.5 mg/mL sodium acetate trihydrate, about 90 mg/mL sucrose, about 0.1 mg/mL PS80, and water, wherein the composition has a pH of about 4.8.

In one embodiment, about 5 mL of the pharmaceutical composition of the invention comprises about 125 mg of enoblituzumab, about 10 mM sodium acetate, about 450 mg of sucrose, about 0.5 mg of PS80, and water, and wherein the composition has a pH of about 4.6 to about 5.5. In another embodiment, about 5 mL of the pharmaceutical composition of the invention comprises about 125 mg of enoblituzumab, about 10 mM sodium acetate, about 450 mg of sucrose, about 0.5 mg of PS80, and water, and wherein the composition has a pH of about 5.1. In another embodiment, about 5 mL of the pharmaceutical composition of the invention comprises about 125 mg of enoblituzumab, about 10 mM sodium acetate, about 450 mg of sucrose, about 0.5 mg of PS80, and water, and wherein the composition has a pH of about 5.0.

In one embodiment, about 5 mL of the pharmaceutical composition of the invention comprises about 125 mg of enoblituzumab, about 0.9 mg of glacial acetic acid, about 4.75 mg of sodium acetate trihydrate, about 450 mg of sucrose, about 0.5 mg of PS80, and water, and wherein the composition has a pH of about 4.7 to about 5.5. In another embodiment, about 5 mL of the pharmaceutical composition of the invention comprises about 125 mg of enoblituzumab about 0.9 mg of glacial acetic acid, about 4.75 mg of sodium acetate trihydrate, about 450 mg of sucrose, about 0.5 mg of PS80, and water, and wherein the composition has a pH of about 5.1. In another embodiment, about 5 mL of the pharmaceutical composition of the invention comprises about 125 mg of enoblituzumab about 0.9 mg of glacial acetic acid, about 4.75 mg of sodium acetate trihydrate, about 450 mg of sucrose, about 0.5 mg of PS80, and water, and wherein the composition has a pH of about 4.6 to about 5.4. In one embodiment, about 5 mL of the pharmaceutical composition of the invention comprises about 125 mg of enoblituzumab, about 1.35 mg of glacial acetic acid, about 3.7 mg of sodium acetate trihydrate, about 450 mg of sucrose, about 0.5 mg of PS80, and water, and wherein the composition has a pH of about 4.8.

In one embodiment, about 10 mL of the pharmaceutical composition of the invention comprises about 250 mg of enoblituzumab, about 10 mM sodium acetate, about 900 mg of sucrose, about 1 mg of PS80, and water, and wherein the composition has a pH of about 4.6 to about 5.5. In another embodiment, about 10 mL of the pharmaceutical composition of the invention comprises about 250 mg of enoblituzumab, about 10 mM sodium acetate, about 900 mg of sucrose, about 1 mg of PS80, and water, and wherein the composition has a pH of about 5.1. In another embodiment, about 10 mL of the pharmaceutical composition of the invention comprises about 250 mg of enoblituzumab, about 10 mM sodium acetate, about 900 mg of sucrose, about 1 mg of PS80, and water, and wherein the composition has a pH of about 5.0.

In one embodiment, about 10 mL of the pharmaceutical composition of the invention comprises about 250 mg of enoblituzumab, about 1.8 mg of glacial acetic acid, about 9.5 mg of sodium acetate trihydrate, about 900 mg of sucrose, about 1 mg of PS80, and water, and wherein the composition has a pH of about 4.7 to about 5.5. In another embodiment, about 10 mL of the pharmaceutical composition of the invention comprises about 250 mg of enoblituzumab about 1.8 mg of glacial acetic acid, about 9.5 mg of sodium acetate trihydrate, about 900 mg of sucrose, about 1 mg of PS80, and water, and wherein the composition has a pH of about 5.1. In another embodiment, about 10 mL of the pharmaceutical composition of the invention comprises about 250 mg of enoblituzumab about 1.8 mg of glacial acetic acid, about 9.5 mg of sodium acetate trihydrate, about 900 mg of sucrose, about 1 mg of PS80, and water, and wherein the composition has a pH of about 4.6 to about 5.4. In one embodiment, about 10 mL of the pharmaceutical composition of the invention comprises about 250 mg of enoblituzumab, about 2.7 mg of glacial acetic acid, about 7.4 mg of sodium acetate trihydrate, about 900 mg of sucrose, about 1 mg of PS80, and water, and wherein the composition has a pH of about 4.8.

In an alternative embodiment, about 17 mL of the pharmaceutical composition of the invention comprises about 425 mg of enoblituzumab, about 10 mM of sodium acetate trihydrate, about 1530 mg of sucrose, about 1.7 mg of PS80, and water, and wherein the composition has a pH of about 4.6 to about 5.5. In another alternative embodiment, about 17 mL of the pharmaceutical composition of the invention comprises about 425 mg of enoblituzumab, about 10 mM of sodium acetate trihydrate, about 1530 mg of sucrose, about 1.7 mg of PS80, and water, and wherein the composition has a pH of about 5.1. In another alternative embodiment, about 17 mL of the pharmaceutical composition of the invention comprises about 425 mg of enoblituzumab, about 10 mM of sodium acetate trihydrate, about 1530 mg of sucrose, about 1.7 mg of PS80, and water, and wherein the composition has a pH of about 5.0.

In another alternative embodiment, about 17 mL of the pharmaceutical composition of the invention comprises about 425 mg of enoblituzumab, about 3.06 mg of glacial acetic acid, about 16.15 mg of sodium acetate trihydrate, about 1530 mg of sucrose, about 1.7 mg of PS80, and water, and wherein the composition has a pH of about 4.7 to about 5.5. In another alternative embodiment, about 17 mL of the pharmaceutical composition of the invention comprises about 425 mg of enoblituzumab, about 3.06 mg of glacial acetic acid, about 16.15 mg of sodium acetate trihydrate, about 1530 mg of sucrose, about 1.7 mg of PS80, and water, and wherein the composition has a pH of about 5.1. In another alternative embodiment, about 17 mL of the pharmaceutical composition of the invention comprises about 425 mg of enoblituzumab, about 4.59 mg of glacial acetic acid, about 12.58 mg of sodium acetate trihydrate, about 1530 mg of sucrose, about 1.7 mg of PS80, and water, and wherein the composition has a pH of about 4.6 to about 5.4. In another alternative embodiment, about 17 mL of the pharmaceutical composition of the invention comprises about 425 mg of enoblituzumab, about 4.59 mg of glacial acetic acid, about 12.58 mg of sodium acetate trihydrate, about 1530 mg of sucrose, about 1.7 mg of PS80, and water, and wherein the composition has a pH of about 5.0.

In one embodiment, about 20 mL of the pharmaceutical composition of the invention comprises about 500 mg of enoblituzumab, about 10 mM sodium acetate, about 1800 mg of sucrose, about 2 mg of PS80, and water, and wherein the composition has a pH of about 4.6 to about 5.5. In another embodiment, about 20 mL of the pharmaceutical composition of the invention comprises about 500 mg of enoblituzumab, about 10 mM sodium acetate, about 1800 mg of sucrose, about 2 mg of PS80, and water, and wherein the composition has a pH of about 5.1. In another embodiment, about 20 mL of the pharmaceutical composition of the invention comprises about 500 mg of enoblituzumab, about 10 mM sodium acetate, about 1800 mg of sucrose, about 2 mg of PS80, and water, and wherein the composition has a pH of about 5.0.

In one embodiment, about 20 mL of the pharmaceutical composition of the invention comprises about 500 mg of enoblituzumab, about 3.6 mg of glacial acetic acid, about 19 mg of sodium acetate trihydrate, about 1800 mg of sucrose, about 2 mg of PS80, and water, and wherein the composition has a pH of about 4.7 to about 5.5. In another embodiment, about 20 mL of the pharmaceutical composition of the invention comprises about 500 mg of enoblituzumab about 3.6 mg of glacial acetic acid, about 19 mg of sodium acetate trihydrate, about 1800 mg of sucrose, about 2 mg of PS80, and water, and wherein the composition has a pH of about 5.1. In another embodiment, about 20 mL of the pharmaceutical composition of the invention comprises about 500 mg of enoblituzumab about 3.6 mg of glacial acetic acid, about 19 mg of sodium acetate trihydrate, about 1800 mg of sucrose, about 2 mg of PS80, and water, and wherein the composition has a pH of about 4.6 to about 5.4. In one embodiment, about 20 mL of the pharmaceutical composition of the invention comprises about 500 mg of enoblituzumab, about 5.4 mg of glacial acetic acid, about 14.8 mg of sodium acetate trihydrate, about 1800 mg of sucrose, about 2 mg of PS80, and water, and wherein the composition has a pH of about 4.8.

In one embodiment, about 10 mL of the pharmaceutical composition of the invention comprises about 1,200 mg of enoblituzumab about 20 mM sodium acetate, about 900 mg of sucrose, about 1 mg of PS80, and water, and wherein the composition has a pH of about 4.4 to about 5.2. In another embodiment, about 10 mL of the pharmaceutical composition of the invention comprises about 1,200 mg of enoblituzumab, about 20 mM sodium acetate, about 900 mg of sucrose, about 1 mg of PS80, and water, and wherein the composition has a pH of about 4.8. In one embodiment, about 10 mL of the pharmaceutical composition of the invention comprises about 1,200 mg of enoblituzumab about 5.2 mg glacial acetic acid, about 15 mg sodium acetate trihydrate, about 900 mg of sucrose, about 1 mg of PS80, and water, and wherein the composition has a pH of about 4.4 to about 5.2. In another embodiment, about 10 mL of the pharmaceutical composition of the invention comprises about 1,200 mg of enoblituzumab, about 5.2 mg glacial acetic acid, about 15 mg sodium acetate trihydrate, about 900 mg of sucrose, about 1 mg of PS80, and water, and wherein the composition has a pH of about 4.8.

In one embodiment, the pharmaceutical composition of the invention has an osmolality of about 200 to about 400 mOsm/kg H₂O In another embodiment, the pharmaceutical composition of the invention has an osmolality of about 225 to about 375 mOsm/kg. In another embodiment, the pharmaceutical composition of the invention has an osmolality of about 250 to about 360 mOsm/kg. In another embodiment, the pharmaceutical composition of the invention has an osmolality of about 260 to about 340 mOsm/kg H₂O.

In certain embodiments, the pharmaceutical composition of the disclosure is sterile. In one embodiment, the pharmaceutical composition of the disclosure is nonpyrogenic. The disclosure further provides an embodiment of such pharmaceutical compositions, sealed packages or kits wherein the water is sterile, nonpyrogenic, distilled water. In another embodiment, the water in the sealed packages, kits or pharmaceutical composition of the disclosure is Water for Injection, USP, or the equivalent.

In one embodiment, the pharmaceutical composition of the invention is stable for at least about 3 months at about 25° C. In another embodiment, the pharmaceutical composition of the invention maintains monomeric purity of enoblituzumab for at least about 3 months at about 25° C. In another embodiment, the loss of monomeric purity of enoblituzumab in the pharmaceutical composition is about 5% or less over about 3 months at about 25° C. In another embodiment, the loss of monomeric purity of enoblituzumab in the pharmaceutical composition is about 3% or less over about 3 months at about 25° C. In another embodiment, the pharmaceutical composition of the invention maintains the charge heterogeneity profile of enoblituzumab for at least about 3 months at about 25° C. In another embodiment, the decrease in the main charge peak (MCP) of enoblituzumab in the pharmaceutical composition is about 20% or less over about 3 months at about 25° C. In another embodiment, the increase in the AV of enoblituzumab in the pharmaceutical composition is about 20% or less over about 3 months at about 25° C.

In one embodiment, the pharmaceutical composition of the invention is stable for at least about 6 months at about 25° C. In another embodiment, the pharmaceutical composition of the invention maintains monomeric purity of enoblituzumab for at least about 6 months at about 25° C. In another embodiment, the loss of monomeric purity of enoblituzumab in the pharmaceutical composition is about 6% or less over about 6 months at 25° C. In another embodiment, the loss of monomeric purity of enoblituzumab in the pharmaceutical composition is about 5% or less over about 6 months at about 25° C. In another embodiment, the loss of monomeric purity of enoblituzumab in the pharmaceutical composition is about 3% or less over about 6 months at about 25° C. In another embodiment, the pharmaceutical composition of the invention maintains the charge heterogeneity profile of enoblituzumab for at least about 6 months at about 25° C. In another embodiment, the decrease in the MCP of enoblituzumab in the pharmaceutical composition is about 20% or less over about 6 months at about 25° C. In another embodiment, the increase in the AV of enoblituzumab in the pharmaceutical composition is about 20% or less over about 6 months at about 25° C.

In one embodiment, pharmaceutical composition of the invention is stable for at least about 18 months at about 2° C. to about 8° C. In another embodiment the pharmaceutical composition of the invention maintains monomeric purity of enoblituzumab for at least about 18 months at about 2° C. to about 8° C. In another embodiment, the loss of monomeric purity of enoblituzumab in the pharmaceutical composition is about 5% or less over about 18 months at about 2° C. to about 8° C. In another embodiment, the loss of monomeric purity of enoblituzumab in the pharmaceutical composition is about 4% or less over about 18 months at about 2° C. to about 8° C. In another embodiment, the loss of monomeric purity of enoblituzumab in the pharmaceutical composition is about 3% or less over about 18 months at about 2° C. to about 8° C. In another embodiment the pharmaceutical composition of the invention maintains the charge heterogeneity profile of enoblituzumab for at least about 18 months at about 2° C. to about 8° C. In another embodiment, the decrease in the MCP of enoblituzumab in the pharmaceutical composition is about 10% or less over about 18 months at about 2° C. to about 8° C. In another embodiment, the decrease in the MCP of enoblituzumab in the pharmaceutical composition is about 9% or less over about 18 months at about 2° C. to about 8° C. In another embodiment, the decrease in the MCP of enoblituzumab in a pharmaceutical composition or in a dosing solution is about 7% or less over about 18 months at about 2° C. to about 8° C. In another embodiment, the decrease in the MCP of enoblituzumab in a pharmaceutical composition or in a dosing solution is about 5% or less over about 18 months at about 2° C. to about 8° C. In another embodiment, the increase in the AV of enoblituzumab in the pharmaceutical composition is about 5% or less over about 18 months at about 2° C. to about 8° C. In another embodiment, the increase in the AV of enoblituzumab in the pharmaceutical composition is about 4% or less over about 18 months at about 2° C. to about 8° C. In another embodiment, the increase in the AV of enoblituzumab in the pharmaceutical composition is about 3% or less over about 18 months at about 2° C. to about 8° C.

In one embodiment, the pharmaceutical composition of the invention is stable for at least about 24 months at about 2° C. to about 8° C. In another embodiment the pharmaceutical composition of the invention maintains monomeric purity of enoblituzumab for at least about 24 months at about 2° C. to about 8° C. In another embodiment, the loss of monomeric purity of enoblituzumab in the pharmaceutical composition is about 5% or less over about 24 months at about 2° C. to about 8° C. In another embodiment, the loss of monomeric purity of enoblituzumab in the pharmaceutical composition is about 4% or less over about 24 months at about 2° C. to about 8° C. In another embodiment, the loss of monomeric purity of enoblituzumab in the pharmaceutical composition is about 3% or less over about 24 months at about 2° C. to about 8° C. In another embodiment the pharmaceutical composition of the invention maintains the charge heterogeneity profile of enoblituzumab for at least about 24 months at about 2° C. to about 8° C. In another embodiment, the decrease in the MCP of enoblituzumab in the pharmaceutical composition is about 10% or less over about 24 months at about 2° C. to about 8° C. In another embodiment, the decrease in the MCP of enoblituzumab in the pharmaceutical composition is about 9% or less over about 24 months at about 2° C. to about 8° C. In another embodiment, the decrease in the MCP of enoblituzumab in the pharmaceutical composition is about 7% or less over about 24 months at about 2° C. to about 8° C. In another embodiment, the decrease in the MCP of enoblituzumab in the pharmaceutical composition is about 5% or less over about 24 months at about 2° C. to about 8° C. In another embodiment, the increase in the AV of enoblituzumab in the pharmaceutical composition is about 5% or less over about 24 months at about 2° C. to about 8° C. In another embodiment, the increase in the AV of enoblituzumab in the pharmaceutical composition is about 4% or less over about 24 months at about 2° C. to about 8° C. In another embodiment, the increase in the AV of enoblituzumab in the pharmaceutical composition is about 3% or less over about 24 months at about 2° C. to about 8° C.

In one embodiment, the pharmaceutical composition of the invention is stable for about 36 months at about 2° C. to about 8° C. In another embodiment the pharmaceutical composition of the invention maintains monomeric purity of enoblituzumab for about 36 months at about 2° C. to about 8° C. In another embodiment, the loss of monomeric purity of enoblituzumab in the pharmaceutical composition is about 5% or less over about 36 months at about 2° C. to about 8° C. In another embodiment, the loss of monomeric purity of enoblituzumab in the pharmaceutical composition is about 4% or less over about 36 months at about 2° C. to about 8° C. In another embodiment, the loss of monomeric purity of enoblituzumab in the pharmaceutical composition is about 3% or less over about 36 months at about 2° C. to about 8° C. In another embodiment the pharmaceutical composition of the invention maintains the charge heterogeneity profile of enoblituzumab for at least about 36 months at about 2° C. to about 8° C. In another embodiment, the decrease in the MP of enoblituzumab in the pharmaceutical composition is about 10% or less over about 36 months at about 2° C. to about 8° C. In another embodiment, the decrease in the MCP of enoblituzumab in the pharmaceutical composition is about 9% or less over about 36 months at about 2° C. to about 8° C. In another embodiment, the decrease in the MCP of enoblituzumab in the pharmaceutical composition is about 7% or less over about 36 months at about 2° C. to about 8° C. In another embodiment, the decrease in the MCP of enoblituzumab in the pharmaceutical composition is about 5% or less over about 36 months at about 2° C. to about 8° C. In another embodiment, the increase in the AV of enoblituzumab in the pharmaceutical composition is about 5% or less over about 36 months at about 2° C. to about 8° C. In another embodiment, the increase in the AV of enoblituzumab in the pharmaceutical composition is about 4% or less over about 36 months at about 2° C. to about 8° C. In another embodiment, the increase in the AV of enoblituzumab in the pharmaceutical composition is about 3% or less over about 36 months at about 2° C. to about 8° C.

In one embodiment, pharmaceutical composition of the invention is stable for about 48 months at about 2° C. to about 8° C. In another embodiment the pharmaceutical composition of the invention maintains monomeric purity of enoblituzumab for about 48 months at about 2° C. to about 8° C. In another embodiment, the loss of monomeric purity of enoblituzumab in the pharmaceutical composition is about 5% or less over about 48 months at about 2° C. to about 8° C. In another embodiment, the loss of monomeric purity of enoblituzumab in the pharmaceutical composition is about 4% or less over about 48 months at about 2° C. to about 8° C. In another embodiment, the loss of monomeric purity of enoblituzumab in the pharmaceutical composition is about 3% or less over about 48 months at about 2° C. to about 8° C. In another embodiment, the decrease in the MP of enoblituzumab in the pharmaceutical composition is about 10% or less over about 48 months at about 2° C. to about 8° C. In another embodiment, the decrease in the MCP of enoblituzumab in the pharmaceutical composition is about 9% or less over about 48 months at about 2° C. to about 8° C. In another embodiment, the decrease in the MCP of enoblituzumab in the pharmaceutical composition is about 7% or less over about 48 months at about 2° C. to about 8° C. In another embodiment, the decrease in the MCP of enoblituzumab in the pharmaceutical composition is about 5% or less over about 48 months at about 2° C. to about 8° C. In another embodiment, the increase in the AV of enoblituzumab in the pharmaceutical composition is about 5% or less over about 48 months at about 2° C. to about 8° C. In another embodiment, the increase in the AV of enoblituzumab in the pharmaceutical composition is about 4% or less over about 48 months at about 2° C. to about 8° C. In another embodiment, the increase in the AV of enoblituzumab in the pharmaceutical composition is about 3% or less over about 48 months at about 2° C. to about 8° C.

### III. Containers and Kits

The invention also provides containers comprising a pharmaceutical composition of the invention. The invention further provides pharmaceutical packs or kits comprising one or more containers containing a pharmaceutical composition of the invention. In one embodiment, such container is a vial (e.g., a single-dose vial). In one embodiment, such pharmaceutical pack or kit of the invention contains a vial (e.g., single-dose vial). In another embodiment, such pharmaceutical pack or kit contains more than one vial.

In one embodiment, such container (e.g., vials) contain about 5 mL to about 20 mL of a pharmaceutical composition of the invention. In one embodiment, such container (e.g., vials) contain about 5 mL of a pharmaceutical composition of the invention comprising about 125 mg of enoblituzumab such that the concentration of enoblituzumab is about 25 mg/mL per container. In another embodiment, such containers (e.g., vials) contain about 10 mL of a pharmaceutical composition of the invention comprising about 250 mg of enoblituzumab such that the concentration of enoblituzumab is about 25 mg/mL per container. In another embodiment, such containers (e.g., vials) contain about 10 mL of a pharmaceutical composition of the invention comprising about 1,200 mg of enoblituzumab such that the concentration of enoblituzumab is about 120 mg/mL per container. In another embodiment, such containers (e.g., vials) contain about 17 mL of a pharmaceutical composition of the invention comprising about 425 mg of enoblituzumab such that the concentration of enoblituzumab is about 25 mg/mL per container. In another embodiment, such containers (e.g., vials) contain about 20 mL of a pharmaceutical composition of the invention comprising about 500 mg of enoblituzumab such that the concentration of enoblituzumab is about 25 mg/mL per container. It is appreciated that such containers (e.g., vials) may comprise an overfill volume of such pharmaceutical composition of the invention to ensure sufficient volume for withdrawal of up to about 5 mL (125 mg), up to about 10 mL (250 mg or 1,200 mg), up to about 17 mL (425 mg), and up to about 20 mL (500 mg) of enoblituzumab for dose delivery.

Additionally, one or more other prophylactic or therapeutic agents useful for the treatment of a disease can also be included in the pharmaceutical pack or kit of the invention. Optionally associated with such container(s) can be a notice in the form prescribed by a governmental agency regulating the manufacture, use or sale of pharmaceuticals or biological products, which notice reflects approval by the agency of manufacture, use or sale for human administration. Optionally associated with such container(s) is a product label describing the indication(s) and/or instructions for preparation and administration of a dosing solution comprising the enoblituzumab composition.

The present invention provides kits that comprise a pharmaceutical composition of the invention (i.e., an enoblituzumab composition) that can be used in the above methods. In such kits, the pharmaceutical composition of the invention (i.e., an enoblituzumab composition) is generally packaged in hermetically sealed containers, such as ampoules, vials, sachets, or other suitable containers, for example, that typically indicate the quantity of the components contained therein. The container may be formed of any pharmaceutically acceptable material, such as glass, resin, plastic, or other suitable material. In one embodiment, the container is borosilicate glass vial. In another embodiment, the container is single-dose 5 mL USP Type I borosilicate glass vial. In another embodiment, the 5 mL container, contains about 125 mg enoblituzumab in a 5 mL volume. In one embodiment, the container is borosilicate glass vial. In another embodiment, the container is single-dose 10 mL USP Type I borosilicate glass vial. In another embodiment, the 10 mL container, contains about 250 mg enoblituzumab in a 10 mL volume. In another embodiment, the container is single-dose 20 mL USP Type I borosilicate glass vial. In another embodiment, the 20 mL container, contains about 425 mg enoblituzumab in a 17 mL volume. In another embodiment, the 20 mL container contains about 500 mg enoblituzumab in a 20 mL volume. In one embodiment, the container is aseptically filled. In one embodiment, the pharmaceutical compositions of the invention comprising such kits are supplied as a liquid solutions. Such liquid solutions are can be stored at between about 2° C. and about 8° C. in the original containers until ready to be administered. However, such solutions may be stored at room temperature (˜25° C.) for short periods of time. In one embodiment, such pharmaceutical compositions of the invention have a shelf-life of at least about 18 months at about 2° C. to about 8° C. In one embodiment, such pharmaceutical compositions of the invention have a shelf-life of at least about 24 months at about 2° C. to about 8° C. In one embodiment, such pharmaceutical compositions have a shelf-life of about 36 months at about 2° C. to about 8° C. In one embodiment, such pharmaceutical compositions have a shelf-life of at least about 48 months at about 2° C. to about 8° C. In other embodiments, such pharmaceutical compositions of the invention have a shelf-life of at least about 3 months at 25° C. In other embodiments, such pharmaceutical compositions of the invention have a shelf-life of at least about 6 months at 25° C. The kit can further comprise one or more other prophylactic and/or therapeutic agents, for example in a prophylactically effective amount or therapeutically effective amount, useful for the treatment of cancer, in one or more containers; and/or the kit can further comprise one or more antibodies, for example cytotoxic antibodies, that bind one or more cancer antigens associated with cancer. In certain embodiments, the other prophylactic or therapeutic agent is a chemotherapeutic. In other embodiments, the prophylactic or therapeutic agent is a biological or hormonal therapeutic.

Accordingly, the present invention provides kits comprising:
a) a container comprising a pharmaceutical composition as described herein; and
b) optionally, instructions for administration of the pharmaceutical composition to a subject in need thereof.

In one embodiment, such container can comprise a pharmaceutical composition comprising about 5 mg/mL to about 200 mg/mL of enoblituzumab, about 5 mM to about 30 mM sodium acetate, about 50 mg/mL to about 130 mg/mL of sucrose, about 0.05 mg/mL to about 0.6 mg/mL of PS80, and water, wherein the composition has a pH of about 4.0 to about 6.0. In one embodiment, such container can comprise a pharmaceutical composition comprising about 5 mg/mL to about 200 mg/mL of enoblituzumab, about 8 mM to about 24 mM sodium acetate, about 72 mg/mL to about 108 mg/mL of sucrose, about 0.05 mg/mL to about 0.6 mg/mL of PS80, and water, wherein the composition has a pH of about 4.4 to about 5.6.

In one embodiment, such container can comprise a pharmaceutical composition, the composition comprising about 20 mg/mL to about 40 mg/mL of enoblituzumab, about 7.5 mM to about 15 mM sodium acetate, about 50 mg/mL to about 130 mg/mL of sucrose, about 0.05 mg/mL to about 0.6 mg/mL of PS80, and water, wherein the composition has a pH of about 4.0 to about 6.0. In one embodiment, such container can comprise a pharmaceutical composition, the composition comprising about 20 mg/mL to about 40 mg/mL of enoblituzumab, about 8 mM to about 12 mM sodium acetate, about 72 mg/mL to about 108 mg/mL of sucrose, about 0.05 mg/mL to about 0.2 mg/mL of PS80, and water, wherein the composition has a pH of about 4.5 to about 5.5. In one embodiment, such container can comprise a pharmaceutical composition, the composition comprising about 20 mg/mL to about 30 mg/mL of enoblituzumab, about 9 mM to about 11 mM sodium acetate, about 72 mg/mL to about 104 mg/mL of sucrose, about 0.08 mg/mL to about 0.2 mg/mL of PS80, and water, wherein the composition has a pH of about 4.5 to about 5.5. In one embodiment, such container can comprise a pharmaceutical composition, the composition comprising about 25 mg/mL of enoblituzumab, about 10 mM sodium acetate, about 90 mg/mL of sucrose, about 0.1 mg/mL of PS80, and water, wherein the composition has a pH of about 4.6 to about 5.5. In one embodiment, such container can comprise a pharmaceutical composition, the composition comprising about 25 mg/mL of enoblituzumab, about 10 mM sodium acetate, 90 mg/mL of sucrose, about 0.1 mg/mL of PS80, and water, wherein the composition has a pH of about 5.1. In another embodiment, such container can comprise a pharmaceutical composition, the composition comprising about 25 mg/mL of enoblituzumab, about 10 mM sodium acetate, about 90 mg/mL of sucrose, about 0.1 mg/mL of PS80, and water, wherein the composition has a pH of about 5.0. In one embodiment, such container can comprise a pharmaceutical composition, the composition comprising about 60 mg/mL to about 130 mg/mL of enoblituzumab, about 16 mM to about 24 mM sodium acetate, about 72 mg/mL to about 108 mg/mL of sucrose, about 0.05 mg/mL to about 0.6 mg/mL of PS80, and water, wherein the composition has a pH of about 4.3 to about 5.3.

In one embodiment, such container can comprise a pharmaceutical composition, the composition comprising about 60 mg/mL to about 200 mg/mL of enoblituzumab, about 18 mM to about 22 mM sodium acetate, about 72 mg/mL to about 104 mg/mL of sucrose, about 0.08 mg/mL to about 0.2 mg/mL of PS80, and water, wherein the composition has a pH of about 4.4 to about 5.2. In one embodiment, such container can comprise a pharmaceutical composition, the composition comprising about 120 mg/mL of enoblituzumab, about 20 mM sodium acetate, about 90 mg/mL of sucrose, about 0.1 mg/mL of PS80, and water, wherein the composition has a pH of about 4.4 to about 5.2. In one embodiment, such container can comprise a pharmaceutical composition, the composition comprising about 120 mg/mL of enoblituzumab, about 20 mM sodium acetate, about 90 mg/mL of sucrose, about 0.1 mg/mL of PS80, and water, wherein the composition has a pH of about 4.8. In one embodiment, such container can comprise a pharmaceutical composition, the composition comprising about 5 mg/mL to about 200 mg/mL of enoblituzumab, about 0.05 mg/mL to about 0.8 mg/mL of glacial acetic acid, about 0.5 mg/mL to about 2.0 mg/mL of sodium acetate trihydrate, about 50 mg/mL to about 130 mg/mL of sucrose, about 0.05 mg/mL to about 0.6 mg/mL of PS80, and water, wherein the composition has a pH of about 4.0 to about 6.0.

In one embodiment, such container can comprise a pharmaceutical composition, the composition comprising about 5 mg/mL to about 200 mg/mL of enoblituzumab, about 0.1 mg/mL to about 0.65 mg/mL of glacial acetic acid, about 0.6 mg/mL to about 1.8 mg/mL of sodium acetate trihydrate, about 72 mg/mL to about 108 mg/mL of sucrose, about 0.05 mg/mL to about 0.6 mg/mL of PS80, and water, wherein the composition has a pH of about 4.4 to about 5.6. In one embodiment, such container can comprise a pharmaceutical composition, the composition comprising about 20 mg/mL to about 30 mg/mL of enoblituzumab, about 0.1 mg/mL to about 0.35 mg/mL of glacial acetic acid, about 0.6 mg/mL to about 1.2 mg/mL of sodium acetate trihydrate, about 72 mg/mL to about 108 mg/mL of sucrose, about 0.05 mg/mL to about 0.2 mg/mL of PS80, and water, wherein the composition has a pH of about 4.5 to about 5.5.

In another embodiment, such container can comprise a pharmaceutical composition, the composition comprising about 22.5 mg/mL to about 27.5 mg/mL of enoblituzumab, about 0.1 mg/mL to about 0.35 mg/mL of glacial acetic acid, about 0.6 mg/mL to about 1.2 mg/mL of sodium acetate trihydrate, about 72 mg/mL to about 108 mg/mL of sucrose, about 0.05 mg/mL to about 0.2 mg/mL of PS80, and water, wherein the composition has a pH of about 4.5 to about 5.5. In one embodiment, such container can comprise about 25 mg/mL of enoblituzumab, about 0.18 mg/mL glacial acetic acid, about 0.95 mg/mL sodium acetate trihydrate, about 90 mg/mL sucrose, about 0.1 mg/mL PS80, and water, wherein the composition has a pH of about 5.4 to about 5.7. In another embodiment, such container can comprise about 25 mg/mL of enoblituzumab, about 0.18 mg/mL glacial acetic acid, about 0.95 mg/mL sodium acetate trihydrate, about 90 mg/mL sucrose, about 0.1 mg/mL PS80, and water, wherein the composition has a pH of about 5.1. In one embodiment, such container can comprise about 25 mg/mL of enoblituzumab, about 0.27 mg/mL glacial acetic acid, about 0.74 mg/mL sodium acetate trihydrate, about 90 mg/mL sucrose, about 0.1 mg/mL PS80, and water, wherein the composition has a pH of about 4.6 to about 5.4. In another embodiment, such container can comprise about 25 mg/mL of enoblituzumab, about 0.27 mg/mL glacial acetic acid, about 0.74 mg/mL sodium acetate trihydrate, about 90 mg/mL sucrose, about 0.1 mg/mL PS80, and water, wherein the composition has a pH of about 5.0. In one embodiment, such container can comprise about 60 mg/mL to about 130 mg/mL of enoblituzumab, about 0.4 mg/mL to about 0.65 mg/mL of glacial acetic acid, 1.2 mg/mL to about 1.8 mg/mL of sodium acetate trihydrate, about 72 mg/mL to about 108 mg/mL of sucrose, about 0.05 mg/mL to about 0.2 mg/mL PS80, and water, wherein the composition has a pH of about 4.3 to about 5.3. In one embodiment, such container can comprise about 90 mg/mL to about 130 mg/mL of enoblituzumab, about 0.4 mg/mL to about 0.65 mg/mL of glacial acetic acid, about 1.2 mg/mL to about 1.8 mg/mL of sodium acetate trihydrate, about 72 mg/mL to about 108 mg/mL of sucrose, about 0.05 mg/mL to about 0.2 mg/mL PS80, and water, wherein the composition has a pH of about 4.3 to about 5.3.

In one embodiment, such container can comprise about 120 mg/mL of enoblituzumab, about 0.52 mg/mL of glacial acetic acid, about 1.5 mg/mL of sodium acetate trihydrate, about 90 mg/mL of sucrose, about 0.1 mg/mL PS80, and water, wherein the composition has a pH of about 4.4 to about 5.2. In another embodiment, such container can comprise about 120 mg/mL of enoblituzumab, about 0.52 of glacial acetic acid, about 1.5 mg/mL of sodium acetate trihydrate, about 90 mg/mL of sucrose, about 0.1 mg/mL PS80, and water, wherein the composition has a pH of about 4.8.

The water in such compositions, containers, sealed packages, and kits of the invention can be sterile, nonpyrogenic, distilled water, and can be Water for Injection, USP, or the equivalent.

In one embodiment, pharmaceutical kits of the invention or sealed packages of the invention can include instructional material. The included instructional material of the pharmaceutical kits of the invention or sealed packages of the invention can instruct that the provided pharmaceutical composition is to be administered in combination with an additional agent which may be provided in the same pharmaceutical kit or sealed package or in a separate pharmaceutical kit or separate sealed package. Such instructional material can instruct that the provided pharmaceutical composition is to be administered once about every 2 weeks, once about every 3 weeks, once about every 4 weeks, or more or less often at regular or irregular intervals. Such instructional material can instruct that a provided container of pharmaceutical composition comprises about 25 mg/mL (e.g., 125 mg/5 mL, 250 mg/10 mL, 425 mg/17 mL, or 500 mg/20 mL) or about 120 mg/mL (e.g., 1,200 mg/10 mL) of enoblituzumab. Such instructional material can instruct that the provided pharmaceutical composition is to be administered at a weight-based treatment dose of about 6 mg/kg, about 10 mg/kg, or about 15 mg/kg. Such instructional material may instruct that the provided pharmaceutical composition is to be diluted (e.g., in 0.9% sodium chloride or D5W) prior to administration. The included instructional material of the pharmaceutical kits of the invention or the sealed packages of the invention can combine any set of such information (e.g., it may instruct that an enoblituzumab pharmaceutical composition is to be diluted in 0.9% sodium chloride or D5W and administered at a weight-based treatment dose of about 6 mg/kg, about 10 mg/kg, or about 15 mg/kg and that such dose is to be administered once about every 2 weeks; once about every 3 weeks; about every 4 weeks, or more or less often at regular or irregular intervals). Such instructional material can instruct regarding the mode of administration of the included pharmaceutical composition, for example that it is to be administered by intravenous (IV) infusion. The included instructional material of the pharmaceutical kits of the invention or the sealed packages of the invention can instruct regarding the duration or timing of such administration, for example that the included pharmaceutical composition is composition is to be administered by intravenous (IV) infusion over about 30 minutes, over about 60 minutes, or over 120 minutes, or for longer or shorter durations. Up to 10 additional minutes of infusion time (i.e., up to a total of 130 minutes) are permitted to allow for flushing the line.

In one embodiment, the instructional material of the pharmaceutical kits of the invention instructs that the pharmaceutical composition is diluted in 0.9% sodium chloride to obtain a dosing solution. In another embodiment, the instructional material of the pharmaceutical kits of the invention instructs that the provided pharmaceutical composition is diluted in D5W to obtain a dosing solution.

In one embodiment, the instructional material of the sealed packages of the invention instructs that the pharmaceutical composition is diluted in 0.9% sodium chloride to obtain a dosing solution. In another embodiment, the instructional material of the sealed packages of the invention instructs that the pharmaceutical composition is diluted in D5W to obtain a dosing solution.

In one embodiment, the instructional material of the pharmaceutical kits of the invention provides a method of administering a pharmaceutical composition of the invention to a subject in need thereof, wherein in the method comprises:
a) diluting the pharmaceutical composition in a container in 0.9% sodium chloride to obtain a dosing solution;
b) inverting the container to mix the diluted solution; and
c) attaching the container containing the dosing solution to a device for administration to the subject.

In one embodiment, the instructional material of the sealed packages of the invention provides a method of administering a pharmaceutical composition of the invention to a subject in need thereof, wherein in the method comprises:
a) diluting the pharmaceutical composition in a container in 0.9% sodium chloride to obtain a dosing solution;
b) inverting the container to mix the diluted solution; and
c) attaching the container containing the dosing solution to a device for administration to the subject.

In one embodiment, the administration of the dosing solution of the invention is by intravenous (IV) infusion over a period of about 30 minutes to about 120 minutes, about 30 minutes, about 60 minutes, or about 120 minutes.

In one embodiment, the instructional material of the pharmaceutical kits of the invention provides a method of administering a pharmaceutical composition of the invention to a subject in need thereof, wherein in the method comprises:
a) diluting the pharmaceutical composition in a container in D5W to obtain a dosing solution;
b) inverting the container to mix the diluted solution; and
c) attaching the container containing the dosing solution to a device for administration to the subject.

In one embodiment, the instructional material of the sealed packages of the invention provides a method of administering a pharmaceutical composition of the invention to a subject in need thereof, wherein in the method comprises:
a) diluting the pharmaceutical composition in a container in D5W to obtain a dosing solution;
b) inverting the container to mix the diluted solution; and
c) attaching the container containing the dosing solution to a device for administration to the subject.

In one embodiment, the container is an IV bag containing 0.9% sodium chloride. In another embodiment, the container is an IV bag containing D5W. In another embodiment, the container is a syringe containing 0.9% sodium chloride. In another embodiment, the container is a syringe containing D5W.

In one embodiment, the pharmaceutical composition of the invention is diluted to obtain a weight-based treatment dose of about 6 mg/kg to about 15 mg/kg of enoblituzumab in the dosing solution. In another embodiment, the pharmaceutical composition of the invention is diluted to obtain a weight-based treatment dose of about 6 mg/kg of enoblituzumab in the dosing solution. In another embodiment, the pharmaceutical composition of the invention is diluted to obtain a weight-based treatment dose of about 10 mg/kg of enoblituzumab in the dosing solution. In another embodiment, the pharmaceutical composition of the invention is diluted to obtain a weight-based treatment dose of about 15 mg/kg of enoblituzumab in the dosing solution.

The included instructional material of the pharmaceutical kits of the invention or the sealed packages of the invention may instruct regarding the appropriate or desired use of the included pharmaceutical composition, for example instructing that the provided pharmaceutical composition is to be administered for the treatment of cancer, for example in a prophylactically effective amount or therapeutically effective amount. In one embodiment such cancer is selected from the group consisting of: adrenal gland cancer, AIDS-associated cancer, alveolar soft part sarcoma, anal cancer (including squamous cell carcinoma of the anal canal (SCAC)), bladder cancer, bone cancer, brain and spinal cord cancer, breast cancer (including, HER2⁺ breast cancer or Triple-Negative Breast Cancer (TNBC)), carotid body tumor, cervical cancer (including, HPV-related cervical cancer), chondrosarcoma, chordoma, chromophobe renal cell carcinoma, clear cell carcinoma, colon cancer, colorectal cancer, desmoplastic small round cell tumor, ependymoma, endometrial cancer (including, unselected endometrial cancer, MSI-high endometrial cancer, dMMR endometrial cancer, and/or POLE exonuclease domain mutation positive endometrial cancer), Ewing's sarcoma, extraskeletal myxoid chondrosarcoma, gallbladder or bile duct cancer (including, cholangiocarcinoma bile duct cancer), gastric cancer, gastroesophageal junction (GEJ) cancer, gestational trophoblastic disease, germ cell tumor, glioblastoma, head and neck cancer (including, squamous cell carcinoma of head and neck (SCCHN)), a hematological malignancy, a hepatocellular carcinoma, islet cell tumor, Kaposi's Sarcoma, kidney cancer, leukemia (including, acute myeloid leukemia), liposarcoma/malignant lipomatous tumor, liver cancer (including, hepatocellular carcinoma liver cancer (HCC)), lymphoma (including, diffuse large B-cell lymphoma (DLBCL), non-Hodgkin's lymphoma (NHL)), lung cancer (including, small cell lung cancer (SCLC), non-small cell lung cancer (NSCLC)), medulloblastoma, melanoma (including, uveal melanoma), meningioma, Merkel cell carcinoma, mesothelioma (including, mesothelial pharyngeal cancer), multiple endocrine neoplasia, multiple myeloma, myelodysplastic syndrome, neuroblastoma, neuroendocrine tumors, ovarian cancer, pancreatic cancer, papillary thyroid carcinoma, parathyroid tumor, pediatric cancer, peripheral nerve sheath tumor, pharyngeal cancer, pheochromocytoma, pituitary tumor, prostate cancer (including, metastatic castration resistant prostate cancer (mCRPC)), posterious uveal melanoma, renal metastatic cancer, rhabdoid tumor, rhabdomyosarcoma, sarcoma, skin cancer, a small round blue cell tumor of childhood (including neuroblastoma and rhabdomyosarcoma), soft-tissue sarcoma, squamous cell cancer, stomach cancer, synovial sarcoma, testicular cancer, thymic carcinoma, thymoma, thyroid cancer, urothelial cancer, and uterine cancer.

The included instructional material of the pharmaceutical kits of the invention or sealed packages of the invention may instruct that such pharmaceutical composition is to be administered for a cancer selected from the group consisting of: anal cancer, bladder cancer, breast cancer, bile duct cancer, cervical cancer, colorectal cancer, endometrial cancer, gastric cancer, GEJ cancer, head and neck cancer, liver cancer, lung cancer, lymphoma, ovarian cancer, prostate cancer, skin cancer, and urothelial cancer.

In one embodiment, included instructional material of the pharmaceutical kits of the invention or the sealed packages of the invention instructs that such pharmaceutical composition is to be administered for the treatment of anal cancer. In another embodiment, the anal cancer is SCAC).

In one embodiment, included instructional material of the pharmaceutical kits of the invention or the sealed packages of the invention instructs that such pharmaceutical composition is to be administered for the treatment of lung cancer. In another embodiment, the lung cancer is NSCLC.

In one embodiment, included instructional material of the pharmaceutical kits of the invention or the sealed packages of the invention instructs that such pharmaceutical composition is to be administered for the treatment of breast cancer. In another embodiment, the breast cancer is TNBC.

In one embodiment, included instructional material of the pharmaceutical kits of the invention or the sealed packages of the invention instructs that such pharmaceutical composition is to be administered for the treatment of skin cancer. In another embodiment, the skin cancer is melanoma. In another embodiment, the skin cancer is Merkel cell carcinoma.

In one embodiment, included instructional material of the pharmaceutical kits of the invention or the sealed packages of the invention instructs that such pharmaceutical composition is to be administered for the treatment of head and neck cancer. In another embodiment, the head and neck cancer is SCCHN.

In one embodiment, included instructional material of the pharmaceutical kits of the invention or the sealed packages of the invention instructs that such pharmaceutical composition is to be administered for the treatment of prostate cancer. In another embodiment, the prostate cancer is mCRPC.

In one embodiment, included instructional material of the pharmaceutical kits of the invention or the sealed packages of the invention instructs that such pharmaceutical composition is to be administered for the treatment of urothelial cancer.

In any of the above embodiments, the included instructional material of the pharmaceutical kits of the invention or the sealed packages of the invention may instruct that the pharmaceutical composition is to be administered for treatment of such cancer wherein such cancer is a metastatic cancer. In some embodiments, the included instructional material of the pharmaceutical kits of the invention or the sealed packages of the invention may instruct that the pharmaceutical composition is to be administered for treatment of such cancer wherein such cancer is a primary cancer.

The included instructional material of the pharmaceutical kits of the invention or the sealed packages of the invention may instruct that the pharmaceutical composition is to be administered for treatment of such cancer before, during, or after another treatment for such cancer. In certain of such embodiments, such instructional material may instruction that the pharmaceutical composition is to be administered as a neoadjuvant therapy for treatment of such cancer. In other of such embodiments such instructional material may instruction that the pharmaceutical composition is to be administered as an adjuvant therapy for treatment of such cancer. In other of such embodiments such instructional material may instruction that the pharmaceutical composition is to be administered as a component of a combination therapy for treatment of such cancer.

The included instructional material of the pharmaceutical kits of the invention or the sealed packages of the invention may instruct pharmaceutical composition is to be administered for treatment of such cancer expressing B7-H3. The instructional material may further specify a particular assay or expression measurement, for example expression of B7-H3 by immunohistochemistry. The instructional material may further specify that such B7-H3 expression score is determined by a test approved for use by a regulatory agency (e.g., FDA-approved).

### IV. Methods of Administration

The pharmaceutical compositions of the present invention may be provided for the treatment, prophylaxis, and amelioration of one or more symptoms associated with a disease, disorder or infection by administering to a subject a therapeutically effective amount or prophylactically effective amount of enoblituzumab. In one embodiment, such pharmaceutical compositions are substantially purified (i.e., substantially free from substances that limit its effect or produce undesired side effects) as determined by any suitable method. In another embodiment, the subject is an animal, including a mammal such as non-primate (e.g., bovine, equine, feline, canine, rodent, etc.) or a primate (e.g., monkey such as, a cynomolgus monkey, human, etc.). In one embodiment, the subject is a human. The terms “subject” and “patient” are used herein interchangeably.

As used herein, “a therapeutically effective amount” of enoblituzumab in a pharmaceutical composition of the invention when used for the treatment of a cancer is an amount which can slow the progression of the cancer; reduce the number of cancer cells in fluids (e.g., blood, peripheral cells or lymphatic fluids), tissue or organs (cytoxic); allow the number of cancer cells to remain relatively constant (cytostatic); reduce tumor size, inhibit metastasis, inhibit tumor growth and/or ameliorate one or more of the symptoms of the cancer. Therapeutically effective amounts of enoblituzumab for use in formulating the pharmaceutical compositions of the disclosure are provide herein and/or can be determined, for example, by a health care professional taking into account certain factors such as the type of cancer treated, the route of delivery, the age, weight, severity of the subject's symptoms and response pattern of the subject. As used herein, a “prophylactically effective amount” of enoblituzumab in a pharmaceutical composition of the disclosure when used for the prophylaxis of a cancer is an amount which can prevent or reduce the risk of occurrence or recurrence of the cancer. As used herein, treatment of a cancer with the pharmaceutical compositions, containers, sealed packages, kits or methods of the disclosure, for example, can comprise or can comprise administering a therapeutically effective amount or prophylactically effective amount of enoblituzumab to subject in need thereof.

Methods of administering a pharmaceutical composition (i.e., an enoblituzumab composition) of the invention include, but are not limited to, parenteral administration (e.g., intravenous). In another embodiment, the pharmaceutical composition (i.e., an enoblituzumab composition) of the invention is administered intravenously. The pharmaceutical compositions of the invention may be administered together with other pharmaceutically active agents, such as chemotherapeutic agents, including but not limited to, antimetabolite chemotherapeutics (including pemetrexed), platinum-based chemotherapeutics (including for example, cisplatin and carboplatin), and taxane-based chemotherapeutics (including for example paclitaxel, and nab-paclitaxel), biologic agents, including but not limited to antibodies, and antibody-like molecules including those that bind a cancer antigen or an antigen on immune cells, such as T cells. Such cancer antigens include, but are not limited to, 5T4, CD19, CD20, CD51, CD123, DR5, EGFR, EpCam, GD2, gpA33, HER2, PD-L1, ROR-1, TAG-72, VEGF-A and/or VEGFR2. Such antigens on immune cells include, but are not limited to, CTLA-4, LAG-3 and PD-1. Numerous antibody and antibody-like molecules that bind to such cancer antigens, or antigens on immune cells, have been described and include but are not limited to, bevacizumab, cetuximab, enoblituzumab, flotetuzumab, margetuximab, ofatumumab, panitumumab, retifanlimab, rituximab, tebotelimab, trastuzumab, and others.

In one embodiment, the amount of the pharmaceutical composition (i.e., an enoblituzumab composition) of the invention is determined using a weight-base dose of enoblituzumab to provide a subject with a therapeutically effective amount of prophylactically effective amount of enoblituzumab. The term “weight-based dose” as used herein, refers to a discrete amount of enoblituzumab to be administered per a unit of patient weight, for example milligrams of enoblituzumab per kilograms of a subject's body weight (mg/kg body weight, abbreviated herein as “mg/kg”). The calculated dose is administered based on the subject's body weight at baseline. Typically, a significant (for example at least about a plus or minus 10%) change in body weight from baseline or established plateau weight will prompt recalculation of dose. Single or multiple dosages may be given.

In certain embodiments, enoblituzumab is administered to a subject in need thereof at a weight-based dose of from about 6 mg/kg to about 15 mg/kg. In certain embodiments, enoblituzumab is administered to a subject in need thereof at a dose of 6 mg/kg. In certain embodiments, enoblituzumab is administered to a subject in need thereof at a dose of 10 mg/kg. In certain embodiments, enoblituzumab is administered to a subject in need thereof at a dose of 15 mg/kg. With respect to weight-based doses, the term “about” is intended to denote a range that is ±10% of a recited dose, such that for example, a dose of about 15 mg/kg is 13.5 mg/kg, 16.5 mg/kg, or between 13.5 mg/kg and 16.5 mg/kg.

A dose of the pharmaceutical compositions of the invention (i.e., a dose of an enoblituzumab composition) can be administered to the subject at periodic intervals over a period of time sufficient to encompass at least 2 doses, at least 4 doses, at least 6 doses, at least 12 doses, or at least 24 doses, or more than 24 doses. Such administration of pharmaceutical compositions of the invention at periodic intervals over a period of time can be considered a “course of treatment”. For example, a dosage may be administered e.g., once every two weeks (“Q2W”), once every three weeks (“Q3W”), once every four weeks (“Q4W”), or for shorter or longer periods of time. Such periodic administration may continue for a period of time e.g., for between about 1 to about 52 weeks, or for more than 52 weeks. Such course of treatment may be divided into increments, each referred to herein as a “cycle,” of varying shorter intervals, e.g., between 2 to 8 weeks, during which a set number of doses are administered. The dose and/or the frequency of administration may be the same or different during each cycle. Factors that may influence the dosage and timing required to effectively treat a subject, include, e.g., the severity of the disease or disorder, formulation, route of delivery, previous treatments, the general health and/or age of the subject, and the presence of other diseases in the subject. Moreover, treatment of a subject with a therapeutically effective amount of enoblituzumab can include a single treatment or can include a series of treatments.

A “dosing regimen” is a dosage administration in which a subject is administered a predetermined dose (or set of such predetermined doses) at a predetermined frequency (or set of such frequencies) for a predetermined periodicity (or periodicities). One dosing regimen of the invention comprises administration of an enoblituzumab composition of the invention to a subject at a dose of about 3 mg/kg administered Q3W. Another dosing regimen of the invention comprises administration of an enoblituzumab composition of the invention to a subject at a dose of about 6 mg/kg administered Q3W. Another dosing regimen of the invention comprises administration of an enoblituzumab composition of the invention to a subject at a dose of about 10 mg/kg administered Q3W. Another dosing regimen of the invention comprises administration of an enoblituzumab composition of the invention at a dose of about 15 mg/kg administered Q3W.

It is specifically contemplated that in certain embodiments of the invention, administration of the pharmaceutical composition to a subject occurs at the predetermined frequency or periodicity, or within about 1-3 days of such scheduled interval, such that administration occurs 1-3 day before, 1-3 days after, or on the day of a scheduled dose, e.g., once every 3 weeks (±3 days). In such embodiments, the enoblituzumab composition is administered to a subject in a syringe by infusion pump. In certain embodiments, the enoblituzumab composition is administered by syringe pump infusion according to any of the dosing regimens of the invention for a duration of at least 1 month or more, at least 3 months or more, at least 4 months, at least 6 months or more, or at least 12 months or more than 12 months. In certain embodiments, the enoblituzumab composition is administered by IV infusion. In certain embodiments, the pharmaceutical compositions of the invention are administered by IV infusion which can be continuous intravenous infusion, or discontinuous intravenous infusion. In certain embodiments, the enoblituzumab composition is administered by IV infusion according to any of the above dosing regimens for a duration (i.e., course of treatment) of at least 1 month or more, at least 3 months or more, at least 4 months, at least 6 months or more, or at least 12 months or more than 12 months. A treatment duration of at least 6 months or more, or for at least 12 months or more than 12 months, or, for example, until remission of disease or unmanageable toxicity is observed. In certain embodiments, treatment continues for a period of time after remission of disease. In certain embodiments, treatment may be paused due to illness, adverse event, etc., and is resumed upon resolution, reduction, or amelioration of such illness, adverse event, etc.

In certain embodiments of the methods of the invention, the pharmaceutical composition of the invention (i.e., the enoblituzumab composition) is diluted into a syringe comprising a suitable diluent, e.g., 0.9% sodium chloride or D5W for administration by infusion pump. In certain embodiments, the pharmaceutical composition of the invention is diluted into an infusion bag comprising a suitable diluent, e.g., 0.9% sodium chloride or D5W for administration by IV infusion. Since infusion or allergic reactions may occur, premedication for the prevention of such infusion reactions can be utilized and precautions for anaphylaxis can be observed during the antibody administration.

### V. Administration of Dosing Solutions Comprising a Pharmaceutical Composition

A dosing solution that comprises a pharmaceutical composition (such as an enoblituzumab composition of the invention) is particularly suitable for intravenous administration, for example by gravity or using a stationary infusion pump. A enoblituzumab composition of the invention can be combined with 0.9% sodium chloride or D5W to obtain an enoblituzumab dosing solution. In certain embodiments, the administration of the therapeutic dosage is over at least about 30 minutes or at least about 60 minutes. In certain embodiments, the administration of the therapeutic dosage is over at least about 120 minutes.

In some embodiments, a weight-based dose of about 6 mg/kg to about 15 mg/kg is administered to the patient or subject. In one embodiment, a weight-based dose of about 6 mg/kg, about 10 mg/kg, or about 15 mg/kg is administered to the patient or subject. In other embodiment, a weight-based dose of about 6 mg/kg to about 15 mg/kg is administered Q3W. In other embodiments, a weight-based dose of about 6 mg/kg is administered Q3W. In other embodiments, a weight-based dose of about 10 mg/kg is administered Q3W. In other embodiments, a weight-based dose of about 15 mg/kg is administered Q3W.

In another embodiment, the administration of such doses is by IV infusion over at least about 30 minutes or over at least about 120 minutes. In another embodiment, the administration of such doses is by IV infusion over at least about 30 minutes or over at least about 90 minutes. In another embodiment, the administration of such doses is by IV infusion over at least about 30 minutes or over at least about 60 minutes. In another embodiment, the administration of the enoblituzumab dosing solution is by IV infusion for at least about 30 minutes. In another embodiment, the administration of the enoblituzumab dosing solution is by IV infusion for at least about 60 minutes. In another embodiment, the administration of the enoblituzumab dosing solution is by IV infusion for at least about 120 minutes.

To form a dosing solution, the pharmaceutical composition (i.e., a enoblituzumab composition of the invention) can be added to a container, such as an IV bag, containing for example 0.9% sodium chloride or D5W (nominal volume of 100 mL or 250 mL). In another embodiment, the pharmaceutical composition of the invention may be added to a container, such as a syringe containing 0.9% sodium chloride or D5W (nominal volume 20 mL). In one embodiment, the pharmaceutical composition of the invention is swirled gently prior to being added to a container containing 0.9% sodium chloride or D5W. In one embodiment, the container is an IV bag. In one embodiment, the IV bag is a polyvinyl chloride (PVC) bag, a polyolefin copolymer (polypropylene and polyethylene) bag, a PVC bag containing a Di-2-ethyhexyl phthalate (DEHP), a polyolefin bag with polyamide coating, or an ethylene vinyl acetate (EVA) bag. In another embodiment, the container is a syringe. In one embodiment, the syringe is a polypropylene syringe. In one embodiment, an in-line filter is used during administration. In one embodiment, the filter has a pore size of 0.2 μm, 5 μm or 15 μm. In one embodiment, a 0.2 μm pore size line-line filter is used. In one embodiment, the filter is a polyvinylidene fluoride or cellulose acetate filter. In one embodiment, the filter is a polyethersulfone (PES) filter. In some embodiments, the desired volume of the pharmaceutical composition of the invention is added to the IV bag or syringe and can, for example, be gently inverted to mix the dosing solution.

In one embodiment, the prepared dosing solution is used immediately. In another embodiment, the prepared dosing solution is stored at 25° C. for up to about 4 hours or at about 2° C. to about 8° C. for up to about 24 hours. In another embodiment, the prepared dosing solution that is stored at about 2° C. to about 8° C. for up to about 24 hours is stored at room temperature for a about 30 to about 60 minute equilibration period prior to administration.

### VI. Uses of the Compositions of the Invention

The pharmaceutical compositions, containers, sealed packages, and kits of the invention can be used in methods for the treatment of a cancer, and in certain embodiments, for the treatment of a cancer expressing B7-H3, for example in a therapeutically effective amount or a prophylactically effective amount. In some embodiments, the methods of the invention comprise the step of administering a pharmaceutical composition of the invention to a subject in need thereof for the treatment of cancer, for example in a therapeutically effective amount or a prophylactically effective amount. In some embodiments, cancers to be treated with pharmaceutical compositions, containers, sealed packages or kits of the invention are selected from the group consisting of: adrenal gland cancer, AIDS-associated cancer, alveolar soft part sarcoma, anal cancer (including squamous cell carcinoma of the anal canal (SCAC)), bladder cancer, bone cancer, brain and spinal cord cancer, breast cancer (including, HER2⁺ breast cancer or Triple-Negative Breast Cancer (TNBC)), carotid body tumor, cervical cancer (including, HPV-related cervical cancer), chondrosarcoma, chordoma, chromophobe renal cell carcinoma, clear cell carcinoma, colon cancer, colorectal cancer, desmoplastic small round cell tumor, ependymoma, endometrial cancer (including, unselected endometrial cancer, MSI-high endometrial cancer, dMMR endometrial cancer, and/or POLE exonuclease domain mutation positive endometrial cancer), Ewing's sarcoma, extraskeletal myxoid chondrosarcoma, gallbladder or bile duct cancer (including, cholangiocarcinoma bile duct cancer), gastric cancer, gastroesophageal junction (GEJ) cancer, gestational trophoblastic disease, germ cell tumor, glioblastoma, head and neck cancer (including, squamous cell carcinoma of head and neck (SCCHN)), a hematological malignancy, a hepatocellular carcinoma, islet cell tumor, Kaposi's Sarcoma, kidney cancer, leukemia (including, acute myeloid leukemia), liposarcoma/malignant lipomatous tumor, liver cancer (including, hepatocellular carcinoma liver cancer (HCC)), lymphoma (including, diffuse large B-cell lymphoma (DLBCL), non-Hodgkin's lymphoma (NHL)), lung cancer (including, small cell lung cancer (SCLC), non-small cell lung cancer (NSCLC)), medulloblastoma, melanoma (including, uveal melanoma), meningioma, Merkel cell carcinoma, mesothelioma (including, mesothelial pharyngeal cancer), multiple endocrine neoplasia, multiple myeloma, myelodysplastic syndrome, neuroblastoma, neuroendocrine tumors, ovarian cancer, pancreatic cancer, papillary thyroid carcinoma, parathyroid tumor, pediatric cancer, peripheral nerve sheath tumor, pharyngeal cancer, pheochromocytoma, pituitary tumor, prostate cancer (including, metastatic castration resistant prostate cancer (mCRPC)), posterious uveal melanoma, renal metastatic cancer, rhabdoid tumor, rhabdomyosarcoma, sarcoma, skin cancer, a small round blue cell tumor of childhood (including neuroblastoma and rhabdomyosarcoma), soft-tissue sarcoma, squamous cell cancer, stomach cancer, synovial sarcoma, testicular cancer, thymic carcinoma, thymoma, thyroid cancer, urothelial cancer, and uterine cancer. The included instructional material of the pharmaceutical kits of the invention may instruct that such pharmaceutical composition is to be administered for a cancer selected from the group consisting of: anal cancer, bladder cancer, breast cancer, bile duct cancer, cervical cancer, colorectal cancer, endometrial cancer, gastric cancer, GEJ cancer, head and neck cancer, liver cancer, lung cancer, lymphoma, melanoma, ovarian cancer, prostate cancer and urothelial cancer.

In one embodiment, a pharmaceutical composition, container, sealed package, or kit of the invention is used for the treatment of anal cancer. In another embodiment, the anal cancer is SCAC.

In one embodiment, a pharmaceutical composition, container, sealed package, or kit of the invention is used for the treatment of lung cancer. In another embodiment, the lung cancer is NSCLC.

In one embodiment, a pharmaceutical composition, container, sealed package, or kit of the invention is used for the treatment of skin cancer. In another embodiment, the skin cancer is melanoma. In another embodiment, the skin cancer is Merkel cell carcinoma.

In one embodiment, a pharmaceutical composition, container, sealed package, or kit of the invention is used for the treatment of head and neck cancer. In another embodiment, the head and neck cancer is SCCHN.

In one embodiment, a pharmaceutical composition, container, sealed package, or kit of the invention is used for the treatment of prostate cancer. In another embodiment, the prostate cancer is mCRPC.

In one embodiment, a pharmaceutical composition, container, sealed package, or kit of the invention is used for the treatment of urothelial cancer.

In certain embodiments, a pharmaceutical composition, container, sealed package, or kit of the invention is used for treatment of such cancer wherein such cancer is a metastatic cancer. In certain embodiments, a pharmaceutical composition, container, sealed package, or kit of the invention is used for treatment of such cancer wherein such cancer is a metastatic cancer.

In certain embodiments, a pharmaceutical composition, container, sealed package, or kit of the invention is used as a neoadjuvant therapy for treatment of such cancer. In certain embodiments, a pharmaceutical composition, container, sealed package, or kit of the invention is used as an adjuvant therapy for treatment of such cancer. In other embodiments, a pharmaceutical composition, container, sealed package, or kit of the invention is used as a component of a combination therapy for treatment of such cancer.

In certain embodiments, a pharmaceutical composition, container, sealed package, or kit of the invention is used for treatment of such cancer, wherein such cancer expresses B7-113.

### EXAMPLES

Having now generally described the invention, the same will be more readily understood through reference to the following examples, which are provided by way of illustration and are not intended to be limiting of the present invention unless specified.

### Example 1

### Development of a Pharmaceutical Composition Containing Enoblituzumab

A stable antioxidant-free pharmaceutical composition comprising enoblituzumab (the “enoblituzumab drug product (DP) composition”), in a liquid composition in vials was prepared.

### 1.1. Target Product Profile of Exemplary Enoblituzumab DP Compositions

The target product profile of an exemplary enoblituzumab DP composition, for 250 mg or 425 mg vials, is shown below in Table 1.

TABLE 1
Target Product Profile of an Exemplary Enoblituzumab DP Composition
for 125 mg, 250 mg, 425 mg, 500 mg, and 1200 mg vials

| Product Attribute | Target |
|---|---|
| Dosage form | Injection: Sterile aqueous solution |
| Protein content per vial | >125 mg, ≥250 mg, ≥425 mg, ≥500 mg, |
| | or ≥1,200 mg |
| Dose | 250 mg or 425 mg or 1,200 mg |
| Protein Concentration | ≥5 mg/mL (up to about 200 mg/mL) |
| Shelf life | ≥24 months at 2-8° C. |
| Degradants/impurities | Below safety threshold |
| Aggregates | <5% increase |

### 1.2. Development of an Enoblituzumab Drug Product (DP) Composition in 10 mM Acetate

Initially, enoblituzumab was formulated in 0.15 M NaCl and 0.05 mg/mL polysorbate-80 (PS80) in 10 mM sodium phosphate at pH 6.1 (PBS-T), a formulation that is acceptable for many IgG1 antibodies. However, this condition resulted in a turbid appearance when the enoblituzumab concentrate was refrigerated (2-8° C.). Various protein concentrations (21.1 mg/mL to 39.4 mg/mL; as shown in Table 2) were tested to determine the effects of buffer, pH, excipients and surfactant on the visual appearance of the formulation at 2-8° C.

TABLE 2
Appearance of Enoblituzumab DP Formulations at 2-8° C.

| | Enoblituzumab | |
|---|---|---|
| Formulation | Concentration | Visual Appearance |
| (% = W/V) | (mg/mL) | at 2-8° C. |
| PBS-T, pH 6.1 | 29.3 | turbid |
| pH 5.5, 10 mM Na acetate, 0.05M | 24.1 | slight hazy |
| NaCl | | |
| pH 5.1, 10 mM Na acetate, 0.05M | 21.1 | clear |
| NaCl | | |
| pH 5.1, 10 mM Na acetate, 0.1 | 38.1 | clear |
| mg/mL PS80 | | |
| pH 6.1, 10 mM Na phosphate, 0.1 | 39.4 | clear |
| mg/mL PS80 | | |
| pH 5.1, 10 mM Na acetate, 9% | 25.1 | clear |
| sucrose, 0.1 mg/mL PS80 | | |
| pH 5.1, 10 mM Na acetate, 9% | 24.5 | clear |
| sucrose, 0.1 mg/mL PS80 | | |

The stability of enoblituzumab was compared between pH 5.1 (10 mM acetate buffer), pH 5.5 (10 mM acetate buffer) and pH 6.1 (10 mM phosphate buffer) in the presence of sucrose, sodium chloride and polysorbate 80 (PS80). As shown in Table 2, improved solubility properties for enoblituzumab resulted from either lowering the pH and/or eliminating the NaCl. Additionally, flux rates during diafiltration at pH 5.1 and pH 6.1, showed a significantly higher exchange efficiency for the pH 5.1 condition. The difference in diafiltration flux was consistent with a higher solubility for enoblituzumab at pH 5.1. Additional evaluations of diafiltered enoblituzumab concentrates in the presence of 10 mM sodium acetate at pH 5.1 established its compatibility and molecular stability, with no observable change in monomeric content. This formulation yielded a clear solution of antibody that was readily filtered through 0.2 μm membranes. To suppress the formation of subvisible particles, which are often induced through agitation of protein solutions, 0.1 mg/mL PS80 was added to filtered enoblituzumab. Thus, in this example, 10 mM sodium acetate buffer (3 mM glacial acetic acid, 7 mM sodium acetate) pH 5.1 and 0.1 mg/mL PS80 was selected as a formulation buffer for further development.

### 1.2.1. Evaluation of Excipients

Additional, studies were conducted to identify excipients capable of enhancing the stability of enoblituzumab upon storage (e.g., refrigerator or frozen storage). Enoblituzumab formulations, containing 10 mM sodium acetate and 0.1 mg/mL PS-80 at pH 5.1, or 10 mM sodium phosphate and 0.1 mg/mL PS-80 at pH 6.1, were further formulated by addition of various excipients. One pH 6.1 formulation and seven other formulations at pH 5.1 were prepared by adding combinations of sucrose, trehalose, sorbitol, arginine, lysine, glutamine and NaCl to obtain nearly iso-osmolal mixtures. Enoblituzumab product stability in these formulations was evaluated under different storage temperatures, agitation stress at room temperature, and under freeze thaw conditions. The eight enoblituzumab formulations tested are shown in Table 3, and the study results are provided below.

### 1.2.2. Evaluation of Visual Appearance

The impact of selected excipients was first evaluated by visual appearance of the mixtures at 2-8° C. Solutions of enoblituzumab at either pH 5.1 (acetate) or pH 6.1 (phosphate) became cloudy with the addition of 50 mM NaCl, 50 mM arginine and 3% sucrose (see #5, #6 in Table 3). Other combinations of basic amino acids with 3-6% sucrose also remained hazy after mixing (see #3, #7, #8 in Table 3). Clear appearance was only achieved for the salt-free and arginine-free conditions (see #1, #2, #4 in Table 3).

TABLE 3
Effects of Formulation Excipients and pH on Visual Appearance,
Cryostability and Aggregation of Enoblituzumab

| | | | | Freeze-Thaw | 37° C. |
|---|---|---|---|---|---|
| | Formulation | | | (% Aggregate | (% Aggregate |
| | Excipient(s) | | Appearance | Change after | Change after |
| # | (% = W/V) | pH | at 2-8° C. | 1 & 5 cycles) | 2 & 4 weeks)^{c} |
| 1^{a} | 9% sucrose | 5.1 | clear | 0.16, −0.18 | 0.12, 0.87 |
| 2^{a} | 10% trehalose | 5.1 | clear | −0.07, 0.45 | 0.36, 0.38 |
| 3^{a} | 6% sucrose, 0.05M | 5.1 | hazy | −0.09, −0.09 | 0.67, 1.05 |
| | arginine | | | | |
| 4^{a} | 6% sucrose, 1.5% | 5.1 | clear | −0.16, −0.18 | 0.27, 0.76 |
| | sorbitol | | | | |
| 5^{a} | 3% sucrose, 0.05M | 5.1 | cloudy | −0.07, 0.05 | 0.70, 1.39 |
| | arginine, 0.05M NaCl | | | | |
| 6^{b} | 3% sucrose, 0.05M | 6.1 | cloudy | 0.04, 0.04 | 0.9, 1.32 |
| | arginine, 0.05M NaCl | | | | |
| 7^{a} | 3% sucrose, 0.05M | 5.1 | hazy | 0.37, 0.59 | 1.24, 3.25 |
| | lysine, 0.05M NaCl | | | | |
| 8^{a} | 3% sucrose, 0.05M | 5.1 | hazy | −0.04, 0.12 | 0.65, 2.16 |
| | glutamine, 0.05M NaCl | | | | |

^{a}10 mM sodium acetate and 0.1 mg/mL PS-80 base formulation
^{b}10 mM sodium phosphate and 0.1 mg/mL PS-80 base formulation
^{c}Aggregate formation is not linear over time; reported rate is incremental value

### 1.2.3. Freeze Thaw Study

A freeze/thaw study was performed to assess the impact of freezing at 80° C. and thawing at 25° C. for five cycles on stability of enoblituzumab in 7 exemplary formulations at pH 5.1 and one exemplary formulation at pH 6.1. The samples were analyzed at initial and after 1 and 5 cycles for appearance, % aggregates by size exclusion high-performance liquid chromatography (“SE-HPLC”). Cryoprotection was similar for all formulations except those containing trehalose at pH 5.1 (#2) and the combination of sucrose-lysine-NaCl at pH 5.1 showed slightly higher levels of aggregate formation after repeated freeze and thaw (#7). The 9% sucrose formulation was one of two formulations out of eight tested that was most compatible in both appearance and freeze-thaw protection against aggregation. The analytical results are summarized in Table 3.

The results obtained show that significantly more sub-visible particles formed in the presence of sodium chloride after freeze/thaw. From the freeze/thaw study, 9% (90 mg/mL) sucrose, 0.01% (0.1 mg/mL) PS80, 10 mM acetate pH 5.1 was the most stable of the exemplary formulations for enoblituzumab.

### 1.2.4. Aggregation Study

Accelerated stability studies were subsequently conducted for enoblituzumab in glass vials at an elevated storage temperature of 37° C. as shown in Table 3. These conditions artificially enhance the rate of aggregate formation and fragmentation, which may help differentiate the potential stabilizing properties of the added excipients. The arginine-sucrose combination (#3) and the four NaCl containing formulations (#5, #6, #7, #8) appeared to destabilize enoblituzumab at the elevated temperature. The addition of 9% sucrose (#1) relative to other iso-osmolal formulations achieved a consistent improvement in appearance and stability. Thus, aggregation studies also confirmed that the formulation comprising 10 mM sodium acetate at pH 5.1 containing 9% sucrose and 0.1 mg/mL PS80 was the most stable of the exemplary formulations for enoblituzumab.

### 1.3. Development of an Enoblituzumab Drug Product (DP) Composition in 20 mM Acetate

Studies were performed to evaluate additional exemplary formulations, particularly antioxidant-free acetate formulations for enoblituzumab concentrations of ≥60 mg/mL.

### 1.3.1. pH Shift Analysis

For this study 20 mM sodium acetate buffers having a pH of 3.7-5.0 were chosen to evaluate pH shift at the higher concentration of 60 mg/mL. Briefly, unformulated enoblituzumab was buffer exchanged two times into 20 mM formulation buffers having a starting pH of 3.7, 4.3, 4.5, 4.8, or 5.0, using dialysis cassettes. The resulting material was then concentrated to 60 mg/mL and sucrose (final 9%) and PS80 (final 0.1%) were added. The pH, protein concentration (Solo-VPE), and percent high molecular weight species (% HMWS) by SE-HPLC were measured to evaluate the pH shift and aggregates level of each formulation. The results are shown in Table 4.

TABLE 4
Enoblituzumab pH Shift Results

| | Actual | Protein | |
|---|---|---|---|
| Formulation Starting Buffer | pH after | Conc. | % HMWS by |
| Conditions for Dialysis | Dialysis | (mg/mL) | SE-HPLC |
| 20 mM Sodium Acetate, 9% | 4.5 | 60.7 | 1.7 |
| Sucrose, 0.01% PS80, pH 3.7 | | | |
| 20 mM Sodium Acetate, 9% | 4.8 | 61.2 | 1.8 |
| Sucrose, 0.01% PS80, pH 4.3 | | | |
| 20 mM Sodium Acetate, 9% | 4.9 | 61.4 | 1.8 |
| Sucrose, 0.01% PS80, pH 4.5 | | | |
| 20 mM Sodium Acetate, 9% | 5.0 | 61.1 | 1.8 |
| Sucrose, 0.01% PS80, pH 4.8 | | | |
| 20 mM Sodium Acetate, 9% | 5.2 | 60.8 | 2.0 |
| Sucrose, 0.01% PS80, pH 5.0 | | | |

All formulations were observed to have a pH shift after buffer exchange into target formulation. The pH shift is more significant when the starting buffer pH is further from the molecule pI of 8.65. A 0.8 pH unit shift for pH buffer starting at 3.7 resulting in a pH 4.5 formulation was observed and the pH shift is reduced to 0.2 for when using starting pH buffer at pH 5.0 that resulted in a pH of 5.2 in the final formulation. Enoblituzumab at >60 mg/mL formulated in 20 mM sodium acetate buffer at a lower pH of 4.5 had the lowest % HMWS of 1.7% compared to formulation condition at pH 5.2 with the highest % HMWS of 2.0%. The % HMWS results indicated a slight pH dependency for aggregate formation with lower % HMWS observed in lower pH formulations.

The pH shift effects are observed because enoblituzumab is a highly charged antibody with a pI of pH 8.65, as determined by imaged capillary isoelectric focusing (iCIEF). This study demonstrated that formulations of enoblituzumab, particularly high concentration formulations, are subject to shifts in pH. The pH shift progressively decreased with increasing pH. Also, the formation of aggregates is partially dependent on the pH as lower % aggregates were observed in lower pH conditions. Thus, use of a higher buffer concentration (e.g., 20 mM) capable of better achieving and maintaining the optimal pH will enhance the long-term stability of high concentration enoblituzumab formulations.

### 1.3.2. Accelerated and Stressed Thermal Study

A short term stability study was conducted to monitor enoblituzumab stability in additional exemplary enoblituzumab formulations under frozen storage conditions (−60° C. to −80° C.), normal storage conditions (2-8° C.), accelerated storage conditions (23-27° C.) and stressed storage conditions (38-42° C.). Two 20 mM acetate high concentration formulations comprising 60 mg/mL enoblituzumab, and a 10 mM acetate low concentration formulation comprising 25 mg/mL formulation were evaluated in this study, the formulation components, target protein concentrations and pH are provided in Table 5. The enoblituzumab formulations were prepared and sterile filtered using a 0.22 μM SterileFlip (PVDF) filter prior to filling into sterile 2 mL USP Type 1 glass vials. Each vial was stoppered and capped under aseptic conditions. The vials were stored at the frozen (−60° C. to −80° C.), normal (2-8° C.), accelerated (23-27° C.), and stressed (38-42° C.) storage conditions. The product quality of enoblituzumab was evaluated using visual inspection, protein concentration (UV 280 nm using Solo-VPE Spectrophotometer), pH, osmolality, subvisible particulates (light obscuration, HIAC, selected time points), % high molecular weight species (% HMW) (SE-HPLC) and charge variants distribution (IE-HPLC). The stability study matrix including time points and storage conditions are summarized in Table 6.

TABLE 5
Short Term Stability Study Formulation List

| | | | | Protein | |
|---|---|---|---|---|---|
| Formulation | | % Sucrose | % PS80 | Conc. | Buffer |
| Code | Buffer | (w/v) | (w/v) | (mg/mL) | pH |
| 20A48-60 | 20 mM Sodium | 9% | 0.01% | 60 | 4.8 |
| | Acetate | | | | |
| 20A51-60 | 20 mM Sodium | 9% | 0.01% | 60 | 5.1 |
| | Acetate | | | | |
| 10A50-25 | 10 mM Sodium | 9% | 0.01% | 25 | 5.0 |
| | Acetate | | | | |

TABLE 6
Enoblituzumab Formulation Stability Study Matrix

| | Time | | | | | | |
|---|---|---|---|---|---|---|---|
| | | 1 | 2 | 4 | 2 | 3 | 6 |
| Temperature | 0 | Week | Weeks | Weeks | Months | Months | Months |
| −70 ± 2° C. | | | | X | | X | X |
| 5 ± 3° C. | X* | | | X | X | X | X* |
| 25 ± 2° C. | | | X | X | | X | X |
| 40 ± 2° C. | | X | X | X | | | |

*Sub-visible particle counts by HIAC was performed for this time point.

The stability study results are summarized in Table 7 to Table 15. All three formulations were observed to be clear, pale yellow, essentially free from visible foreign particles and proteinaceous particles. Protein concentration, pH and osmolality measured at T=0 are shown in Table 7. Enoblituzumab formulated in 10 mM sodium acetate at pH 5 with a concentration of 25 mg/mL (10A50-25) showed no increase in % HMW species at all storage condition for up to 6 months. Enoblituzumab formulated in 20 mM sodium acetate at the higher concentration of 60 mg/mL at pH 4.8 (20A48-60) and 5.1 (20A51-60) showed no significant increases in % HMW species when stored under frozen conditions (−70±10° C.) and normal storage conditions (5±3° C.) for up to 6 months (Table 8 and Table 9). Some increase in % HMW species was observed for the high concentration (60 mg/mL) formulations in accelerated (25±2° C.) and stressed (40±2° C.) conditions (Table 10 and Table 11). The high concentration formulation at pH 4.8 (20A48-60) showed slightly lower % HMW species formation compared to the high concentration formulation at pH 5.1 (20A51-50). The high concentration formulation at pH 4.8 showed an increase of 0.3% (1.1% to 1.4%) at 25±2° C. after 3 months, and an increase of 0.4% (1.1% to 1.5%) at 40±2° C. after 4 weeks. In comparison, the high concentration formulation at pH 5.1 showed an increase of 0.4% (1.1% to 1.5%) 0.6% (1.1% to 1.7%), respectively, at the same storage conditions.

The trending of charged variants distribution by IE-HPLC were similar for all three formulations during the 6-month storage. There were no significant changes observed in percent main charge peak (% MCP) when stored at frozen (−70±10° C.) and recommended (5±3° C.) storage conditions for up to 6 months (Table 12 and Table 13). However, % MCP decreased from ˜32% to ˜24% under accelerated storage condition of 25±2° C. after 3 months (Table 14) and decreased from ˜32% to ˜16% under stressed storage condition of 40±2° C. after 4 weeks (Table 15). This decrease was accompanied by an increase in both acidic variants (AV) and basic variants (BV). The charge variants data shows no pH or concentration dependency on degradation trend.

TABLE 7
Short Term Stability Visual and Osmolality Results

| Formulation | Visual^{A} | | | Concentration | | Osmo- |
|---|---|---|---|---|---|---|
| Code | Clarity | Color | Particle | (mg/mL) | pH | lality |
| 20A48-60 | C | PY | FPP, FNP | 61.8 | 4.8 | 328 |
| 20A51-60 | C | PY | FPP, FNP | 59.1 | 5.0 | 348 |
| 10A50-25 | C | PY | FPP, FNP | 25.3 | 5.0 | 331 |

Abbreviations used in Table 5: C = clear; PY = pale yellow; FPP = essentially free from visible proteinaceous particles; FNP = essentially free from visible foreign particles.

TABLE 8
Short Term Stability SE-HPLC results at −70 ± 2° C.

| Formulation | T = 0 | | | 1 Month | | |
|---|---|---|---|---|---|---|
| Code | % H | % M | % L | % H | % M | % L |
| 20A48-60 | 1.1 | 98.8 | 0.1 | 1.0 | 98.9 | 0.1 |
| 20A51-60 | 1.1 | 98.8 | 0.1 | 1.1 | 98.8 | 0.1 |
| 10A50-25 | 1.1 | 98.8 | 0.1 | 1.0 | 98.9 | 0.1 |
| Formulation | 3 Months | | | 6 Months | | |
| Code | % H | % M | % L | % H | % M | % L |
| 20A48-60 | 1.1 | 98.8 | 0.2 | 1.1 | 98.8 | 0.1 |
| 20A51-60 | 1.1 | 98.7 | 0.1 | 1.1 | 98.8 | 0.1 |
| 10A50-25 | 1.1 | 98.8 | 0.2 | 1.1 | 98.8 | 0.2 |

TABLE 9
Short Term Stability SE-HPLC results at 5 ± 3° C.

| Formulation | T = 0 | | | 1 Month | | | 2 Months | | |
|---|---|---|---|---|---|---|---|---|---|
| Code | % H | % M | % L | % H | % M | % L | % H | % M | % L |
| 20A48-60 | 1.1 | 98.8 | 0.1 | 1.0 | 98.8 | 0.1 | 1.1 | 98.7 | 0.2 |
| 20A51-60 | 1.1 | 98.8 | 0.1 | 1.1 | 98.8 | 0.1 | 1.2 | 98.6 | 0.2 |
| 10A50-25 | 1.1 | 98.8 | 0.1 | 1.0 | 98.9 | 0.1 | 1.0 | 98.8 | 0.2 |

| Formulation | 3 Months | | | 6 Months | | |
|---|---|---|---|---|---|---|
| Code | % H | % M | % L | % H | % M | % L |
| 20A48-60 | 1.1 | 98.7 | 0.2 | 1.2 | 98.7 | 0.2 |
| 20A51-60 | 1.2 | 98.6 | 0.2 | 1.2 | 98.6 | 0.2 |
| 10A50-25 | 1.0 | 98.8 | 0.2 | 1.0 | 98.8 | 0.2 |

TABLE 10
Short Term Stability SE-HPLC results at 25 ± 2° C.

| Formulation | T = 0 | | | 2 Weeks | | |
|---|---|---|---|---|---|---|
| Code | % H | % M | % L | % H | % M | % L |
| 20A48-60 | 1.1 | 98.8 | 0.1 | 1.1 | 98.7 | 0.2 |
| 20A51-60 | 1.1 | 98.8 | 0.1 | 1.2 | 98.6 | 0.2 |
| 10A50-25 | 1.1 | 98.8 | 0.1 | 1.0 | 98.9 | 0.1 |
| Formulation | 1 Month | | | 3 Months | | |
| Code | % H | % M | % L | % H | % M | % L |
| 20A48-60 | 1.1 | 98.6 | 0.3 | 1.4 | 98.1 | 0.6 |
| 20A51-60 | 1.3 | 98.5 | 0.2 | 1.5 | 98.0 | 0.5 |
| 10A50-25 | 1.0 | 98.8 | 0.2 | 1.1 | 98.5 | 0.4 |

TABLE 11
Short Term Stability SE-HPLC results at 40 ± 2° C.

| Formula- | T = 0 | | | 1 Week | | |
|---|---|---|---|---|---|---|
| tion Code | % H | % M | % L | % H | % M | % L |
| 20A48-60 | 1.1 | 98.8 | 0.1 | 1.3 | 98.4 | 0.3 |
| 20A51-60 | 1.1 | 98.8 | 0.1 | 1.4 | 98.4 | 0.2 |
| 10A50-25 | 1.1 | 98.8 | 0.1 | 1.0 | 98.7 | 0.2 |
| Formula- | 2 Weeks | | | 4 Weeks | | |
| tion Code | % H | % M | % L | % H | % M | % L |
| 20A48-60 | 1.4 | 98.1 | 0.6 | 1.5 | 97.5 | 1.0 |
| 20A51-60 | 1.5 | 98.0 | 0.5 | 1.7 | 97.5 | 0.8 |
| 10A50-25 | 1.1 | 98.5 | 0.4 | 1.1 | 98.2 | 0.7 |

Abbreviations used in Tables 8-11: H=High molecular Weight Species, M=mono,er, L=Low Molecular Weight Species

TABLE 12
Short Term Stability IE-HPLC results at −70 ± 2° C.

| | T = 0 | | | 1 Month | | |
|---|---|---|---|---|---|---|
| Formula- | % | % | % | % | % | % |
| tion Code | AV | MC | BV | AV | MC | BV |
| 20A48-60 | 24.7 | 32.5 | 42.7 | 24.7 | 32.9 | 42.4 |
| 20A51-60 | 25.2 | 32.1 | 42.7 | 24.6 | 32.7 | 42.7 |
| 10A50-25 | 25.1 | 32.3 | 42.6 | 24.7 | 33.1 | 42.2 |
| | 3 Months | | | 6 Months | | |
| Formula- | % | % | % | % | % | % |
| tion Code | AV | MC | BV | AV | MC | BV |
| 20A48-60 | 25.3 | 31.8 | 43.0 | 24.8 | 32.9 | 42.3 |
| 20A51-60 | 25.1 | 31.8 | 43.1 | 25.0 | 32.3 | 42.7 |
| 10A50-25 | 25.4 | 32.0 | 42.7 | 24.9 | 32.5 | 42.5 |

TABLE 13
Short Term Stability IE-HPLC results at 5 ± 3° C.

| | T = 0 | | | 1 Month | | | 2 Months | | |
|---|---|---|---|---|---|---|---|---|---|
| Formulation | % | % | % | % | % | % | % | % | % |
| Code | AV | MC | BV | AV | MC | BV | AV | MC | BV |
| 20A48-60 | 24.7 | 32.5 | 42.7 | 24.8 | 33.3 | 42.0 | 24.7 | 32.8 | 42.5 |
| 20A51-60 | 25.2 | 32.1 | 42.7 | 24.5 | 33.5 | 42.0 | 25.0 | 32.2 | 42.8 |
| 10A50-25 | 25.1 | 32.3 | 42.6 | 24.7 | 32.4 | 42.9 | 24.9 | 32.1 | 42.9 |

| | 3 Months | | | 6 Months | | |
|---|---|---|---|---|---|---|
| Formulation | % | % | % | % | % | % |
| Code | AV | MC | BV | AV | MC | BV |
| 20A48-60 | 24.9 | 31.7 | 43.4 | 24.6 | 30.9 | 44.5 |
| 20A51-60 | 25.0 | 31.5 | 43.5 | 24.8 | 31.2 | 44.0 |
| 10A50-25 | 25.3 | 31.5 | 43.2 | 24.6 | 31.7 | 43.6 |

TABLE 14
Short Term Stability IE-HPLC results at 25 ± 2° C.

| Formula- | T = 0 | | | 2 Weeks | | |
|---|---|---|---|---|---|---|
| tion Code | % AV | % MC | % BV | % AV | % MC | % BV |
| 20A48-60 | 24.7 | 32.5 | 42.7 | 24.6 | 30.5 | 44.9 |
| 20A51-60 | 25.2 | 32.1 | 42.7 | 23.8 | 31.8 | 44.4 |
| 10A50-25 | 25.1 | 32.3 | 42.6 | 24.0 | 31.7 | 44.4 |
| Formula- | 1 Month | | | 3 Months | | |
| tion Code | % AV | % MC | % BV | % AV | % MC | % BV |
| 20A48-60 | 23.7 | 31.7 | 44.6 | 26.4 | 23.8 | 49.8 |
| 20A51-60 | 23.9 | 31.3 | 44.8 | 27.0 | 24.1 | 48.9 |
| 10A50-25 | 23.9 | 31.2 | 45.0 | 26.6 | 23.9 | 49.4 |

TABLE 15
Short Term Stability IE-HPLC results at 40 ± 2° C.

| Formula- | T = 0 | | | 1 Week | | |
|---|---|---|---|---|---|---|
| tion Code | % AV | % MC | % BV | % AV | % MC | % BV |
| 20A48-60 | 24.7 | 32.5 | 42.7 | 27.4 | 25.6 | 47.0 |
| 20A51-60 | 25.2 | 32.1 | 42.7 | 27.9 | 25.5 | 46.6 |
| 10A50-25 | 25.1 | 32.3 | 42.6 | 27.8 | 25.5 | 46.7 |
| Formula- | 2 Weeks | | | 4 Weeks | | |
| tion Code | % AV | % MC | % BV | % AV | % MC | % BV |
| 20A48-60 | 27.7 | 21.0 | 51.3 | 29.6 | 16.1 | 54.3 |
| 20A51-60 | 27.9 | 21.5 | 50.7 | 30.1 | 16.3 | 53.6 |
| 10A50-25 | 29.6 | 23.4 | 47.0 | 30.2 | 16.8 | 53.1 |

Abbreviations used in Tables 12-15: AV=acidic variants; MC=main charge peak; BV=basic variants

In summary, this short-term stability study demonstrated stability of all three exemplary enoblituzumab formulations at both frozen (−60° C. to −80° C.) and 2-8° C. storage conditions with no significant change in product quality for both molar weight distribution by SE-HPLC and charge variants distribution by IE-HPLC after 6 months of storage. The results of this study show that the high concentration formulation (60 mg/mL, 20 mM sodium acetate buffer, 9% sucrose, and 0.01% PS80) is slightly more stable when formulated at pH 4.8 compared to pH 5.1 based on % HMWS formation rate at accelerated (25±2° C.) and stressed (40±2° C.) conditions. The charge variants distribution showed no concentration or pH dependent degradation at accelerated (25±2° C.) and stressed (40±2° C.) conditions.

### 1.4. Formulation Development Summary

The above formulation development studies demonstrate that enoblituzumab was stable at both frozen (−60° C. to −80° C.) and normal storage conditions (2-8° C.) with no significant change in product quality for both molar weight distribution by SE-HPLC and charge variants distribution by IE-HPLC after 6 months of storage when formulated in the 10 mM acetate formulation (25 mg/mL enoblituzumab, 10 mM sodium acetate, 9% sucrose, and 0.01% PS80 at pH 5.1±0.4) and in the 20 mM acetate formulations (60 mg/mL, 20 mM sodium acetate, 9% sucrose, and 0.01% PS80 at pH 4.8±0.4).

These studies also demonstrate that in contrast to other described antibody compositions, enoblituzumab can be formulated without the use of antioxidants (e.g., histidine, methionine) in an acetate buffer about 10 mM to about 20 mM acetate, comprising sucrose, and PS80 having a pH of 4.4 to 5.5. In particular, these studies support the use of 20 mM acetate, sucrose and PS80 (e.g., 20 mM sodium acetate, 9% sucrose, and 0.01% PS80 at pH 4.8±0.4) as a liquid formulation for high concentration enoblituzumab DP compositions. As provided in more detail below, additional studies were also performed on high concentration enoblituzumab formulations. Based on these formulation development studies, an initial DP composition (25 mg/mL enoblituzumab, 10 mM sodium acetate, 9% sucrose, and 0.01% PS80 at pH 5.1±0.4) was defined (referred to herein as “enoblituzumab DP1”). In addition, a second DP composition (25 mg/mL enoblituzumab, 10 mM sodium acetate, 9% sucrose, and 0.01% PS80 at pH 5.0±0.4) was defined (referred to herein as “enoblituzumab DP2”).

### 1.5. How the Enoblituzumab DP1 Composition is Supplied

The components of the selected enoblituzumab DP1 and DP2 compositions are shown below in Tables 16A and 16B, respectively. The enoblituzumab DP1 and DP2 compositions were supplied in 10 mL or 20 mL Type 1 borosilicate vials as shown in Tables 16A-16B: 250 mg/Co mL (10 mL vial) or 425 mg/17 mL (20 mL vial). DP1 (Table 16A) and DP2 (Table 16B) compositions both comprise 25 mg/mL enoblituzumab, 10 mM sodium acetate, 90 sucrose, and 0.0100 PS80, but have different ion concentrations and slightly different target pHs. The DP2 formulation ensures the right target pH of 5.0 is achieved and maintained during long term stability.

TABLE 16A
Enoblituzumab DP1 Composition (250
mg and 425 mg vials) pH 5.1 ± 0.4

DP

| Composition | DP | | Quantity | | | | | |
|---|---|---|---|---|---|---|---|---|
| Components | Composition | | Each mL | | 10 mL/vial | | 17 mL/vial | |
| Enoblituzumab | 25 | mg/mL | 25 | mg | 250 | mg | 425 | mg |
| Glacial acetic | 0.18 | mg/mL | 0.18 | mg | 1.8 | mg | 3.06 | mg |
| acid | | | | | | | | |
| Sodium acetate | 0.95 | mg/mL | 0.95 | mg | 9.5 | mg | 16.15 | mg |
| trihydrate\hat{ } | | | | | | | | |
| Sucrose (9%) | 90 | mg/mL | 90 | mg | 900 | mg | 1530 | mg |
| Polysorbate 80 | 0.10 | mg/mL | 0.10 | mg | 1.0 | mg | 1.7 | mg |
| (“PS80”) | | | | | | | | |
| (0.01%) | | | | | | | | |

| | | | | |
|---|---|---|---|---|
| Water for | q.s. to | q.s. to | q.s. to | q.s. to |
| Injection | 1 mL | volume | volume | volume |

TABLE 16B
Enoblituzumab DP2 Composition (125 mg, 250
mg, 425 mg, and 500 mg vials) pH 5.0 ± 0.4

DP

| Composition | DP | | Quantity | | | | | | | |
|---|---|---|---|---|---|---|---|---|---|---|
| Components | Composition | | 5 mL/vial | | 10 mL/vial | | 17 mL/vial | | 20 mL/vial | |
| Enoblituzumab | 25 | mg/mL | 125 | mg | 250 | mg | 425 | mg | 500 | mg |
| Glacial acetic | 0.27 | mg/mL | 1.35 | mg | 2.7 | mg | 4.59 | mg | 5.4 | mg |
| acid | | | | | | | | | | |
| Sodium acetate | 0.74 | mg/mL | 3.7 | mg | 7.4 | mg | 12.58 | mg | 14.8 | mg |
| trihydrate\hat{ } | | | | | | | | | | |
| Sucrose (9%) | 90 | mg/mL | 450 | mg | 900 | mg | 1530 | mg | 1800 | mg |
| Polysorbate 80 | 0.10 | mg/mL | 0.5 | mg | 1.0 | mg | 1.7 | mg | 2.0 | mg |
| (“PS80”) | | | | | | | | | | |
| (0.01%) | | | | | | | | | | |

| | | | | | |
|---|---|---|---|---|---|
| Water for | q.s. to | q.s. to | q.s. to | q.s. to | q.s. to |
| Injection | 1 mL | volume | volume | volume | volume |

Enoblituzumab DP1 and DP2 compositions were supplied as a sterile buffered aqueous solution and presented in USP and Ph. Eur. conforming Type I borosilicate 10 mL (250 mg/vial) or 20 mL (425 mg/vial) glass vials capped with a 20 mm FluroTec® and B2-40 coated butyl rubber stoppers. The nominal content of each vial was 10 mL or 17 mL. Each vial was filled with a 0.6 mL overfill of liquid. An overfill was included to ensure sufficient volume for withdrawal of 10 mL (250 mg) and 17 mL (425 mg) of enoblituzumab for dose delivery. The target fill volume, deliverable volume and vial/syringe hold up volumes were determined by extractable volume testing. Enoblituzumab DP1 and DP2 compositions are a clear to slightly opalescent, colorless to pale yellow or pale brown solution. Some proteinaceous enoblituzumab particles may be present. Enoblituzumab DP composition supplied as described in this section was used in the Administration Compatibility and Long-Term and Accelerated Stability Studies described below.

### Example 2

### Enoblituzumab DP1 Composition IV Administration Compatibility Studies

Enoblituzumab DP1 composition is available in a single-dose vial and is administered as an intravenous (IV) infusion following dilution in normal saline (0.9% Sodium Chloride Injection, USP). The dilution is calculated based upon the amount to be administered, for example for a weight-based dose, the patient's body weight and the dose are used to calculate the amount. Compatibility was tested to accommodate a large range of doses: 0.01 mg/kg to 15 mg/kg.

2.1. Overview of In-Use Compatibility Studies with Enoblituzumab DP1

To prepare the infusion, solution dilution of enoblituzumab DP1 is performed in a syringe or an IV administration bag containing normal saline. The infusion solution is administered to the patient from the dose-prepared 0.9% sodium chloride IV bag, or syringe, with a commercially available IV pump and IV administration tubing set. As described in more detail below, stability and compatibility studies were performed with the dilution and storage of dose-prepared enoblituzumab up to 24 hours at 25° C. and IV administration of enoblituzumab using unfiltered and filtered IV infusion sets for 120-minute IV infusion periods.

In the initial compatibility studies, enoblituzumab DP composition was diluted in syringes of the same composition as those commonly used in the clinic, i.e., polypropylene, or in IV bags of the same composition as those commonly used in the clinic, i.e., polyolefin, which were held at 25° C. The dilution scheme in the test syringes followed a bracketing approach, whereby two doses (0.0450 mg/mL and 5.59 mg/mL) were tested in 20 mL polypropylene syringes with small bore IV extension sets, representing low and high dose concentrations. The dilution scheme in the test IV bags followed a bracketing approach, whereby multiple drug concentrations (0.0270 mg/mL, 0.135 mg/mL and 5.59 mg/mL) were tested in polyolefin IV bags (50 mL and 250 mL sizes) with standard IV administration sets, representing high and low dose enoblituzumab concentrations. A summary of the bracketing approach and materials used is shown in Table 17.

TABLE 17
Bracketing Approach for Dilution
of Enoblituzumab DP Composition

| Concentration | | | |
|---|---|---|---|
| Tested | | Administration Vessel | IV Administration Set |
| 0.045 | mg/mL | 20 mL polypropylene | Small bore IV extension set |
| | | syringe | |
| 5.59 | mg/mL | 20 mL polypropylene | Small bore IV extension set |
| | | syringe | |
| 0.027 | mg/mL | 250 mL polyolefin IV bag | Standard IV administration |
| | | | set |
| 0.135 | mg/mL | 50 mL polyolefin IV bag | Standard IV administration |
| | | | set |
| 5.59 | mg/mL | 50 mL polyolefin IV bag | Standard IV administration |
| | | 250 mL polyolefin IV bag | set |

Structural integrity of enoblituzumab was maintained under all conditions and time points as assessed by size exclusion chromatography (SE-HPLC) and protein concentration recovery by UV spectrophotometry. These studies support the stability of enoblituzumab and its compatibility for clinical administration when diluted in 0.9% sodium chloride in polypropylene syringes and polyolefin IV bags.

2.2. Evaluation of Enoblituzumab DP1 Composition In-Use Compatibility with Polypropylene Syringes and Small Bore IV Extension Sets

For the assessment of compatibility and stability of enoblituzumab in 20 mL polypropylene syringes, each of the two test concentrations of enoblituzumab was prepared in multiple syringes, and then were incubated at 25° C.

### 2.2.1. Study Design

Enoblituzumab was diluted with normal saline at concentrations 0.045 mg/mL and 5.59 mg/mL in either 20 mL polypropylene syringes containing normal saline (Table 15). Syringes were then held at 25° C. for 0, 4, 8 and 24 hours. Control samples with no exposure to polypropylene syringes were prepared in parallel and held at 2-8° C. for 0, 4, 8 and 24 hours. Samples were collected from each syringe upon completion of dose preparation (T=0) and at each of the time points (4, 8, and 24 hours). For each time point, samples were withdrawn from two syringes for analysis. Each syringe was only used for a single time point.

To assess compatibility and stability of enoblituzumab while passing through small bore IV extension sets (used with the 20 mL syringes), each of the two test concentrations of enoblituzumab was prepared in four 20 mL syringes, and incubated at 25° C. for 4-6 hours. The contents of two of the syringes at each concentration were then removed as Control samples for the IV extension set study. The remaining syringes were attached to small bore IV extension sets, and their contents passed through the extension sets over a period of 120 minutes using syringe pumps. The entire content of one syringe was collected into a single container and analyzed. Two administration sets were analyzed for each concentration.

### 2.2.2. Results

The results of these studies demonstrate that there were no significant changes observed in protein recovery (IgG concentration) and appearance and size distribution (SE-HPLC) for up to 24 hours incubation in polypropylene syringes or with passage through small bore IV extension sets for all testing groups. Recovery of enoblituzumab after incubating for up to 24 hours in the 20 mL syringes or after passage through the IV extension sets was ≥97.2%, with no significant change in the relative amount of IgG monomer (% Monomer), aggregates (% HMW), or the relative amount of fragments, clipped antibody forms, and free light or heavy chains (% LMW).

Representative results of the in-use compatibility studies with normal saline as administrative mixture with the use of polypropylene syringes and small bore IV extension sets are shown in Table 18 and Table 19. At each of the time points shown (0, 4, 8, and 24 hours), samples were withdrawn from two syringes for analysis; individual results for each of the two syringes are shown. Each syringe was used for only a single time point and analyzed for protein concentration (IgG % recovery) and structural integrity (by SE-HPLC).

TABLE 18
Compatibility and Stability of Enoblituzumab in 20 mL Polypropylene Syringes

| Test | | Time | IgG | SE-HPLC | | |
|---|---|---|---|---|---|---|
| Concentration | | Point | % Recovery ^{a} | % Monomer ^{b} | % HMW ^{c} | % LMW ^{d} |
| 0.0450 | mg/mL | Control | 104.0 | 99.1 | 0.9 | 0.1 |

| | | | | | |
|---|---|---|---|---|---|
| 0 | hr | 97.2 | 99.0 | 0.9 | 0.1 |
| | | 99.6 | 98.9 | 1.0 | 0.1 |
| 4 | hr | 103.0 | 98.9 | 0.9 | 0.2 |
| | | 97.2 | 98.7 | 1.2 | 0.1 |
| 8 | hr | 111.3 | 99.0 | 1.0 | 0.1 |
| | | 110.5 | 99.1 | 0.8 | 0.0 |
| 24 | hr | 111.1 | 99.0 | 1.0 | 0.0 |
| | | 113.5 | 99.2 | 0.7 | 0.0 |

| | | | | | | |
|---|---|---|---|---|---|---|
| 5.59 | mg/mL | Control | 98.9 | 98.6 | 1.4 | 0.1 |

| | | | | | |
|---|---|---|---|---|---|
| 0 | hr | 100.4 | 99.0 | 1.1 | 0.0 |
| | | 100.4 | 98.6 | 1.3 | 0.1 |
| 4 | hr | 101.6 | 98.7 | 1.3 | 0.0 |
| | | 101.8 | 98.5 | 1.5 | 0.0 |
| 8 | hr | 101.8 | 98.6 | 1.4 | 0.1 |
| | | 102.2 | 98.6 | 1.4 | 0.0 |
| 24 | hr | 102.4 | 98.9 | 1.1 | 0.1 |
| | | 102.0 | 98.3 | 1.7 | 0.1 |

TABLE 19
Compatibility and Stability of Enoblituzumab in Small Bore IV Extension Sets

| Test | | Time | IgG | SE-HPLC | | |
|---|---|---|---|---|---|---|
| Concentration | | Point | % Recovery ^{a} | % Monomer ^{b} | % HMW ^{c} | % LMW ^{d} |
| 0.0450 | mg/mL | Control | 106.2 | 98.7 | 1.2 | 0.1 |
| | | | 105.8 | 99.0 | 1.0 | 0.1 |
| | | Post-IV | 103.6 | 99.2 | 0.8 | 0.0 |
| | | tubing | 102.5 | 99.3 | 0.7 | 0.0 |
| 5.59 | mg/mL | Control | 100.4 | 99.0 | 0.8 | 0.0 |
| | | | 100.0 | 98.7 | 1.3 | 0.1 |
| | | Post-IV | 101.6 | 98.6 | 1.5 | 0.0 |
| | | tubing | 101.4 | 98.8 | 1.2 | 0.0 |

Footnotes used in Table 18 and Table 19:
a 3 IgG % Recovery is calculated as the measured IgG concentration of the test article, divided by the relevant reference value. The reference value for the Control sample was the theoretical test concentration. The reference value of the T=0, T=4, T=8, and T=24 hour samples, or the Post-IV Tubing samples, was the average measured value of the two Control samples.
b % Monomer is calculated as the area of the SE-HPLC monomer peak, divided by the sum of all peaks. % Monomer for each test sample should be compared to the Control samples of the same dose level.
c % HMW—High Molecular weight species , including dimers and larger forms, is calculated as the sum of all SE-HPLC peaks with apparent molecular weight greater than IgG monomer, divided by the sum of all peaks. % HMW for each sample is compared to the Control samples of the same dose level.
d LMW—Low Molecular Weight; species with apparent molecular weight less than IgG monomer, including antibody fragments and unassociated heavy or light chains. % LMW is calculated as the sum of all SE-HPLC peaks with apparent molecular weight less than IgG monomer, divided by the sum of all peaks. % LMW for each sample is compared to the Control samples of the same dose level.
   2.3. Evaluation of In-Use Compatibility of Enoblituzumab DP Composition with Polyolefin IV Bags and Standard IV Administration Sets

For the assessment of compatibility and stability of the enoblituzumab DP1 composition in polyolefin IV bags, 50 mL and 250 mL IV bags were prepared with their respective low and high concentrations of enoblituzumab DP1 composition (Table 16A) and then were incubated at 25° C.

### 2.3.1. Study Design

Enoblituzumab DP1 composition was diluted at three concentrations (0.027 mg/mL, 0.135 mg/mL, and 5.59 mg/mL) in polyolefin IV bags containing normal saline (50 mL or 250 mL). Two test bags were prepared for each size and concentration on separate days (Study 1 and Study 2). IV bags were then held at 25° C. for 0, 4, 8, and 24 hours. Control samples were prepared in parallel and held at 2-8° C. for 0, 4, 8, and 24 hours. For each time point, samples were withdrawn from each IV bag for analysis.

The low concentrations tested in IV bags were based on an estimated low dose of 6.75 mg, equivalent to a 45 kg clinical subject receiving the 0.15 mg/kg dose level. The respective low concentrations tested were 0.0270 mg/mL in the 250 mL IV bags and 0.135 mg/mL in the 50 mL IV bags. The high concentration tested was based on an estimated high dose of 1800 mg, equivalent to a 120 kg subject receiving the 15 mg/kg dose level. This dose was tested in 250 mL IV bags, giving a final concentration of 5.59 mg/mL (72.0 mL of 25 mg/mL drug product added to an IV bag containing 250 mL of saline). This concentration of 5.59 mg/mL was tested also as the upper bracketing concentration in the 50 mL IV bags.

Simulation of infusion through standard IV tubing sets was achieved by attaching tubing sets to IV bags after the completion of a 4-6 hour incubation of diluted enoblituzumab DP composition in the IV bag. Control sample (5 mL) was removed directly from the IV bag at the beginning of the infusion simulation, and the remaining infusion solution was allowed to pass through the tubing sets over approximately 120 minutes. The contents of one IV bag were collected into a single sample container, and analyzed. The three test concentrations were each tested twice, and with both unfiltered and filtered (sterile, non-pyrogenic, low-protein binding polyethersulfone (PES) 0.2 μM in-line filter) IV administration sets, on separate days (Study 1 and Study 2).

### 2.3.2. Results

The results of these studies demonstrate that there were no significant changes observed in protein recovery (IgG concentration) and appearance and size distribution (SE-HPLC) for up to 24 hours incubation in polyolefin IV bags in normal saline or with passage through standard IV extension sets for all testing groups. Recovery of enoblituzumab after incubating for up to 24 hours in the IV bags or after passage through the IV extension sets (unfiltered or filtered; over 120 minutes) was ≥85.9% and >95.2%, respectively, with no significant change in the relative amount of IgG monomer (% Monomer), aggregates (% HMW), or the relative amount of fragments, clipped antibody forms, and free light or heavy chains (% LMW).

Representative results of the in-use compatibility studies with normal saline as administrative mixture with the use of polyolefin IV bags and standard IV extension sets are shown in Tables 20-22. At each of the time points shown (0, 4, 8, and 24 hours), samples were withdrawn from the IV bag for analysis. Within each study (Study 1 and Study 2), a single bag was prepared at each test concentration, from which samples were withdrawn for T=0, 4, 8, and 24 hrs. Each sample was used for only a single time point and analyzed for protein concentration (IgG % recovery) and structural integrity (by SE-HPLC).

TABLE 20
Compatibility and Stability of Enoblituzumab in 50 mL Polyolefin IV Bags

| Test | Time | | IgG | SE-HPLC | | |
|---|---|---|---|---|---|---|
| Concentration | Point | | % Recovery ^{a} | % Monomer ^{b} | % HMW ^{c} | % LMW ^{d} |
| 0.135 mg/mL, | Control | | 103.7 | 99.1 | 0.9 | 0.0 |
| Study 1 | 0 | hr | 97.9 | 99.2 | 0.9 | 0.0 |
| | 4 | hr | 97.1 | 99.3 | 0.7 | 0.0 |
| | 8 | hr | 95.0 | 99.3 | 0.8 | 0.0 |
| | 24 | hr | 95.0 | 99.4 | 0.7 | 0.0 |
| 0.135 mg/mL, | Control | | 102.2 | 99.0 | 0.9 | 0.0 |
| Study 2 | 0 | hr | 97.1 | 99.0 | 0.9 | 0.0 |
| | 4 | hr | 96.4 | 99.3 | 0.6 | 0.0 |
| | 8 | hr | 95.7 | 99.3 | 0.7 | 0.0 |
| | 24 | hr | 94.2 | 99.2 | 0.8 | 0.0 |
| 5.59 mg/mL, | Control | | 101.1 | 99.0 | 1.0 | 0.0 |
| Study 1 | 0 | hr | 98.9 | 98.8 | 1.3 | 0.0 |
| | 4 | hr | 99.3 | 98.8 | 1.3 | 0.0 |
| | 8 | hr | 99.3 | 98.6 | 1.3 | 0.0 |
| | 24 | hr | 100.7 | 98.6 | 1.4 | 0.0 |
| 5.59 mg/mL, | Control | | 101.6 | 98.7 | 1.3 | 0.0 |
| Study 2 | 0 | hr | 98.6 | 98.7 | 1.3 | 0.0 |
| | 4 | hr | 98.4 | 98.6 | 1.4 | 0.0 |
| | 8 | hr | 97.9 | 98.9 | 1.2 | 0.0 |
| | 24 | hr | 98.4 | 98.7 | 1.3 | 0.0 |

TABLE 21
Compatibility and Stability of Enoblituzumab in 250 mL Polyolefin IV Bags

| Test | Time | | IgG | SE-HPLC | | |
|---|---|---|---|---|---|---|
| Concentration | Point | | % Recovery ^{a} | % Monomer ^{b} | % HMW ^{c} | % LMW ^{d} |
| 0.0270 mg/mL, | Control | | 92.2 | 99.4 | 0.6 | 0.0 |
| Study 1 | 0 | hr | 106.0 | 99.3 | 0.7 | 0.0 |
| | 4 | hr | 103.2 | 99.3 | 0.6 | 0.0 |
| | 8 | hr | 97.6 | 99.4 | 0.7 | 0.0 |
| | 24 | hr | 95.6 | 99.4 | 0.7 | 0.0 |
| 0.0270 mg/mL, | Control | | 99.6 | 98.9 | 1.1 | 0.0 |
| Study 2 | 0 | hr | 88.5 | 99.0 | 1.1 | 0.0 |
| | 4 | hr | 88.8 | 99.5 | 0.5 | 0.0 |
| | 8 | hr | 90.7 | 99.1 | 0.9 | 0.0 |
| | 24 | hr | 85.9 | 99.4 | 0.7 | 0.0 |
| 5.59 mg/mL, | Control | | 101.1 | 99.0 | 1.0 | 0.0 |
| Study 1 | 0 | hr | 98.9 | 98.8 | 1.3 | 0.0 |
| | 4 | hr | 99.8 | 98.8 | 1.3 | 0.0 |
| | 8 | hr | 99.1 | 98.7 | 1.3 | 0.0 |
| | 24 | hr | 101.2 | 98.8 | 1.2 | 0.0 |
| 5.59 mg/mL, | Control | | 101.6 | 98.7 | 1.3 | 0.0 |
| Study 2 | 0 | hr | 99.5 | 98.7 | 1.3 | 0.0 |
| | 4 | hr | 101.8 | 98.6 | 1.5 | 0.0 |
| | 8 | hr | 98.8 | 98.9 | 1.2 | 0.0 |
| | 24 | hr | 99.5 | 98.8 | 1.2 | 0.0 |

TABLE 22
Compatibility and Stability of Enoblituzumab in Standard IV Administration Sets

| Test | Time | IgG | SE-HPLC | | |
|---|---|---|---|---|---|
| Concentration | Point | % Recovery ^{a} | % Monomer ^{b} | % HMW ^{c} | % LMW ^{d} |

Unfiltered Standard IV Administration Sets

| | | | | | |
|---|---|---|---|---|---|
| 0.0270 mg/mL, | Control | 93.3 | 99.2 | 0.8 | 0.0 |
| Study 1 | Post-IV | 95.2 | 99.3 | 0.7 | 0.0 |
| | Tubing | | | | |
| 0.0270 mg/mL, | Control | 88.9 | 99.4 | 0.5 | 0.0 |
| Study 2 | Post-IV | 97.1 | 99.0 | 0.9 | 0.0 |
| | Tubing | | | | |
| 0.135 mg/mL, | Control | 100.7 | 99.2 | 0.8 | 0.0 |
| Study 1 | Post-IV | 100.0 | 99.2 | 0.8 | 0.0 |
| | Tubing | | | | |
| 0.135 mg/mL, | Control | 98.5 | 99.2 | 0.9 | 0.0 |
| Study 2 | Post-IV | 100.8 | 99.0 | 0.9 | 0.0 |
| | Tubing | | | | |
| 5.59 mg/mL, | Control | 101.4 | 98.7 | 1.2 | 0.0 |
| Study 1 | Post-IV | 99.3 | 98.5 | 1.5 | 0.1 |
| | Tubing | | | | |
| 5.59 mg/mL, | Control | 101.1 | 98.7 | 1.3 | 0.0 |
| Study 2 | Post-IV | 99.1 | 98.5 | 1.5 | 0.0 |
| | Tubing | | | | |

Filtered (0.2 mM) Standard IV Administration Sets

| | | | | | |
|---|---|---|---|---|---|
| 0.0270 mg/mL, | Control | 90.4 | 99.3 | 0.6 | 0.0 |
| Study 1 | Post-IV | 103.7 | 99.3 | 0.7 | 0.0 |
| | Tubing | | | | |
| 0.0270 mg/mL, | Control | 87.0 | 99.3 | 0.7 | 0.0 |
| Study 2 | Post-IV | 102.1 | 99.3 | 0.7 | 0.0 |
| | Tubing | | | | |
| 0.135 mg/mL, | Control | 100.7 | 99.2 | 0.8 | 0.0 |
| Study 1 | Post-IV | 98.5 | 99.2 | 0.8 | 0.0 |
| | Tubing | | | | |
| 0.135 mg/mL, | Control | 99.3 | 99.1 | 0.9 | 0.0 |
| Study 2 | Post-IV | 99.3 | 99.2 | 0.9 | 0.0 |
| | Tubing | | | | |
| 5.59 mg/mL, | Control | 101.1 | 98.8 | 1.1 | 0.0 |
| Study 1 | Post-IV | 99.5 | 98.7 | 1.2 | 0.0 |
| | Tubing | | | | |
| 5.59 mg/mL, | Control | 100.7 | 98.6 | 1.4 | 0.0 |
| Study 2 | Post-IV | 99.6 | 98.3 | 1.7 | 0.0 |
| | Tubing | | | | |

Footnotes used in Tables 20-22:
a IgG % Recovery is calculated as the measured IgG concentration of the test article, divided by the relevant reference value. The reference value for the Control sample was the theoretical test concentration. The reference value of the T=0, T=4, T=8, and T=24 hour samples, or the Post-IV tubing Samples, was the measured value of the Control sample from the same Study (1 or 2).
b % Monomer is calculated as the area of the SE-HPLC monomer peak, divided by the sum of all peaks. % Monomer for each test sample should be compared to the Control sample of the same dose level.
c % HMW—High Molecular weight species, including dimers and larger forms, is calculated as the sum of all SE-HPLC peaks with apparent molecular weight greater than IgG monomer, divided by the sum of all peaks. % Aggregate for each sample should be compared to the Control sample of the same dose level.
d LMW—Low Molecular Weight, species with apparent molecular weight less than IgG monomer, including antibody fragments and unassociated heavy or light chains. % LMW is calculated as the sum of all SE-HPLC peaks with apparent molecular weight less than IgG monomer, divided by the sum of all peaks. % LMW for each sample should be compared to the Control sample of the same dose level.

### 2.3.3. Conclusions of Enoblituzumab DP Composition Compatibility Studies

Enoblituzumab DP1 composition has been shown to be compatible with normal saline solutions in 20 mL polypropylene syringes at concentrations in the range of 0.05-5.6 mg/mL and in polyolefin IV bags at concentrations of 0.03-5.6 mg/mL. Enoblituzumab DP composition was also shown to be compatible with small bore IV extension sets and standard IV administration sets. The results support enoblituzumab IV solution administration time of 120 min and storage of IV bag preparation for up to 6 hours at room temperature and 24 hours at 2-8° C. Additionally, the results support the use of 0.2 μM in-line PES filters for intravenous infusion of enoblituzumab.

### Example 3

### Enoblituzumab DP1 Composition Long-Term and Accelerated Stability Studies

Long-term and accelerated stability studies of the enoblituzumab DP1 composition in stoppered 10 mL or 20 mL glass vials were performed. The stability was evaluated for an enoblituzumab DP1 composition stored in the recommended condition of 2-8° C. for up to 48 months and stored in the accelerated condition of 23-27° C. for up to 6 months.

### 3.1. Experimental Plan

A summary of the tests performed and the intervals generally evaluated in the 2-8° C. and 23-27° C. storage conditions are presented in Tables 23A and 23B, respectively. These studies were performed on 17 different lots of enoblituzumab DP1 composition. The majority of the studies were conducted with the vials upright and at least one was conducted with the vials inverted.

TABLE 23A
Stability Testing of Enoblituzumab DP1
Composition in Inverted Vials at 2-8° C.

| | Testing Interval (Months) | | | | | | | | | | |
|---|---|---|---|---|---|---|---|---|---|---|---|
| Test Description | 0 | 3 | 6 | 9 | 12 | 18 | 24 | 30^{c} | 33^{c} | 36 | 48 |
| Protein Concentra- | X | X | X | X | X | X | X | X | X | X | X |
| tion by A₂₈₀ | | | | | | | | | | | |
| Potency by B7-H3 | X | X | X | X | X | X | X | X | X | X | X |
| Binding | | | | | | | | | | | |
| Potency by FcγRIIIa | X | X | X | X | X | X | X | X | X | X | X |
| Binding | | | | | | | | | | | |
| SE-HPLC | X | X | X | X | X | X | X | X | X | X | X |
| Reduced CE-SDS | X | X | X | X | X | X | X | X | X | X | X |
| Non-Reduced SDS- | X | X | X | X | X | X | X | X | X | X | X |
| PAGE | | | | | | | | | | | |
| Sterility | X | NS | NS | NS | X | NS | X | NS | X | X | X |
| IE-HPLC | X | X | X | X | X | X | X | X | X | X | X |
| Appearance | X | X | X | X | X | X | X | X | X | X | X |
| pH | X | X | X | X | X | X | X | X | X | X | X |
| Osmolality | X | NS | X | NS | X | NS | X | X | X | X | X |
| Subvisible | X | NS | X | NS | X | NS | X | X | X | X | X |
| Particulates | | | | | | | | | | | |

TABLE 23B
Stability Testing of Enoblituzumab DP1 Composition
in Upright Vials at 23-27° C.

Testing Interval (Months)

| Test Description ^{a} | 0 | 1 | 3 | 5 | 6 |
|---|---|---|---|---|---|
| Protein Concentration by A₂₈₀ | X | X | X | X | X |
| Potency by B7-H3 Binding | X | X | X | X | X |
| Potency by FcγRIIIa Binding | X | X | X | X | X |
| SE-HPLC | X | X | X | X | X |
| Reduced CE-SDS | X | X | X | X | X |
| Non-Reduced SDS-PAGE | X | X | X | X | X |
| IE-HPLC | X | X | X | X | X |
| Appearance | X | X | X | X | X |
| pH | X | X | X | X | X |
| Osmolality | X | NS | NS | NS | X |
| Subvisible Particulates | X | X | X | X | X |

### 3.2. Results

The results of all the tests for a representative lot of the enoblituzumab DP composition stored for 48 months at 2-84C, and for 6 months at 25C, are presented in Tables 24A-24B, and Table 25, respectively. Additional details for assay potency, purity, and protein stability (monomers and acidic and basic variants) are provided in the summary below.

TABLE 24A
Stability Data, Enoblituzumab DP1 Composition
(2-8° C.; Inverted Vial)

| | | Time (Months) | | | | |
|---|---|---|---|---|---|---|
| Test | | 0 | 3 | 6 | 9 | 12 |
| Protein Concentration | | 24.9 | 25.0 | 25.0 | 25.1 | 25.2 |
| (mg/mL) | | | | | | |
| Potency, B7-H3 Binding (%) | | 96 | 88 | 93 | 83 | 104 |
| Potency, FcγRIIIa Binding | | 115 | 99 | 106 | 104 | 110 |
| (%) | | | | | | |
| SE-HPLC | % Mono | 97.7 | 97.6 | 97.5 | 97.3 | 97.3 |
| | % HMW | 2.1 | 2.3 | 2.4 | 2.6 | 2.6 |
| | % LMW | 0.2 | 0.2 | 0.1 | 0.1 | 0.1 |
| Reduced CE-SDS | | 97.8 | 98.6 | 98.0 | 97.9 | 98.5 |
| Non-Reduced SDS-PAGE | | 98.2 | 98.6 | 98.5 | 97.1 | 98.3 |
| IE-HPLC | % MCP | 21.6 | 26.1 | 25.8 | 29.9 | 28.0 |
| | % AV | 33.8 | 31.3 | 35.5 | 28.3 | 30.2 |
| | % BV | 44.6 | 42.6 | 38.7 | 41.8 | 41.8 |
| Appearance, Clarity | | C | C | C | C | C |
| Appearance, Color | | Y | PY | PY | PY | PY |
| Appearance, | | FNP, | FNP, | FNP, | FNP, | FNP, |
| Visible Particles | | CPP | FPP | FPP | FPP | FPP |
| pH | | 5.2 | 5.2 | 5.2 | 5.2 | 5.2 |
| Osmolality | | 300 | NS | 291 | NS | 305 |
| (mOsm/kg H₂O) | | | | | | |
| Subvisible | P ≥ 2 μm | 8182 | NS | 8160 | NS | 75708 |
| Particulates | P ≥ 10 μm | 130 | NS | 134 | NS | 252 |
| | P ≥ 25 μm | 3 | NS | 58 | NS | 2 |

| | | | | | |
|---|---|---|---|---|---|
| Sterility | NG | NS | NS | NS | NG |

TABLE 24B
Stability Data, Enoblituzumab DP1 Composition (2-8° C.; Inverted Vial)

| | | Time (Months) | | | | | |
|---|---|---|---|---|---|---|---|
| Test | | 18 | 24 | 30 | 33 | 36 | 48 |
| Protein Concentration | | 25.1 | 25.5 | 25.1 | 25.0 | 25.0 | 25.1 |
| (mg/mL) | | | | | | | |
| Potency, B7-H3 Binding | | 103 | 89 | 89 | 95 | 94 | 113 |
| (%) | | | | | | | |
| Potency, FcγRIIIa Binding | | 116 | 116 | 116 | 110 | 130 | 108 |
| (%) | | | | | | | |
| SE-HPLC | % Mono | 97.3 | 97.3 | 97.0 | 96.7 | 97.0 | 96.8 |
| | % HMW | 2.5 | 2.5 | 2.8 | 2.9 | 2.7 | 2.9 |
| | % LMW | 0.2 | 0.2 | 0.2 | 0.5 | 0.2 | 0.3 |
| Reduced CE-SDS | | 98.0 | 99.1 | 98.1 | 98.4 | 98.0 | 97.0 |
| Non-Reduced | | 98.0 | 98.5 | 97.2 | 95.0 | 97.3 | 95.9 |
| SDS-PAGE | | | | | | | |
| IE-HPLC | % MCP | 28.7 | 30.1 | 29.8 | 30.0 | 29.9 | 25.6 |
| | % AV | 33.1 | 28.8 | 28.9 | 28.2 | 28.2 | 33.5 |
| | % BV | 38.2 | 41.1 | 41.3 | 41.7 | 41.8 | 40.9 |
| Appearance, Clarity | | C | SO | C | C | SO | SO |
| Appearance, Color | | PY | PY | PY | PY | PY | L |
| Appearance, | | FNP, | FNP, | FNP, | FNP, | FNP, | FNP, |
| Visible Particles | | FPP | FPP | FPP | FPP | FPP | FPP |
| pH | | 5.2 | 5.2 | 5.2 | NA | 5.2 | 5.2 |
| Osmolality | | NS | 305 | 294 | 303 | 294 | 289 |
| (mOsm/kg H₂O) | | | | | | | |
| Subvisible | P ≥ 2 μm | NS | 80925 | 150765 | 349455 | 138794 | 241511 |
| Particulates | P ≥ 10 μm | NS | 1447 | 2378 | 4300 | 662 | 3458 |
| | P ≥ 25 μm | NS | 35 | 6 | 23 | 1 | 5 |

| | | | | | | |
|---|---|---|---|---|---|---|
| Sterility | NS | NG | NG | NG | NA | NG |

TABLE 25
Stability Data, Enoblituzumab DP1 Composition
(25 ± 2° C.; Upright Vial)

| | | Time (Months) | | | | |
|---|---|---|---|---|---|---|
| Test | | 0 | 1 | 3 | 5 | 6 |
| Protein Concentration (mg/mL) | | 24.9 | 25.0 | 25.0 | 26.0 | 25.0 |
| Potency, B7-H3 Binding (%) | | 96 | 93 | 79 | 75 | 68 |
| Potency, FcγRIIIa Binding (%) | | 115 | 105 | 93 | 93 | 106 |
| SE-HPLC | % Mono | 97.7 | 97.4 | 97.1 | 96.8 | 96.8 |
| | % HMW | 2.1 | 2.3 | 2.5 | 2.6 | 2.6 |
| | % LMW | 0.2 | 0.3 | 0.4 | 0.6 | 0.7 |
| Reduced CE-SDS | | 97.8 | 97.7 | 98.3 | 97.5 | 98.5 |
| Non-Reduced SDS-PAGE | | 98.2 | 98.6 | 97.8 | 98.7 | 98.6 |
| IE-HPLC | % MCP | 21.6 | 25.8 | 23.7 | 19.6 | 17.6 |
| | % AV | 33.8 | 34.0 | 33.2 | 36.2 | 37.1 |
| | % BV | 44.6 | 40.2 | 43.1 | 44.2 | 45.2 |
| Appearance, Clarity | | C | C | C | C | C |
| Appearance, Color | | Y | L | PY | PY | PY |
| Appearance, Visible Particles | | FNP, | FNP, | FNP, | FNP, | FNP, |
| | | CPP | CPP | CPP | CPP | FPP |
| pH | | 5.2 | 5.2 | 5.2 | 5.2 | 5.3 |
| Osmolality (mOsm/kg H₂O) | | 300 | NS | NS | NS | 297 |
| Subvisible | P ≥ 2 μm | 8182 | NS | NS | NS | 16020 |
| Particu- | P ≥ 10 μm | 130 | NS | NS | NS | 138 |
| lates | P ≥ 25 μm | 3 | NS | NS | NS | 5 |

The stability data for all lots of enoblituzumab DP1 composition investigated were within acceptable limits through 36 to 48 months at the intended long-term storage condition of 2-8° C., with the exception of subvisible particulates. Subvisible particulates (P≥2 m and P≥10 m) increased in some lots beginning around 12 months of storage at 2-8° C. No additional changes exceeding the variability of the analytical procedures were observed for other monitored parameters.

At the accelerated storage condition of 25±2° C., all lots investigated were within acceptable limits through 6 months. There were slight decreases were observed in potency (B7-H3 binding) and purity by SE-HPLC. These changes in potency and purity under accelerated conditions are not unexpected for proteins and the results were well within acceptable limits. Heterogeneity by IE-HPLC showed a moderate decrease in monomeric purity and moderate increases in acidic variants in some lots, including the representative lot. The acidic variants contain mainly deamidation products. No changes exceeding the variability of the analytical procedure were observed for any of the other monitored parameters under accelerated storage conditions, demonstrating a robust stability of the enoblituzumab DP1 composition.

### 3.3. Stability Conclusions

The above analyses of quantitative data from stability-indicating methods for multiple lots of the enoblituzumab DP1 composition supports a shelf-life of at least 24 months at the recommended storage condition of 2-8° C. The representative stability data shown in Tables 24A-24B and Table 25 indicates that all other tests, qualitative and semi- or non-quantitative, also remained within acceptable limits through at least 24 months and support a shelf-life at least about 24 months, with an upper limit of at least about 36 to at least about 48 months.

### Example 4

### Stability Studies for a Pharmaceutical Composition Containing High Concentrations of Enoblituzumab

Stable, antioxidant-free, pharmaceutical compositions comprising high concentrations of enoblituzumab in liquid compositions were prepared. Enoblituzumab was formulated at concentrations of 60 mg/mL (Ac60) or 120 mg/mL (Ac120) in 20 mM sodium acetate, 9% sucrose, and 0.01% PS80 at pH 4.8. Additionally, enoblituzumab was formulated at 120 mg/mL (His120) in 20 mM histidine hydrochloride (histidine-HCl), 9% sucrose 0.01% PS80 formulation at pH 5.4 to examine the impact of histidine on visual appearance and stability.

### 4.1. Evaluation of Visual Appearance

The impact of high concentrations of enoblituzumab, acetate and histidine-HCl buffers and pH (4.8 and 5.4) was evaluated by visual appearance of the mixtures at 2-8° C. and 25° C. Results of these studies are shown in Table 26 and Table 27. The product quality of enoblituzumab was evaluated by visual inspection and subvisible particulates. Clear appearance was achieved for 60 mg/mL enoblituzumab (Ac60) and 120 mg/mL enoblituzumab formulated in either acetate (Ac120) or histidine-HCl (His120) buffers after storage at 2-8° C. up to 3 months (Table 26). Histidine-HCl formulations became more opalescent than acetate formulations after 1 month of storage at 25° C. (Table 27). The visual observation in this study indicate that acetate buffer at lower pH is a better liquid formulation for enoblituzumab.

TABLE 26
Visual Appearance of Enoblituzumab High
Dose Formulations Stored at 2-8° C.

| | Enoblituzumab | | | |
|---|---|---|---|---|
| Formulation Code | Concentration | | T = 1 | T = 3 |
| and Composition | (mg/mL) | T = 0 | month | months |
| Ac60 | 60 | C, BY, | C, BY, | C, BY, |
| pH 4.8, 20 mM acetate, | | FPP | FPP | FPP |
| 9% sucrose, 0.01% PS80 | | | | |
| Ac120 | 120 | C, BY, | C, BY, | C, BY, |
| pH 4.8, 20 mM acetate, | | FPP | FPP | FPP |
| 9% sucrose, 0.01% PS80 | | | | |
| His120 | 120 | C, BY, | C, BY, | C, BY, |
| pH 5.4, 20 mM histidine- | | FPP | FPP | FPP |
| HCl, 9% sucrose, 0.01% | | | | |
| PS80 | | | | |

TABLE 27
Visual Appearance of Enoblituzumab High
Dose Formulations Stored at 25° C.

| | Enoblituzumab | | | |
|---|---|---|---|---|
| Formulation Code | Concentration | | T = 1 | T = 3 |
| and Composition | (mg/mL) | T = 0 | month | months |
| Ac60 | 60 | C, BY, | C, BY, | C, BY, |
| pH 4.8, 20 mM | | FPP | FPP | FPP |
| acetate, 9% sucrose, | | | | |
| 0.01% PS80 | | | | |
| Ac120 | 120 | C, BY, | C, BY, | C, BY, |
| pH 4.8, 20 mM | | FPP | FPP | MPP |
| acetate, 9% sucrose, | | | | |
| 0.01% PS80 | | | | |
| His120 | 120 | C, BY, | SO, BY, | SO, BY, |
| pH 5.4, 20 mM | | FPP | MPP | MPP |
| histidine-HCl, 9% | | | | |
| sucrose, 0.01% PS80 | | | | |

### 4.2. Short Term Stability Studies

Short term stability studies were conducted to monitor enoblituzumab stability in high concentration enoblituzumab formulations under normal storage conditions (2-8° C.), accelerated storage conditions (25° C.) and stressed (40±2° C.) conditions. The product quality of enoblituzumab was evaluated using high molecular weight species (0% HMW) (SE-HPLC) and charge variants distribution (SE-HPLC). The stability study results are summarized in Tables 28-33. The molecular weight analysis for samples stored at 2-8° C. (Table 28) showed that the % monomer decreased ˜0.6-0.9% over 3 months. A ˜2.4-3.0% decrease in the 0% monomer was observed in the accelerated 25° C. storage condition (Table 29). A larger reduction in % monomer was observed for the stressed 40° C. storage condition (Table 30) The differences in the percent of monomer for the samples stored at 2-8° C. were not significant among acetate formulations of 60 mg/mL and 120 mg/mL at pH4.8, as well as histidine formulation 120 mg/mL at pH5.4 shown by SEC.

TABLE 28
Short Term Stability SE-HPLC results at 2-8° C.

Formu-

| lation | T = 0 | | | 1 Month | | | 3 Months | | |
|---|---|---|---|---|---|---|---|---|---|
| Code | % H | % M | % L | % H | % M | % L | % H | % M | % L |
| Ac60 | 1.62 | 96.93 | 1.44 | 1.94 | 96.57 | 1.49 | 1.84 | 95.94 | 2.17 |
| Ac120 | 1.8 | 96.63 | 1.23 | 2.15 | 96.37 | 1.44 | 2.17 | 96.02 | 1.81 |
| His120 | 1.77 | 96.55 | 1.34 | 2.19 | 96.39 | 1.37 | 2.2 | 95.79 | 1.98 |

TABLE 29
Short Term Stability SE-HPLC results at 25° C.

Formu-

| lation | T = 0 | | | 1 Month | | | 3 Months | | |
|---|---|---|---|---|---|---|---|---|---|
| Code | % H | % M | % L | % H | % M | % L | % H | % M | % L |
| Ac60 | 1.62 | 96.93 | 1.44 | 1.94 | 95.93 | 2.13 | 1.85 | 94.74 | 3.41 |
| Ac120 | 1.8 | 96.63 | 1.23 | 2.44 | 95.41 | 2.11 | 2.63 | 93.69 | 3.69 |
| His120 | 1.77 | 96.55 | 1.63 | 2.40 | 95.46 | 2.13 | 2.67 | 93.71 | 3.62 |

TABLE 30
Short Term Stability SE-HPLC results at 40° C.

Formu-

| lation | T = 0 | | | 2 Weeks | | | 1 Month | | |
|---|---|---|---|---|---|---|---|---|---|
| Code | % H | % M | % L | % H | % M | % L | % H | % M | % L |
| Ac60 | 1.62 | 96.93 | 1.44 | 1.69 | 93.38 | 4.93 | 1.83 | 90.87 | 7.26 |
| Ac120 | 1.8 | 96.63 | 1.23 | 2.18 | 93.18 | 4.64 | 2.2 | 90.85 | 6.33 |
| His120 | 1.77 | 96.55 | 1.63 | 2.38 | 93.37 | 4.24 | 1.85 | 90.9 | 7.21 |

Abbreviations used in Tables 28-30: H=High Molecular Weight Species, M: monomer, L=Low Molecular Weight Species

The charge variant distribution for each formulation stored at 2-8C, 25° C., and 40° C. are shown in Tables 31, 32, and 33, respectively. Overall, the changes in the % main charge peak are not significant for the normal 2-8° C. storage condition. Under the accelerated 25° C. storage condition, at 120 mg/mL concentration, the % main charge peak decreased about 3.5%-4.5% Larger decreases in the % main charge peak were observed under the stressed 40° C. storage condition. No clear trends were observed for the 00 of acidic variants (AV) and 00 of basic variants (BV) at any of the conditions tested (2-8° C., 25° C. and 40° C.).

TABLE 31
Short Term Stability IE-HPLC results at 2-8° C.

| Formu- | T = 0 | | | 1 Month | | | 3 Months | | |
|---|---|---|---|---|---|---|---|---|---|
| lation | % | % | % | % | % | % | % | % | % |
| Code | AV | MCP | BV | AV | MCP | BV | AV | MCP | BV |
| Ac60 | 25.79 | 27.03 | 47.18 | 26.48 | 26.19 | 47.34 | 26.44 | 25.72 | 47.84 |
| Ac120 | 25.78 | 26.84 | 47.38 | 26.67 | 26.44 | 46.89 | 25.64 | 26.93 | 47.42 |
| His120 | 25.08 | 26.76 | 48.17 | 26.66 | 26.55 | 46.78 | 26.77 | 26.91 | 46.32 |

TABLE 32
Short Term Stability IE-HPLC results at 25° C.

| Formu- | T = 0 | | | 1 Month | | | 3 Months | | |
|---|---|---|---|---|---|---|---|---|---|
| lation | % | % | % | % | % | % | % | % | % |
| Code | AV | MCP | BV | AV | MCP | BV | AV | MCP | BV |
| Ac60 | 25.79 | 27.03 | 47.18 | 26.55 | 25.26 | 48.19 | 25.32 | 22.11 | 52.58 |
| Ac120 | 25.78 | 26.84 | 47.38 | 27.19 | 26.34 | 47.47 | 26.38 | 21.41 | 51.61 |
| His120 | 25.08 | 26.76 | 48.17 | 27.2 | 25.05 | 47.75 | 25.57 | 22.92 | 48.51 |

TABLE 33
Short Term Stability IE-HPLC results at 40° C.

| Formu- | T = 0 | | | 1 Month | | | 3 Months | | |
|---|---|---|---|---|---|---|---|---|---|
| lation | % | % | % | % | % | % | % | % | % |
| Code | AV | MCP | BV | AV | MCP | BV | AV | MCP | BV |
| Ac60 | 25.79 | 27.03 | 47.18 | 26.29 | 20.21 | 53.5 | 26.29 | 20.21 | 53.5 |
| Ac120 | 25.78 | 26.84 | 47.38 | 26.3 | 20.52 | 53.18 | 26.3 | 20.52 | 53.18 |
| His120 | 25.08 | 26.76 | 48.17 | 25.84 | 20.83 | 53.33 | 25.84 | 20.83 | 53.33 |

Abbreviations used in Tables 31-33: AV=acidic variants, MCP=main charge peak, BV=basic variants

In summary, the short-term stability studies demonstrated enoblituzumab is stable at high concentrations 60 mg/mL and 120 mg/mL when formulated in 20 mM acetate buffer with 9% sucrose, and 0.10% PS80 at pH 4.8 with no significant change in product quality for both molar weight distribution by SE-HPLC and charge variants distribution by IE-HPLC after 3 months of storage under normal storage conditions (2-8° C.) and accelerated storage conditions (25° C.).

### Example 5

### Materials and Methods

### 5.1. Protein Concentration by A₂₈₀

The protein concentration of enoblituzumab was determined by measuring absorbance of the sample in a cuvette using a UV spectrophotometer or by a SoloVPE system (SoloVPE Variable Pathlength UV System from C Technologies, Inc.).

For the cuvette method, the protein concentration was calculated using the following formula:

$$\\ Protein Concentration ( in mg / mL ) = [ ( corrected {A}_{280} ) / ε ] \times DF$$

where ε is and DF is the Dilution Factor of the sample preparation.

The SoloVPE system employs a Slope Spectroscopy method which is based on the Beer-Lambert Law and the slope derived from the linear regression of absorbance 280 nm measurements made at multiple path lengths. The protein concentration was calculated using the following Slope Spectroscopy equation:

$$\\ Protein Concentration ( mg / mL ) = c = M / ε$$

where c is the Concentration, M is the Slope of the regression line, and 8 is the Extinction Coefficient [1.43 (mg/mL)⁻¹cm⁻¹] that is calculated based on the enoblituzumab amino acid sequence.

### 5.2. Subvisible Particulates By HIAC Liquid Particle Counting

Subvisible particulate matter in the drug product was detected, sized, and counted utilizing the method described in USP<788> and Ph. Eur. 2.9.19. An electronic liquid-borne particle-counting system using a light obscuration sensor is employed. Particles were counted in three size ranges, ≥2 m (characterization information only), ≥10 m, and ≥25 m using an electronic liquid-borne particle-counting system using a light obscuration sensor (HIAC). Ten vials (10 mL/vial) of drug product are pooled for analysis.

### 5.3. Appearance

Appearance was assessed visually per USP<1>, Ph. Eur. 2.2.2 and Ph. Eur. 2.9.20 under visible light meeting minimum intensity requirements, in front of both a white and a black background. Sample aliquots were assessed in clear glass vials. Attributes examined include color of solution and clarity of solution. The degree of coloration was determined using Ph. Eur. certified color standards. The degree of clarity was determined using Ph. Eur. certified reference suspension standards.

### 5.4. pH Testing

The pH of a solution was measured potentiometrically using a calibrated pH meter, following compendial methods [USP<791>, Ph. Eur. 2.2.3]. Prior to testing samples the pH meter was 3 point calibrated using certified pH standards, starting with a pH 7 buffer standard, and then proceeding to a pH 4 and then pH 10 buffer standard. Following the calibration a system suitability check was performed using two certified pH buffers at pH 5 and pH 8.

### 5.5. Monomeric Purity By Size Exclusion High Performance Liquid Chromatography (SE-HPLC)

Size exclusion high performance liquid chromatography (SE-HPLC) was used as a measure of product purity and to measure impurities, particularly IgG aggregates. The assay includes enoblituzumab Reference Standard as a control sample for identity of the IgG monomer peak and for system suitability. Samples were injected onto a SE-HPLC column and are eluted isocratically with sodium phosphate/sodium sulfate buffer. Eluted proteins were detected using ultraviolet (UV) absorbance at 280 nm. The reportable result was the product purity, calculated as the area percent of the product monomer peak (compared to all peaks excluding the peaks of excipients). Also reported was the total percent of all species with apparent molecular weight greater than the IgG monomer (called High Molecular Weight species, or HMW), the percent of dimer (which is one potential component of the HMW species), and the total percent of all species with apparent molecular weight lower than the IgG monomer (called Low Molecular Weight species, or LMW).

### 5.6. Charge Heterogeneity and Identity by IE-HPLC

The charge heterogeneity and identity of enoblituzumab was evaluated by ion exchange high performance liquid chromatography (IE-HPLC). The assay includes an enoblituzumab Reference Standard as a control sample for identity and for system suitability. Samples were injected onto a Thermo WCX-10 column or equivalent, and were eluted with a salt gradient at constant pH. Eluted proteins are detected using ultraviolet (UV) absorbance at 280 nm. The reportable result was the main peak % area (percent of all peaks detected, excluding buffer/excipient peaks), and also the total % area of all acidic variants (AV; or APG, acidic peak group: peaks eluting prior to the main peak), and the total % area of all basic variants (BV; or BPG, basic peak group: peaks eluting after the main peak).

5.7. Charge Heterogeneity and Identity by cIEF

Alternatively, the charge heterogeneity and identity of enoblituzumab can be evaluated by capillary isoelectric focusing (cIEF). For example, cIEF can be performed using an iCE3 System with an Alcott 720NV Autosampler (ProteinSimple). For such analysis, an enoblituzumab Reference Standard and test article samples are prepared containing carrier ampholytes and pI markers, and are loaded into a capillary cartridge for analysis. The electrolytic tanks at each end of the capillary are filled with anolyte and catholyte solutions. Voltage is applied and the samples are focused at their pI. A camera takes a UV light absorption image of the entire capillary column at frequent regular intervals (e.g., every 30 seconds), allowing real time monitoring of the focusing step. The resulting separation pattern image is captured and analyzed with chromatography data system software. The test article electrophoretic profile is compared to the Reference Standard electrophoretic profile. The reportable results of the assay are the average main charge peak % area, the average acidic variants % area, and the average basic variants % area, of duplicate preparations.

To confirm identity, if required, the pI of the main peak of the test article should be within 0.5 pI units of the pI of the main peak of the enoblituzumab Reference Standard, and the test article profile must compare qualitatively to that of the Reference Standard, within a given sample set.

### 5.8. Purity by Reduced and Non-Reduced CE-SDS and LDS

Reduced and Non-reduced sodium dodecyl sulfate-capillary electrophoresis (CE-SDS) or sodium lauryl sulfate-capillary electrophoresis (CE-LDS) provides quantitative information on product purity, as well as qualitative information on the nature of impurities, adducts, product fragments, and covalently linked species. Samples for reduced method were denatured and reduced by heating in SDS sample buffer containing reducing agent 2-mercaptoethanol (OME). Samples were then electrophoresed using a Sciex PA800/PA800 Plus Capillary Electrophoresis System (formerly Beckman Coulter and AB Sciex). Test article and Reference Standard samples were loaded onto a capillary cartridge and product purity is determined by UV detection (220 nm). The reportable result for the test article was the % Purity, defined as the sum of the velocity-corrected peak area relative percentages corresponding to Heavy Chain and Light Chain, recorded to the nearest 0.1%. For non-reduced methods the samples were not reduced and analysis was performed similar to reduced samples. The % purity was defined as the area under the peak of the intact IgG molecule observed on the profile.

### 5.9. Purity by Non-Reduced SDS-PAGE

Non-reduced sodium dodecyl sulfate-polyacrylamide gel electrophoresis (SDS-PAGE) provides quantitative information on product purity, as well as qualitative information on the nature of impurities, adducts, product fragments, and covalently linked species. Samples were mixed with SDS-PAGE sample buffer without reducing agent. Samples were then electrophoresed using polyacrylamide gradient gels. Two concentrations of sample were loaded onto the gel for electrophoresis: a ‘low’ load used for quantitation, and a ‘high’ load used for qualitative comparison of the test article to the reference standard. Each test article was analyzed individually on a single gel and each gel also contained the enoblituzumab Reference Standard at low and high loads. Once electrophoresis was complete, gels were stained using a Coomassie blue stain, and then destained. Stained gels were scanned and analyzed using a digital imager and densitometry software. The area percent of each visible band was calculated, relative to the total area of all visible bands. The reportable result was the product purity, defined as the sum of the area percents (in the ‘low’ load lanes) of all bands representing intact, monomeric IgG. The area percent of a band was calculated relative to the total area of all visible bands.

### 5.10. Potency by B7-H3 Binding ELISA

An indirect enzyme-linked immunosorbent assay (ELISA) that quantitates binding activity of enoblituzumab to B7-H3 was used to assess the potency. Recombinant human B7-H3 was coated on the solid phase (96-well assay plates). enoblituzumab sample was allowed to bind to the immobilized B7-H3. A dilution series of the test article and of the enoblituzumab Reference Standard was tested in this manner, in order to generate dose-response curves. An alkaline phosphatase (AP)-conjugated anti-human kappa antibody was then added and allowed to bind to the complex of enoblituzumab and B7-H3. Quantitation of bound AP-conjugated antibody was achieved by addition of 4-Methylumbelliferyl Phosphate (4-MUP) substrate. Dephosphorylation of the added 4-MUP substrate by the AP-conjugated antibody yields a highly fluorescent stable product, 4-methylumbelliferon, which is measured using a fluorescence microplate reader. The level of fluorescence signal was proportional to the amount of captured enoblituzumab. Data were fitted to a four-parameter model to describe fluorescence signal as a function of enoblituzumab concentration. The reportable result, the potency of the test article relative to the enoblituzumab Reference Standard, was calculated using the following formula:

$$\\ Relative Potency = 100 \% \times {EC}_{50} B 7 ‐ H 3 Reference Standard / {EC}_{50} test article .$$

### 5.11. Potency and Identity by FcγRIIIa Binding ELISA

Potency of the Fc domain was assessed with an indirect competitive enzymelinked immunosorbent assay (ELISA) that quantitates binding activity of enoblituzumab to Fcγ Receptor subtype IIIa (FcγRIIIa), also referred to as CD16a. The quantification of the binding of enoblituzumab test article Fc to the FcγRIIIa was measured by its ability to compete against the binding of a biotin-labeled enoblituzumab competitor sample (enoblituzumab-Bt). To perform the assay, soluble recombinant human FcγRIIIa was coated on the solid phase (96-well assay plates). A dilution series of enoblituzumab test article in a constant concentration of enoblituzumab-Bt was allowed to bind to the immobilized FcγRIIIa. A dilution-series of the test article and the enoblituzumab Reference Standard was analyzed in this manner on the same assay plate, in order to generate dose-response curves for both the test article and the Reference Standard. Detection of bound enoblituzumab-Bt was achieved by addition of Alkaline Phosphatase conjugated with Streptavidin (Streptavidin-AP), followed by a colorimetric AP substrate. The intensity (absorbance) of the color signal was measured using a microplate reader. The level of color signal was proportional to the amount of bound enoblituzumab-Bt. Data were fitted to a four-parameter model to describe absorbance signal as a function of enoblituzumab concentration. The reportable result, the potency of the test article relative to the enoblituzumab Reference Standard, was calculated using the following formula:

$$\\ Relative Potency = 100 \% \times {EC}_{50} enoblituzumab Reference Standard / {EC}_{50} test article$$

### 5.12. Osmolality

Osmolality was measured with a freezing point depression osmometer using methods defined in the compendia [USP<785>, Ph. Eur. 2.2.35]. NIST-traceable standards were used for calibration at each measurement. System suitability was determined prior to measuring test articles by measuring a NIST-traceable standard.

### 5.13. Sterility

Sterility was tested using methods defined in USP <71> and Ph. Eur. 2.6.1.

All publications and patents mentioned in this specification are herein incorporated by reference to the same extent as if each individual publication or patent application was specifically and individually indicated to be incorporated by reference in its entirety.

The present disclosure is not to be limited in terms of the particular embodiments described in this application. Many modifications and variations may be made without departing from its spirit and scope, as will be apparent to those skilled in the art. Functionally equivalent methods and compositions within the scope of the disclosure, in addition to those enumerated herein, will be apparent to those skilled in the art from the foregoing descriptions. Such modifications and variations are intended to fall within the scope of the disclosure and/or the appended claims. It is to be understood that this disclosure is not limited to particular methods, compounds, or compositions, which may of course vary. It is also to be understood that the terminology used herein is for the purpose of describing particular embodiments only, and is not intended to be limiting.

What is claimed is:

1. A pharmaceutical composition comprising:
a) about 5 mg/mL to about 200 mg/mL enoblituzumab;
b) acetate;
c) sucrose;
d) polysorbate 80 (“PS80”); and
e) water.

2. The pharmaceutical composition of claim 1, wherein said acetate is present at a concentration of about 5 mM to about 30 mM.

3. The pharmaceutical composition of any one of claim 1 or 2, wherein said acetate comprises sodium acetate.

4. The pharmaceutical composition of any one of claims 1-3, wherein said composition comprises:
a) about 5 mM to about 30 mM acetate, about 50 mg/mL to about 130 mg/mL of sucrose, about 0.05 mg/mL to about 0.6 mg/mL of PS80, and water, wherein the composition has a pH of about 4.0 to about 6.0; or
b) about 8 mM to about 24 mM acetate, about 72 mg/mL to about 108 mg/mL of sucrose, about 0.05 mg/mL to about 0.6 mg/mL of PS80, and water, wherein the composition has a pH of about 4.4 to about 5.6; or
c) about 16 mM to about 24 mM acetate, about 72 mg/mL to about 108 mg/mL of sucrose, about 0.05 mg/mL to about 0.2 mg/mL of PS80, and water, wherein the composition has a pH of about 4.3 to about 5.3; or
d) about 8 mM to about 12 mM acetate, about 72 mg/mL to about 108 mg/mL of sucrose, about 0.05 mg/mL to about 0.2 mg/mL of PS80, and water, wherein the composition has a pH of about 4.5 to about 5.5; or
e) about 10 mM acetate, about 90 mg/mL of sucrose, about 0.1 mg/mL of PS80, and water, wherein the composition has a pH of about 4.6 to about 5.5; or
f) about 20 mM acetate, about 90 mg/mL of sucrose, about 0.1 mg/mL of PS80, and water, wherein the composition has a pH of about 4.4 to about 5.2.

5. The pharmaceutical composition of any one of claims 1-4, wherein said acetate comprises glacial acetic acid at a concentration of about 0.1 mg/mL to about 0.65 mg/mL and sodium acetate trihydrate at a concentration of about 0.6 mg/mL to about 1.8 mg/mL.

6. The pharmaceutical composition of any one of claims 1-5, wherein said enoblituzumab has a concentration of about 5 mg/mL to about 60 mg/mL.

7. The pharmaceutical composition of any one of claims 1-6, wherein said acetate comprises glacial acetic acid at a concentration of about 0.1 mg/mL to about 0.35 mg/mL and sodium acetate trihydrate at a concentration of about 0.60 mg/mL to about 1.2 mg/mL.

8. The pharmaceutical composition of any one of claims 1-7, wherein said acetate comprises glacial acetic acid at a concentration of about 0.18 mg/mL and sodium acetate trihydrate at a concentration of about 0.95 mg/mL.

9. The pharmaceutical composition of any one of claims 1-7, wherein said acetate comprises glacial acetic acid at a concentration of about 0.27 mg/mL and sodium acetate trihydrate at a concentration of about 0.74 mg/mL.

10. The pharmaceutical composition of any one of claims 1-5, wherein said enoblituzumab has a concentration of about 90 mg/mL to about 200 mg/mL.

11. The pharmaceutical composition of any one of claims 1-5 or 10, wherein said acetate comprises glacial acetic acid at a concentration of about 0.4 mg/mL to about 0.65 mg/mL and sodium acetate trihydrate at a concentration of about 1.2 mg/mL to about 1.8 mg/mL.

12. The pharmaceutical composition of any one of claims 1-5 or 10-11, wherein said acetate comprises glacial acetic acid at a concentration of about 0.52 mg/mL and sodium acetate trihydrate at a concentration of about 1.5 mg/mL.

13. The pharmaceutical composition of any one of claims 1-11, wherein said concentration of sucrose is about 90 mg/mL.

14. The pharmaceutical composition of any one of claims 1-12, wherein said concentration of PS80 is about 0.1 mg/mL.

15. The pharmaceutical composition of any one of claims 1-13, wherein said composition has a pH of about 4.6 to about 5.5.

16. The pharmaceutical composition of any one of claims 1-14, wherein said composition has a pH of about 4.4 to about 5.2.

17. The pharmaceutical composition of any one of claims 1-7 or 12-15, wherein said composition comprises about 25 mg/mL of enoblituzumab, about 0.18 mg/mL of glacial acetic acid, about 0.95 mg/mL of sodium acetate trihydrate, about 90 mg/mL of sucrose, about 0.1 mg/mL of PS80, and water, wherein the composition has a pH of about 4.7 to about 5.5.

18. The pharmaceutical composition of any one of claims 1-7 or 12-15, wherein said composition comprises about 25 mg/mL of enoblituzumab, about 0.27 mg/mL of glacial acetic acid, about 0.74 mg/mL of sodium acetate trihydrate, about 90 mg/mL of sucrose, about 0.1 mg/mL of PS80, and water, wherein the composition has a pH of about 4.6 to about 5.4.

19. The pharmaceutical composition of any one of claims 1-4, 9-14 or 16, wherein said composition comprises about 120 mg/mL of enoblituzumab, about 0.52 mg/mL of glacial acetic acid, about 1.5 mg/mL of sodium acetate trihydrate, about 90 mg/mL of sucrose, about 0.1 mg/mL of PS80, and water, wherein the composition has a pH of about 4.4 to about 5.2.

20. The pharmaceutical composition of any one of claims 1-19, wherein said composition does not comprise an antioxidant.

21. The pharmaceutical composition of any one of claims 1-19, wherein said composition has a shelf-life of at least about 18 months at about 2° C. to about 8° C.

22. The pharmaceutical composition of any one of claims 1-19, wherein said composition has a shelf-life of at least about 24 months at about 2° C. to about 8° C.

23. The pharmaceutical composition of any one of claims 1-19, wherein said composition has a shelf-life of at least about 36 months at about 2° C. to about 8° C.

24. The pharmaceutical composition of any one of claims 1-19, wherein said composition has a shelf-life of at least about 48 months at about 2° C. to about 8° C.

25. The pharmaceutical composition of any one of claims 1-24, wherein said composition has an osmolality of about 200 to about 400 mOsm/kg H₂O.

26. The pharmaceutical composition of any one of claims 1-24, wherein said composition has an osmolality of about 260 to about 360 mOsm/kg H₂O.

27. The pharmaceutical composition of any one of claims 1-26, wherein said composition maintains monomeric purity of said enoblituzumab for at least about 3 months at about 25° C.

28. The pharmaceutical composition of any one of claims 1-26, wherein said composition maintains monomeric purity of said enoblituzumab for at least about 18 months at about 2° C. to about 8° C.

29. The pharmaceutical composition of any one of claims 1-28, wherein said composition maintains the heterogeneity profile of said enoblituzumab for about for at least about 3 months at 25° C.

30. The pharmaceutical composition of any one of claims 1-29, wherein said composition maintains the heterogeneity profile of said enoblituzumab for about for at least about 18 months at about 2° C. to about 8° C.

31. The pharmaceutical composition of any of claims 1-30, wherein said water is sterile, nonpyrogenic, distilled water.

32. The pharmaceutical composition of any one of claims 1-31, wherein said composition is sterile.

33. A container comprising the pharmaceutical composition of any one of claims 1-32.

34. The container of claim 33, wherein said container comprises about 10 mL volume of said pharmaceutical composition, wherein volume comprises:
a) about 250 mg enoblituzumab;
b) about 10 mM sodium acetate
c) about 900 mg sucrose;
d) about 1 mg PS80; and
e) water; and
wherein said composition has a pH of about 4.6 to about 5.5.

35. The container of claim 33, wherein said container comprises about 10 mL volume of said pharmaceutical composition, wherein said volume comprises:
a) about 250 mg enoblituzumab;
b) about 1.8 mg glacial acetic acid
c) about 9.5 mg sodium acetate trihydrate;
d) about 900 mg sucrose;
e) about 1 mg PS80; and
f) water; and
wherein said composition has a pH of about 4.7 to about 5.5.

36. The container of claim 33, wherein said container comprises about 10 mL volume of said pharmaceutical composition, wherein said volume comprises:
a) about 250 mg enoblituzumab;
b) about 2.7 mg glacial acetic acid
c) about 7.4 mg sodium acetate trihydrate;
d) about 900 mg sucrose;
e) about 1 mg PS80; and
f) water; and
wherein said composition has a pH of about 4.6 to about 5.4.

37. The container of claim 33, wherein said container comprises about 10 mL volume of said pharmaceutical composition, wherein said volume comprises:
a) about 1,200 mg enoblituzumab;
b) about 20 mM sodium acetate;
c) about 900 mg sucrose;
d) about 1 mg PS80; and
e) water; and
wherein said composition has a pH of about 4.4 to about 5.2.

38. The container of claim 33, wherein said container comprises about 10 mL volume of said pharmaceutical composition, wherein said volume comprises:
a) about 1,200 mg enoblituzumab;
b) about 5.2 mg glacial acetic acid
c) about 15 mg sodium acetate trihydrate;
d) about 900 mg sucrose;
e) about 1 mg PS80; and
f) water; and
wherein said composition has a pH of about 4.4 to about 5.2.

39. The container of claim 33, wherein said container comprises about 17 mL volume of said pharmaceutical composition, wherein said volume comprises:
a) about 425 mg enoblituzumab;
b) about 10 mM;
c) about 1530 mg sucrose;
d) about 1.7 mg PS80; and
e) water; and
wherein said composition has a pH of about 4.6 to about 5.4.

40. The container of claim 33, wherein said container comprises about 17 mL volume of said pharmaceutical composition, wherein said volume comprises:
a) about 425 mg enoblituzumab;
b) about 3.06 mg glacial acetic acid
c) about 16.15 mg sodium acetate trihydrate;
d) about 1530 mg sucrose;
e) about 1.7 mg PS80; and
f) water; and
wherein said composition has a pH of about 4.7 to about 5.5.

41. The container of claim 33, wherein said container comprises about 17 mL volume of said pharmaceutical composition, wherein said volume comprises:
a) about 425 mg enoblituzumab;
b) about 4.59 mg glacial acetic acid
c) about 12.58 mg sodium acetate trihydrate;
d) about 1530 mg sucrose;
e) about 1.7 mg PS80; and
f) water; and
wherein said composition has a pH of about 4.6 to about 5.4.

42. A sealed package comprising the pharmaceutical composition of any one of claims 1-32 or the container of any one of claims 33-41.

43. A kit comprising the pharmaceutical composition of any one of claims 1-32, the container of any one of claims 33-41, or the sealed package of claim 42 and optionally further comprising instructions for administration of the pharmaceutical composition to a subject in need thereof.

44. A sealed package comprising the pharmaceutical composition of any one of claims 1-32, or the container of any one of claims 33-41, or the kit of claim 43, and optionally further comprising instructions for administration of the pharmaceutical composition to a subject in need thereof.

45. A method of treating cancer, comprising administering enoblituzumab to a subject in need thereof using the pharmaceutical composition according to any one of claims 1-32, the container of any one of claims 33-41, the sealed package of any one of claim 42 or 44, or the kit of any one of claim 43 or 44.

46. The method of claim 45, wherein said method comprises:
a) diluting the pharmaceutical composition in a container comprising 0.9% sodium chloride or D5W, to obtain a dosing solution;
b) inverting the container to mix the diluted solution; and
c) attaching the container containing the dosing solution to a device for administration to said subject.

47. The method of claim 46, wherein the container is an IV bag or a syringe containing 0.9% sodium chloride.

48. The method of claim 46, wherein the container is an IV bag or a syringe containing D5W.

49. Use of the pharmaceutical composition of any one of claims 1-32, for the production of a medicament for the treatment of cancer in a subject in need thereof.

50. Use of the pharmaceutical composition according to any one of claims 1-32, the container of any one of claims 33-41, the sealed package of any one of claim 42 or 44, or the kit of any one of claim 43 or 44, for the treatment of cancer in a subject in need thereof.

51. The use of any one of claim 49 or 50, wherein said use comprises:
a) diluting the pharmaceutical composition in a container comprising 0.9% sodium chloride or D5W, to obtain a dosing solution;
b) inverting the container to mix the diluted solution; and
c) attaching the container containing the dosing solution to a device for administration to said subject.

52. The use of claim 51, wherein the container is an IV bag or syringe containing 0.9% sodium chloride.

53. The use of claim 51, wherein the container is an IV bag or syringe containing D5W.

54. The method of any one of claims 45-48, or the use of any one of claims 49-53, wherein said dosing solution maintains monomeric purity of said enoblituzumab for about 6 hours at about 25° C. or for about 24 hours at about 2° C. to about 8° C.

55. The method of any one of claims 45-48 or 54, or the use of any one of claims 49-54, wherein said administration is by IV infusion for at least about 30 minutes.

56. The method of any one of claims 45-48 or 54, or the use of any one of claims 49-54, wherein said administration is by IV infusion for at least about 60 minutes.

57. The method of any one of claims 45-48 or 54, or the use of any one of claims 49-54, wherein said administration is by IV infusion for at least about 120 minutes.

58. The method of any one of claims 45-48 or 54-57, or the use of any one of claims 49-57, wherein said pharmaceutical composition is diluted to obtain a weight-based treatment dose of about 6 mg/kg to about 15 mg/kg.

59. The method of any one of claims 45-48 or 54-58, or the use of any one of claims 49-58, wherein said pharmaceutical composition is diluted to obtain a weight-based treatment dose of about 15 mg/kg

60. The method of any one of claims 45-48, or 54-59, or the use of any one of claims 49-59, wherein said administration of the dosing solution is once every 3 weeks.

61. The method of any one of claims 45-48 or 54-60, or the use of any one of claims 49-60, wherein said cancer expresses B7-H3.

62. The method of any one of claims 45-48 or 54-61, or the use of any one of claims 49-61, wherein said cancer is selected from the group consisting of: adrenal gland cancer, AIDS-associated cancer, alveolar soft part sarcoma, anal cancer, squamous cell carcinoma of the anal canal (SCAC), bladder cancer, bone cancer, brain and spinal cord cancer, breast cancer, HER2⁺ breast cancer, Triple-Negative Breast Cancer (TNBC), carotid body tumor, cervical cancer, HPV-related cervical cancer, chondrosarcoma, chordoma, chromophobe renal cell carcinoma, clear cell carcinoma, colon cancer, colorectal cancer, desmoplastic small round cell tumor, ependymoma, endometrial cancer, unselected endometrial cancer, MSI-high endometrial cancer, dMMR endometrial cancer, POLE exonuclease domain mutation positive endometrial cancer, Ewing's sarcoma, extraskeletal myxoid chondrosarcoma, gallbladder cancer, bile duct cancer, cholangiocarcinoma bile duct cancer, gastric cancer, gastroesophageal junction (GEJ) cancer, gestational trophoblastic disease, germ cell tumor, glioblastoma, head and neck cancer, squamous cell carcinoma of head and neck (SCCHN), a hematological malignancy, a hepatocellular carcinoma, islet cell tumor, Kaposi's Sarcoma, kidney cancer, leukemia, acute myeloid leukemia, liposarcoma/malignant lipomatous tumor, liver cancer, hepatocellular carcinoma liver cancer (HCC), lymphoma, diffuse large B-cell lymphoma (DLBCL), non-Hodgkin's lymphoma (NHL), lung cancer, small cell lung cancer (SCLC), non-small cell lung cancer (NSCLC), medulloblastoma, melanoma, uveal melanoma, meningioma, Merkel cell carcinoma, mesothelioma, mesothelial pharyngeal cancer, multiple endocrine neoplasia, multiple myeloma, myelodysplastic syndrome, neuroblastoma, neuroendocrine tumors, ovarian cancer, pancreatic cancer, papillary thyroid carcinoma, parathyroid tumor, pediatric cancer, peripheral nerve sheath tumor, pharyngeal cancer, pheochromocytoma, pituitary tumor, prostate cancer, metastatic castration resistant prostate cancer (mCRPC), posterious uveal melanoma, renal metastatic cancer, rhabdoid tumor, rhabdomyosarcoma, sarcoma, skin cancer, a small round blue cell tumor of childhood, neuroblastoma, rhabdomyosarcoma, soft-tissue sarcoma, squamous cell cancer, stomach cancer, synovial sarcoma, testicular cancer, thymic carcinoma, thymoma, thyroid cancer, urothelial cancer, and uterine cancer.

63. The method of any one of claim 61 or 62, or the use of any one of claim 61 or 62, wherein said cancer is anal cancer, bladder cancer, breast cancer, bile duct cancer, cervical cancer, colorectal cancer, endometrial cancer, gastric cancer, GEJ cancer, head and neck cancer, liver cancer, lung cancer, lymphoma, ovarian cancer, prostate cancer, skin cancer, and urothelial cancer.

64. The method of any one of claim 62 or 63, or the use of any one of claim 62 or 63, wherein said anal cancer is SCAC.

65. The method of any one of claim 62 or 63, or the use of any one of claim 62 or 63, wherein said lung cancer is NSCLC.

66. The method of any one of claim 62 or 63, or the use of any one of claim 62 or 63, wherein said breast cancer is TNBC.

67. The method of any one of claim 62 or 63, or the use of any one of claim 62 or 63, wherein said skin cancer is melanoma or Merkel cell carcinoma.

68. The method of any one of claim 62 or 63, or the use of any one of claim 62 or 63, wherein said head and neck cancer is SCCHN.

69. The method of any one of claim 62 or 63, or the use of any one of claim 62 or 63, wherein said prostate cancer is mCRPC.

70. The method of any one of claim 62 or 63, or the use of any one of claim 62 or 63, wherein said cancer is urothelial cancer.

71. The method of any one of claims 45-48 or 54-70, or the use of any one of 49-70, wherein said subject is a human subject.
